(19) United States
(12) Patent Application Publication
(10) Pub. No.: US 2025/0313649 A1
(43) Pub. Date: Oct. 9, 2025

(54) COMPOSITIONS AND METHODS FOR TARGETED PROTEIN DEGRADATION

(71) Applicant: THE CHILDREN'S MEDICAL CENTER CORPORATION, Boston, MA (US)

(72) Inventor: Stuart H. ORKIN, Brookline, MA (US)

(21) Appl. No.: 18/863,427

(22) PCT Filed: May 5, 2023
(86) PCT No.: PCT/US2023/066674

§ 371 Date: Nov. 6, 2024

Related U.S. Application Data
(60) Provisional application No. 63/339,208, filed on May 6, 2022.

(51) Int. Cl. C07K 16/40; C12N 15/62; C12N 15/864

(52) U.S. Cl. CPC C07K 16/40; C12N 15/62; C12N 15/8645; C07K 2317/569; C07K 2317/622; C07K 2319/01

(57) ABSTRACT

Described herein are compositions and methods for targeted erythroid-specific protein degradation. The compositions described comprise a binding domain for a erythroid protein of interest, e.g., BCL11A, and a binding domain for a erythroid enriched E3 ubiquitin ligase. Compositions and methods described can be used to promote fetal hemoglobin and treat hemoglobinopathy disorders.

### CROSS-REFERENCE TO RELATED APPLICATIONS

This application includes a claim of priority under 35 U.S.C. 119 (e) to U.S. provisional patent application No. 63/339,208, filed May 6, 2022, the entirety of which is hereby incorporated by reference.

### GOVERNMENT SUPPORT

This invention was made with government support under Grant Number HL032259, awarded by the National Institutes of Health. The Government has certain rights in the invention.

### SEQUENCE LISTING

The instant application contains a Sequence Listing that has been submitted in XML format via Patent Center and is hereby incorporated by reference in its entirety. Said XML copy, created on May 4, 2023, is named “701039-191700WOPT_SL2.xml” and is 93,396 bytes in size.

### BACKGROUND

Targeted protein degradation (TPD) in cells is most commonly achieved with molecular glues or proteolysis-targeting chimeras (PROTACs). In the majority of instances, PROTACs leverage widely expressed E3 ubiquitin ligases (or their associated subunits), e.g. cereblon or von Hippel-Lindau (VHL) proteins, to recruit a protein of interest (POI) to the proteasome for degradation. In order to restrict TPD to specific cells, rather than risk TPD in many different cell types, it would be advantageous to leverage cell-restricted E3 ubiquitin ligases.

### SUMMARY

The technology described herein relates, in part, to the discovery that certain ubiquitin ligases are enriched for expression in cells of the erythroid lineage. Where ubiquitination marks proteins for ubiquitin-mediated degradation, this discovery provides approaches for the targeted degradation of a desired target polypeptide in an erythroid-preferring manner. In one approach, a bispecific construct including binding domains for a target polypeptide and an erythroid-enriched E3 ubiquitin ligase can act as a tether, bringing the target polypeptide into close proximity with the erythroid-enriched E3 ubiquitin ligase. Where the E3 ubiquitin ligase involved is expressed in an erythroid-enriched manner, such a construct will function to selectively channel the target polypeptide into the degradation pathway in erythroid cells. In another approach, the E3 ubiquitin ligase itself is fused to a molecule that specifically binds a desired target polypeptide. Similar to the first approach, tethering of the E3 ubiquitin ligase to a degradation target in this manner results in the ubiquitination and subsequent degradation of the tethered target. Compositions for targeting desired polypeptides for degradation and methods of using them are described herein. In various embodiments, the constructs described herein can be used therapeutically to target desired polypeptides expressed in erythroid cells for degradation. Non-limiting examples include the transcription factor BCL11A, which is involved in repression of fetal hemoglobin (HbF) expression in adult erythroid lineage cells. Targeting compositions and methods described herein can promote the degradation of BCL11A in erythroid cells, thereby promoting re-expression of HbF in those cells, which can provide therapeutic benefit for subjects suffering from β-hemolglobinopathies.

In one aspect, described herein is a fusion polypeptide comprising a binding domain that specifically binds an erythroid-enriched E3 ubiquitin ligase and a binding domain that specifically binds an erythroid-enriched polypeptide of interest.

In one embodiment of this and any other aspect described herein, the fusion polypeptide mediates degradation of the polypeptide of interest in erythroid cells.

In another embodiment of this and any other aspect described herein, the binding domain that specifically binds an erythroid-enriched E3 ubiquitin ligase specifically binds TRIM10 or TRIM58.

In another embodiment of this and any other aspect described herein, the binding domain that specifically binds an erythroid-enriched E3 ubiquitin ligase comprises an antibody or antigen-binding fragment thereof. In another embodiment of this and any other aspect described herein, the antibody or antigen-binding fragment thereof comprises an scFv, a single domain antibody or a nanobody.

In another embodiment of this and any other aspect described herein, the binding domain that specifically binds an erythroid-enriched polypeptide of interest comprises an antibody or antigen-binding fragment thereof. In another embodiment of this and any other aspect described herein, the antibody or antigen-binding fragment thereof comprises an scFv, a single domain antibody or a nanobody.

In another embodiment of this and any other aspect described herein, the polypeptide of interest is selected from BCL11A, LRF and ZNF410.

In another embodiment of this and any other aspect described herein, the E3 ubiquitin ligase is TRIM10 or TRIM58.

In another embodiment of this and any other aspect described herein, the polypeptide of interest is BCL11A, and the binding domain that specifically binds the polypeptide of interest binds BCL11A at an epitope comprised by zinc-finger 6 (ZNF6) of the BCL11A polypeptide.

In another embodiment of this and any other aspect described herein, the polypeptide of interest is BCL11A, and the binding domain that specifically binds the polypeptide of interest binds BCL11A at an epitope comprised by zinc finger 23 (ZNF23) of the BCL11A polypeptide. With regard to nomenclature, as used herein, “ZNF 23” is shorthand for zinc fingers 2 and 3, or a polypeptide fragment of BCL11A including those zinc fingers, and “ZNF456” is shorthand for zinc fingers 4, 5 and 6, or a polypeptide fragment of BCL11A including those zinc fingers.

In another embodiment of this and any other aspect described herein, the polypeptide of interest is BCL11A, and the binding domain that specifically binds the polypeptide of interest has an amino acid sequence at least 90% identical to SEQ ID NO: 12. In another embodiment of this and any other aspect described herein, amino acid sequence variation relative to SEQ ID NO: 12 occurs at one or more of amino acids according to Table 2. In some embodiments, variation relative to SEQ ID NO: 12 occurs only at only one site, two sites, three sites, four sites, five sites, six sites, seven sites, eight sites, 9 sites, 10 sites, 11 sites of 12 sites as set out in Table 2.

In another embodiment of this and any other aspect described herein, the polypeptide of interest is BCL11A, and the binding domain that specifically binds BCL11A has an amino acid sequence at least 90% identical to SEQ ID NO: 34. In another embodiment of this and any other aspect described herein, amino acid sequence variation relative to SEQ ID NO: 34 occurs at one or more of amino acids at amino acid number 102 or 108. In another embodiment of this and any other aspect described herein, variation occurs only at amino acids 102 or 108 relative to SEQ ID NO: 34.

In another embodiment of this and any other aspect described herein, the polypeptide of interest is BCL11A, and the binding domain that specifically binds BCL11A comprises a single-domain antibody in which: CDR1 has an amino acid sequence selected from SEQ ID NOs: 43-50; CDR2 has an amino acid sequence selected from SEQ ID NOs: 51-59; and CDR3 has an amino acid sequence selected from SEQ ID NOs 60-65.

In another embodiment of this and any other aspect described herein, the polypeptide of interest is BCL11A, and the binding domain that specifically binds BCL11A comprises a single-domain antibody in which: CDR1 has the amino acid sequence SIFVNNAM (SEQ ID NO: 37); CDR2 has the amino acid sequence ELVAAISASGGSTYY (SEQ ID NO: 38); and CDR3 has a sequence selected from ADQDVYPYEYW (SEQ ID NO: 39), ADQDGYPYEYW (SEQ ID NO: 40) and ADQDVYPYEYL (SEQ ID NO: 41).

In another embodiment of this and any other aspect described herein, the fusion polypeptide further comprises a cell-penetrating peptide.

In another embodiment of this and any other aspect described herein, the fusion polypeptide further comprises a linker peptide between the binding domain that specifically binds an erythroid-enriched E3 ubiquitin ligase and the binding domain that specifically binds an erythroid-enriched polypeptide of interest.

In another aspect, described herein is a nucleic acid comprising sequence encoding a fusion polypeptide as described herein.

In one embodiment of this and any other aspect described herein, the sequence encoding the fusion polypeptide is operatively linked to regulatory sequences that permit expression in erythroid cells.

In another aspect, described herein is a vector comprising a nucleic acid comprising sequence encoding a fusion polypeptide as described herein. In one embodiment of this and any other aspect described herein, the vector is a viral vector. In another embodiment of this and any other aspect described herein, the viral vector is an AAV vector.

In another aspect, described herein is a method of erythroid-specific, targeted degradation of a protein of interest, the method comprising introducing a fusion polypeptide as described herein, a nucleic acid comprising sequence encoding a fusion polypeptide as described herein or a vector comprising sequence encoding a fusion polypeptide as described herein to an erythroid cell.

In another aspect, described herein is a method of targeted degradation of BCL11A, the method comprising introducing a fusion polypeptide as described herein, a nucleic acid comprising sequence encoding a fusion polypeptide as described herein or a vector comprising sequence encoding a fusion polypeptide as described herein to a cell expressing BCL11A.

In one embodiment of this and any other aspect described herein, the targeted degradation of BCL11A is targeted to erythroid cells.

In another aspect, described herein is a method of promoting fetal hemoglobin (HbF) expression in adult erythroid cells, the method comprising introducing a fusion polypeptide as described herein, a nucleic acid comprising sequence encoding a fusion polypeptide as described herein or a vector comprising sequence encoding a fusion polypeptide as described herein to a cell, wherein the protein of interest is BCL11A, and wherein the fusion polypeptide promotes the degradation of BCL11A in erythroid cells, thereby promoting HbF expression.

In another aspect, described herein is a method of treating a hemoglobinopathy disorder, the method comprising introducing a fusion polypeptide as described herein, a nucleic acid comprising sequence encoding a fusion polypeptide as described herein or a vector comprising sequence encoding a fusion polypeptide as described herein to a subject in need thereof, wherein the protein of interest is BCL11A, and wherein the fusion polypeptide promotes the degradation of BCL11A in erythroid cells, thereby promoting HbF expression to treat the hemoglobinopathy disorder.

In another aspect, described herein is a fusion polypeptide comprising a TRIM10 or TRIM58 polypeptide fused to a binding domain that specifically binds a target polypeptide.

In one embodiment of this and any other aspect described herein, the binding domain that specifically binds a target polypeptide comprises an antigen-binding domain of an antibody.

In another embodiment of this and any other aspect described herein, the binding domain that specifically binds a target polypeptide comprises an scFv, a single domain antibody or a nanobody.

In another embodiment of this and any other aspect described herein, the target polypeptide is expressed in an erythroid cell.

In another embodiment of this and any other aspect described herein, the target polypeptide is selected from BCL11A, LRF and ZNF410.

In another embodiment of this and any other aspect described herein, the polypeptide of interest is BCL11A, and the binding domain that specifically binds the polypeptide of interest binds BCL11A at an epitope comprised by zinc-finger 6 (ZNF6) of the BCL11A polypeptide.

In another embodiment of this and any other aspect described herein, the polypeptide of interest is BCL11A, and the binding domain that specifically binds the polypeptide of interest binds BCL11A at an epitope comprised by zinc finger 23 (ZNF23) of the BCL11A polypeptide.

In another embodiment of this and any other aspect described herein, the polypeptide of interest is BCL11A, and the binding domain that specifically binds the polypeptide of interest has an amino acid sequence at least 90% identical to SEQ ID NO: 12. In another embodiment of this and any other aspect described herein, amino acid sequence variation relative to SEQ ID NO: 12 occurs at one or more of amino acids according to Table 2. In some embodiments, variation relative to SEQ ID NO: 12 occurs only at only one site, two sites, three sites, four sites, five sites, six sites, seven sites, eight sites, 9 sites, 10 sites, 11 sites of 12 sites as set out in Table 2.

In another embodiment of this and any other aspect described herein, the polypeptide of interest is BCL11A, and the binding domain that specifically binds BCL11A has an amino acid sequence at least 90% identical to SEQ ID NO: 34. In another embodiment of this and any other aspect described herein, amino acid sequence variation relative to SEQ ID NO: 34 occurs at one or more of amino acids number 102 or 108. In another embodiment of this and any other aspect described herein, amino acid sequence variation relative to SEQ ID NO: 34 occurs only at amino acids number 102 or 108.

In another embodiment of this and any other aspect described herein, the polypeptide of interest is BCL11A, and the binding domain that specifically binds BCL11A comprises a single-domain antibody in which: CDR1 has an amino acid sequence selected from SEQ ID NOs: 43-50; CDR2 has an amino acid sequence selected from SEQ ID NOs: 51-59; and CDR3 has an amino acid sequence selected from SEQ ID NOs 60-65.

In another embodiment of this and any other aspect described herein, the polypeptide of interest is BCL11A, and the binding domain that specifically binds BCL11A comprises a single-domain antibody in which: CDR1 has the amino acid sequence SIFVNNAM (SEQ ID NO: 37);

CDR2 has the amino acid sequence ELVAAISASGGSTYY (SEQ ID NO: 38); and CDR3 has a sequence selected from ADQDVYPYEYW (SEQ ID NO: 39), ADQDGYPYEYW (SEQ ID NO: 40) and ADQDVYPYEYL (SEQ ID NO: 41).

In another embodiment of this and any other aspect described herein, the fusion polypeptide further comprises a cell-penetrating peptide.

In another embodiment of this and any other aspect described herein, the fusion polypeptide further comprises a linker peptide between the TRIM10 or TRIM58 polypeptide and the binding domain that specifically binds a target polypeptide.

In another aspect, described herein is a nucleic acid comprising sequence encoding a fusion polypeptide as described herein.

In one embodiment of this and any other aspect described herein, the sequence encoding the fusion polypeptide is operatively linked to regulatory sequences that permit expression in erythroid cells.

In another aspect, described herein is a vector comprising a nucleic acid comprising sequence encoding a fusion polypeptide as described herein. In one embodiment of this and any other aspect described herein, the vector is a viral vector. In another embodiment of this and any other aspect described herein, the viral vector is an AAV vector.

In another aspect, described herein is a method of erythroid-specific, targeted degradation of a protein of interest, the method comprising introducing a fusion polypeptide as described herein, a nucleic acid encoding a fusion polypeptide as described herein, or a vector comprising a nucleic acid encoding a fusion polypeptide as described herein to an erythroid cell.

In another aspect, described herein is a method of targeted degradation of BCL11A, the method comprising introducing a fusion polypeptide as described herein, a nucleic acid encoding a fusion polypeptide as described herein, or a vector comprising a nucleic acid encoding a fusion polypeptide as described herein to a cell expressing BCL11A.

In one embodiment of this and any other aspect described herein, the targeted degradation of BCL11A is targeted to erythroid cells.

In another aspect, described herein is a method of promoting fetal hemoglobin (HbF) expression in adult erythroid cells, the method comprising introducing a fusion polypeptide as described herein, a nucleic acid encoding a fusion polypeptide as described herein, or a vector comprising a nucleic acid encoding a fusion polypeptide as described herein to a cell, wherein the protein of interest is BCL11A, and wherein the fusion polypeptide promotes the degradation of BCL11A in erythroid cells, thereby promoting HbF expression.

In another aspect, described herein is a method of treating a hemoglobinopathy disorder, the method comprising introducing a fusion polypeptide as described herein, a nucleic acid encoding a fusion polypeptide as described herein, or a vector comprising a nucleic acid encoding a fusion polypeptide as described herein to a subject in need thereof, wherein the protein of interest is BCL11A, and wherein the fusion polypeptide promotes the degradation of BCL11A in erythroid cells, thereby promoting HbF expression to treat the hemoglobinopathy disorder.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-O-911910⁻¹⁹-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “increased”, “increase”, “enhance”, “activate” are all used herein to refer to an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10⁻¹⁰⁰% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “improve” or “improvement,” when applied to a score in a standardized scale or rating, e.g., for disease symptoms or severity, means a statistically significant, favorable change in the scale or rating on that scale.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to a reference level, for example, a decrease of at least about 20%, or at least about 30%, or at least about 40%, or at least about 50%, or at least about 60%, or at least about 70%, or at least about 80%, or at least about 90%, or at least about 95% as compared to a reference level. As used herein, “reduction” or “inhibition” does not encompass a complete inhibition or reduction as compared to a reference level. “Complete inhibition” is a 100% inhibition as compared to a reference level.

As used herein, a “reference level” refers to the level or value for a given parameter against which one compares the level or value in a given sample or situation to determine whether the level or value has changed in a meaningful way. A reference level can be a level in or from a sample that is not treated to change the parameter. A reference level can alternatively be a level in or from a normal or otherwise unaffected sample. A reference level can alternatively be a level in or from a sample obtained from a subject at a prior time point, for example, prior to a given treatment.

As used herein, an “appropriate control” refers to an untreated, otherwise identical cell or population (e.g., a subject who was not administered an agent described herein, or was administered by only a subset of agents described herein, as compared to a non-control cell).

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE FIGURES

FIGS. 1A-1C show erythroid cell enriched expression of TRIM10 and TRIM48 (FIG. 1A) The modular structures of TRIM10 and TRIM58. (FIG. 1B) RNA expression of E3 ligases during cell maturation. (FIG. 1C) Heat-map depicting the relative expression of UBE20, TRIM10, and TRIM58 across cell types. The dots indicate erythroid cells.

FIG. 2 shows fusion of TRIM10/58 to the recognition domain of cereblon (CRBN). Schematic showing the fusion proteins of TRIM10/58 with the CRBN recognition domain along with control constructs.

FIG. 3 shows degradation of BCL11A protein by TRIM10/58. Western blot for BCL11A depicting fusions of TRIM10/58 with the recognition domain of CRBN were capable of degrading tagged BCL11A upon addition of dTAG47 PROTAC. WT-CRBN served as a positive control and inactive form of dTAG47 served as a negative control.

FIG. 4 shows degradation of BCL11A protein by TRIM10/58 in HUDEP-2 erythroid cells. HUDEP-2 cells with CRBN or lacking CRBN were infected with lentiviruses expressing CRBN cDNA or TRIM10 fused with the recognition domain of CRBN and then dTAG47 PROTAC was added. BCL11A protein was degraded in the presence of dTAG47 in CRBN-cells rescued with CRBN cDNA and the fusion of TRIM10 with the recognition domain of CRBN leads to a reduction in BCL11A protein.

FIG. 5 shows targeted protein degradation of BCL11A by TRIM10/58 in 293T cells. Western blot for BCL11A of 293T cells transfected with constructs expressing either TRIM10 or TRIM58 fused to a BCL11A-specific nanobody (Nb19). The nanobody provides the recognition domain to direct the TRIM to BCL11A.

### DETAILED DESCRIPTION

The technology described herein relates, in part, to the discovery that certain ubiquitin ligases are enriched for expression in cells of the erythroid lineage. In particular, it was discovered that the E3 ubiquitin ligases TRIM10 and TRIM58 are expressed in an erythroid-enriched or erythroid-specific manner. Where E3 ubiquitin ligases target proteins in the cells in which they are expressed for degradation via the ubiquitin-mediated proteasome pathway, the identification of erythroid-specific or erythroid enriched E3 ubiquitin ligases provides avenues for erythroid specific, targeted degradation of a protein of interest. The ability to target a protein of interest for degradation in an erythroid-enriched manner has therapeutic implications, in that, as but one example, hemoglobinopathies generally stem from defects in cells of the erythroid lineage, and targeted degradation of, e.g., BCL11A, which represses expression of fetal hemoglobin (HbF), can de-repress and promote the expression of HbF, providing a new source of active hemoglobin.

Described herein below are constructs for targeting proteins of interest for degradation in cells of the erythroid lineage, as well as formulations and uses of such constructs for the treatment of diseases or disorders including, but not limited to hemoglobinopathies. The following describes the various considerations for one of skill in the art to practice the described technology.

### Ubiquitin-Mediated Protein Degradation

The degradation of proteins occurs mainly through two major pathways: autophagy and the ubiquitin proteasome system (UPS), both of which are critical for maintaining cellular homeostasis (see, e.g., Dikic, I., Ann. Rev. Biochem. 86:193-224 (2017)). The UPS is a cascade reaction, and an important mechanism for the degradation of misfolded and damaged proteins, as well as a regulated mechanism that ensures short lives of certain regulatory proteins, among others. Degradation of a protein via the ubiquitin pathway generally involves two discrete and successive steps: (a) covalent attachment of multiple ubiquitin molecules to the protein substrate-so called “ubiquitination” or “ubiquitylation;” and (b) degradation of the ubiquitin-targeted protein by the 26S proteasome complex, with the release of free and reusable ubiquitin. As used herein, “ubiquitination” or “ubiquitylation” refers to the post-translational modification of a protein by the covalent attachment (via an isopeptide bond) of one or more ubiquitin monomers.

The ubiquitin-proteasome system consists of several components that act in concert. One of these, ubiquitin, an evolutionarily conserved protein of 76 residues (human ubiquitin has the amino acid sequence: MQIFVKTLTGKTITLEVEPSDTIENVKAKIQDKEGIPPDQQRLIFAGKQLEDGRTLSDY NIQKESTLHLVLRLRGG (SEQ ID NO: 1), is activated in its C-terminal Gly to a high-energy thiol-ester intermediate. This reaction is catalyzed by the ubiquitin-activating enzyme, E1. After ubiquitin activation, one of several ubiquitin-carrier proteins or ubiquitin-conjugating enzymes, referred to as E2s, transfers the activated ubiquitin moiety from E1 to a member of the ubiquitin-protein ligase family, E3, to which the substrate protein is bound. E3 catalyzes the last step in the conjugation process, covalent attachment of ubiquitin to the substrate protein. The first ubiquitin moiety is transferred to the ε—NH₂ group of a lysine residue of the protein substrate to generate an isopeptide bond. In successive reactions, a polyubiquitin chain is generated by processive transfer of additional activated ubiquitin moieties to a lysine residue (one of the most prominently used is Lys48, or K48, but each of K6, K11, K27, K29, K33, K63 and the N-terminal methionine 1 (Met1) are also functional sites in various ubiquitination reactions) of the previously conjugated ubiquitin molecule. The polyubiquitin chain serves as a recognition marker for the 26S proteasome. The binding of the substrate to E3 is generally specific and indicates that E3 ubiquitin ligases play a major role in recognition and selection of proteins for conjugation and subsequent degradation. The structure of the system appears to be hierarchical: a single E1 appears to carry out activation of ubiquitin required for all modifications. Several major species of E2 enzymes have been characterized in mammalian cells, each of which can act with one or more E3 enzymes.

### E3 Ubiquitin Ligases

Hundreds of different E3 ubiquitin ligases have been identified. These are reviewed and classified in, e.g., Yang et al., Molecular Biomedicine 2:23 (2021). Different types of E3 ubiquitin ligases have low sequence homology and large differences in composition. The tripartite motif (TRIM) protein family is a subset of the so-called RING-type E3 ligase subfamily. The TRIM family of E3 ubiquitin ligases is reviewed, e.g., by Esposito et al., Biochem. Soc. Trans. 45:183-191 (2017). As discussed in the Examples herein, it was discovered that two E3 ubiquitin ligases, TRIM10 and TRIM58, are expressed in an erythroid-enriched manner. As used herein, the term “erythroid enriched” refers to the preferential expression and activity of the TRIM10 and TRIM58 E3 ubiquitin ligases in cells of the erythroid lineage. By preferential in this context is meant that TRIM10 and TRIM58 are most highly expressed in cells of this lineage, relative to their expression in other cell types or tissues. The erythroid-enriched expression of TRIM10 and TRIM58 renders these E3 ubiquitin ligase polypeptides useful for the targeted degradation of a protein of interest in an erythroid-enriched manner.

In some embodiments of any of the aspects, the E3 ubiquitin ligase is TRIM10. TRIM10 is an E3 ubiquitin ligase that plays an essential role in the differentiation and survival of terminal erythroid cells. Sequences of TRIM10 are known for a number of species, e.g., human TRIM10 (NCBI GeneID: 10107), mRNA (e.g., Isoform 1: NCBI Ref Seq NM_006778.4, SEQ ID NO: 2) and polypeptide (e.g., Isoform 1: NCBI Ref Seq: NP_006769.2, SEQ ID NO: 3). TRIM10 can refer to human TRIM10, including naturally occurring variants, molecules, and alleles thereof. For example, SEQ ID NO: 2 is a mRNA sequence for TRIM10. Note: U's replaced with T's in this representation.

| | |
|---|---|
| 1 | cttctgacag tggacagctg acatatcacc actttccttc tactgtgagt gtctctggat |
| 61 | gggcagaaag gaatggccag cccctggtta tggtcatcta aggtcacctc tgaaatgctg |
| 121 | tgagcccctc ttccttcctc tcctctgcta tttcccatct ctgctgttgg caggagaata |
| 181 | gaaccctggc tgccagagat gcaagtgtgt gacgatatgg gtgctggtgc atatttagta |
| 241 | tgtgcctgtg tccagccatg tgcatgtgtg ggtgtgtgag tgtgtgaccc agcccttccc |
| 301 | ccgtggccaa gcagagagag tggccttgag gaagccatag cagcaggacc agcatggcct |
| 361 | ctgctgcctc tgtgaccagc ctggcagatg aagtcaactg ccccatctgt cagggtaccc |
| 421 | tgagggagcc ggtcactatc gactgcggcc acaacttctg ccgggcctgc cttacccgct |
| 481 | actgtgagat accaggccca gacctggagg agtcccctac ttgcccactc tgcaaagaac |
| 541 | ccttccgtcc tgggagcttc cggcccaact ggcagctggc taacgtggtg gagaacattg |
| 601 | agcgcctcca gctggtgtcc acactgggtt tgggagagga ggatgtctgc caagagcacg |
| 661 | gagagaagat ctacttcttc tgtgaggatg atgagatgca gttgtgcgtg gtgtgccggg |
| 721 | aggctgggga gcacgctacc cacaccatgc gcttcctgga ggatgcagcg gctccctata |
| 781 | gggaacaaat ccataagtgt cttaaatgtc taagaaaaga gagagaggag attcaagaaa |
| 841 | tccagtcaag agaaaataaa aggatgcaag tcctcctgac tcaggtgtcc accaagagac |
| 901 | aacaggtgat ttctgagttc gcacacctga ggaagtttct agaggaacag cagagcatcc |
| 961 | tcttagcaca attggagagc caggatgggg acatcttgag gcaacgggat gaatttgatt |
| 1021 | tgctggttgc tggggagatc tgccggttta gtgctcttat tgaagaactg gaggagaaga |
| 1081 | atgagaggcc agcaagggag ctcctgacgg acatcagaag cactctaata agatgtgaaa |
| 1141 | ccagaaagtg ccggaaaccg gtggctgtgt cgccagagct gggccagagg attcgggact |
| 1201 | ttccccagca ggccctcccg ctgcagaggg agatgaagat gtttctggaa aaactatgct |
| 1261 | ttgagttgga ctatgagcca gctcacattt ctctagaccc tcagacttcc caccccaagc |
| 1321 | tcctcttgtc cgaggaccac cagcgagctc agttctccta caaatggcag aactcaccag |
| 1381 | acaaccccca acgttttgac cgggccacct gtgttctggc ccacactggc atcacagggg |
| 1441 | ggagacacac gtgggtggtg agtatagacc tggcccatgg gggcagctgc accgtgggcg |
| 1501 | tggtgagcga ggatgtgcag cggaaggggg agcttcggct gcggccagag gagggggtgt |
| 1561 | gggctgtgag gctggcttgg ggcttcgtct cggctctggg ctccttcccc acacggctga |
| 1621 | ccctgaagga gcagccccgg caggtgaggg tgtctcttga ctatgaggtg ggctgggtga |
| 1681 | ccttcaccaa cgctgtcacc cgagagccca tctacacctt cactgcctcc ttcactagga |
| 1741 | aggtcattcc cttctttggg ctctggggcc gagggtccag tttctccctg agctcctgag |
| 1801 | aaggagcagt tacctactct cctctaagta caggactcat atcaacccaa gtaccatgtg |
| 1861 | gacttgatcc ctggctgaat cacctggatg acttggaata gaaatgactg ctttagaaga |
| 1921 | tgggatgggg ccgggtggta agggatagaa gagaggactc tcaatctact gatcaagtcc |
| 1981 | tttccccaat gcccagtgga tggccagggt acctggggac tcaggctgct gccagttctg |
| 2041 | ctcaccacca tccgtgcttg gcacagaagt agctgcatag aaagggcact ggatttgaag |
| 2101 | tcagaagacc tgggttcttg aaccagcctg tcaaccagtt gtatgacttt aaacaaggca |
| 2161 | tctcacctct tttcatcttg ttttcttcca ataatgttag agttcatgta atcacattct |
| 2221 | ctagaaccat ttagtttgtg ttaactatga accaagcagt gtggtggcca ctggtgactt |
| 2281 | gaaaatatag agaaaaaaaa aacctgctct atatctgaaa gagctcttgg gaagacagag |
| 2341 | aaacataaag aggaaattac agcacagtgt ggtgggtgtt acgggaagtc caaccccagc |
| 2401 | attatgggag ttcaggggaa ggggcatagc ccagcctgga aggagagggt gagtggggga |
| 2461 | tggctttctg aaaagggtgg tctaaaggat gcctatggtc aatagggaaa agaggggaag |
| 2521 | aagcatctta agaagaggaa acagcagaga actggctgga tgacctgtga ctctggagca |
| 2581 | ctgggttgtc tccattgtca ttatgggcag atgtgtgcca tccccagccg acgccactca |
| 2641 | ctgcctcctt cctcctggtg ttgccacttc tgggtgagat taaggtgcag ggcctggggg |
| 2701 | caggaggaca taaggtatag ccataaatca taacccaggg accacactca accctaggga |
| 2761 | aattgtcttc ctgatcagtt gattaccatc tgaggtcaag aaatgagata gtgggagcaa |
| 2821 | atgggtcaca aatagctcag ctgtgggctc agaaactgct aggtaaaaga attccagaag |
| 2881 | gaggccaggg cataagttgg atgacctatg aactttagtc taaagaattg agactaccgt |
| 2941 | aattgagact actgtagtga catctgagaa atgggatgga agagtgacca tgttttattt |
| 3001 | tcttgttctt gtcactattg tattttattt tgcataatca tgcccttcac tgacagtctc |
| 3061 | cttaacatca tctgtttact ctgctcagtg taaactacaa tgctctgtca tctccctact |
| 3121 | gggtctcctg ggaggagggg agccatccag ggtgcaaact caaaggcaga gggcacagcg |
| 3181 | tgcttaggcc caagcttaga attcaattga gaagttctgt tcttcatcct tacctcagca |
| 3241 | ggtagaaaag aggtggagat cagaagccag ggattagaga ttgaattgct ttccctggga |
| 3301 | gtgtgcagta tctcattaaa atgttgtata ttcaaaaaaa tacagacaca cacaagtgcc |
| 3361 | tatataatga taaaacatat ccaaagcacc caaacctgtg attccagaaa agtagatcct |
| 3421 | atttttgttt atttttattg gctttagctg tgtcttttga aaggactatt atcctctaaa |
| 3481 | atgtatgtgt atgaagatcc tggcttgcag ctcgggagct ttacacacct tgtgtcactt |
| 3541 | tatcttggta acgagccaag atcatgcaac cagttcatcc atgtctgacc caagagctct |
| 3601 | taactgctat gctgcactgc ctcattcaga gatagatgcc tgcatgggtc ctggcgatta |
| 3661 | ttttaatgct ggctacaccc ccacaggtga cttggattca gaaatacaat ttatatttct |
| 3721 | tctttttaaa ttgttttatt ttatttttct tttaatagtt atatgtcagt gagaacaatt |
| 3781 | tatatgtctt acactgagaa ataaaactgc tcataagtga aaaca |

SEQ ID NO: 3 is an amino acid sequence of TRIM10.

```
    1 masaasvtsl adevncpicq gtlrepvtid cghnfcracl tryceipgpd leesptcplc
  61 kepfrpgsfr pnwqlanvve nierlqlvst lglgeedvcq ehgekiyffc eddemqlcvv
121 creagehath tmrfledaaa pyreqihkcl kclrkereei qeiqsrenkr mqvlltqvst
181 krqqvisefa hlrkfleeqq sillaqlesq dgdilrqrde fdllvageic rfsalieele
241 eknerparel ltdirstlir cetrkcrkpv avspelgqri rdfpqqalpl qremkmflek
301 lcfeldyepa hisldpqtsh pklllsedhq raqfsykwqn spdnpqrfdr atcvlahtgi
361 tggrhtwvvs idlahggsct vgvvsedvqr kgelrlrpee gvwavrlawg fvsalgsfpt
421 rltlkeqprq vrvsldyevg wvtftnavtr epiytftasf trkvipffgl wgrgssfsls
481 s
```

In some embodiments of any of the aspects, the E3 ubiquitin ligase is TRIM58. TRIM58 is an E3 ubiquitin ligase that plays a role during late erythropoiesis and erythroblast enucleation. Sequences of TRIM58 are known for a number of species, e.g., human TRIM58 (NCBI GeneID: 25893), mRNA (e.g., NCBI Ref Seq NM_015431.4, SEQ ID NO: 4) and polypeptide (e.g., NCBI Ref Seq: NP_056246.3, SEQ ID NO: 5). TRIM58 can refer to human TRIM58, including naturally occurring variants and alleles thereof. Antibodies that specifically bind TRIM58 are known; see, e.g., Novusbio Trim58 Antibody (2H3) H00025893-M02. One can generate monoclonal antibodies using standard procedures e.g., Corti et al, Microbiol Spectr 2 (2014), Huang et al. Nat Protoc 10, 1907-1915 (2013), and Pedrioli et al., Trends Immunol. 42:1143-1158 (2021), which are incorporated herein by reference. Single domain antibody fragments or nanobodies can be screened using a fully in vitro platform based on yeast surface display as in McMahon et al Nature 25:289-296 (2018).

SEQ ID NO: 4 is a mRNA sequence for TRIM58. Note: U's replaced with T's in this representation.

| | |
|---|---|
| 1 | gcagaccgcg aggggagacg gtgcgggcgg ccgggagcgc agccctccgg gaggcgggtc |
| 61 | atggcctggg cgccgcccgg ggagcggctg cgcgaggatg cgcggtgccc ggtgtgcctg |
| 121 | gatttcctgc aggagccggt cagcgtggac tgcggccaca gcttctgcct caggtgcatc |
| 181 | tccgagttct gcgagaagtc ggacggcgcg cagggcggcg tctacgcctg tccgcagtgc |
| 241 | cggggcccct tccggccctc gggctttcgc cccaaccggc agctggcggg cctggtggag |
| 301 | agcgtgcggc ggctggggtt gggcgcgggg cccggggcgc ggcgatgcgc gcggcacggc |
| 361 | gaggacctga gccgcttctg cgaggaggac gaggcggcgc tgtgctgggt gtgcgacgcc |
| 421 | ggccccgagc acaggacgca ccgcacggcg ccgctgcagg aggccgccgg cagctaccag |
| 481 | gtaaagctcc agatggctct ggaacttatg aggaaagagt tggaggacgc cttgactcag |
| 541 | gaggccaacg tggggaaaaa gactgtcatt tggaaggaga aagtggaaat gcagaggcag |
| 601 | cgcttcagat tggagtttga gaagcatcgt ggctttctgg cccaggagga gcaacggcag |
| 661 | ctgaggcggc tggaggcgga ggagcgagcg acgctgcaga gactgcggga gagcaagagc |
| 721 | cggctggtcc agcagagcaa ggccctgaag gagctggcgg atgagctgca ggagaggtgc |
| 781 | cagcgcccgg ccctgggtct gctggagggt gtgagaggag tcctgagcag aagtaaggct |
| 841 | gtcacaaggc tggaagcaga gaacatcccc atggaactga agacagcatg ctgcatccct |
| 901 | gggaggaggg agctcttaag gaagttccaa gtggatgtaa agctggatcc cgccacggcg |
| 961 | cacccgagtc tgctcttgac cgccgacctg cgcagtgtgc aggatggaga accatggagg |
| 1021 | gatgtcccca acaaccctga gcgatttgac acatggccct gcatcctggg tttgcagagc |
| 1081 | ttctcatcag ggaggcatta ctgggaggtt ctggtgggag aaggagcaga gtggggttta |
| 1141 | ggggtctgtc aagacacact gccaagaaag ggggaaacca cgccatctcc tgagaatggg |
| 1201 | gtctgggccc tgtggctgct gaaagggaat gagtacatgg tccttgcctc cccatcagtg |
| 1261 | cctcttctcc aactggaaag tcctcgctgc attgggattt tcttggacta tgaagccggt |
| 1321 | gaaatttcat tctacaatgt cacagatgga tcttatatct acacattcaa ccaactcttc |
| 1381 | tctggtcttc ttcggcctta ctttttcatc tgtgatgcaa ctcctcttat cttgccaccc |
| 1441 | acaacaatag cagggtcagg aaattgggca tccagggatc atttagatcc tgcttctgat |
| 1501 | gtaagagatg atcatctcta aaattctgtt cccaagatgc agtcctagcg tagcgaacgt |
| 1561 | tcctggagtg gggtgaagga tatcaatata ctaagtttta acagataccc catttaggtc |
| 1621 | agcacttgat tcgttgttgc tgtgaaatat gtccatggga caaaagaggg aatatgaaat |
| 1681 | atttgcatat gggaagatta tagagcataa taattttgta aatggagcaa tctcaacctc |
| 1741 | tatttctaga tcacattttc ttgatgtctt ccttcaaatt aatgaccttg gattacataa |
| 1801 | ggatttctat gcattcatta taatttgtta ttcctttcaa tatccttgta tttcaaatct |
| 1861 | tccatataag aattagacat ggcaattctt aaattgattc agaatggtct gatactattc |
| 1921 | cagtatcacc tccttaattc tgtttctcct cgttttcctg attttccttc tcattctctc |
| 1981 | cttccccgct ctgtctctct ctccctgtca ctctctctct cttgttcctt attttttgtt |
| 2041 | tcttacctct tactgtttaa cctgttgctt ccttctggat taatacattt agagccattc |
| 2101 | ctttatatgg tcacatttcc tatgacttta ctcaattact tttaaaatcc tttctattct |
| 2161 | gagactaatt tttaagaatt acaaagctca ttcttctgaa tctaatatca ctaactccta |
| 2221 | gactttttcc gttttctttg gatacacttt aagtaggaat ttatcagaat tttcattcaa |
| 2281 | ctcgttcttt aatgcagata tttactagtt ataagacctt aaggctgggt gcagtggctc |
| 2341 | acgcctgtaa tcccagcact ttgggaggct gaggcgggtg gatcacaagc tcaggagttc |
| 2401 | aagaccagcc tggccaacat ggtgaaaccc tgtctctact aaaaaaaaaa aaaaaataga |
| 2461 | aaaattagct gggcatggtg gcaggagcct gtaatcccag ctattctgga ggtggagaca |
| 2521 | ggagaattgc ttgaaccctg gaggcggagg ttgcagtgag ccaatatctc accactgtac |
| 2581 | tccagcccag tgcgagactc catctcaaaa aagaaaaaag acctcaaaca acacttctct |
| 2641 | ctctctttta gctgcttgtt atggttccta tacatggaac aattatactg gcctcactgt |
| 2701 | gttatggtaa atatttaagg tcatatttga tattgctggt ttgaattcag cttttccatt |
| 2761 | taaatacatt ataatgatga tgatgaaatc atgataatat ttaacttatt tttaaagtat |
| 2821 | attctgtacc tttccaacaa aaaggttaaa agtcattgaa ggctaacctt actgccttct |
| 2881 | ttgtatcact gtcttctaaa taattattat gtctgggtac agtggctcac gcctgtaatc |
| 2941 | ccagcacttt gggaggccga ggtgggcaga tcacgaggtc aggagattga gaccatcctg |
| 3001 | gctaacacag tgaaaccccg tctctactaa aaatacaaaa agaaattagc tgggcgtggt |
| 3061 | ggtgggtgcc tgttgtccca gctacttggg aggctgaggc aggagaatgg catgaaccca |
| 3121 | ggaggcagag cttgtagtga gccgagatcg cgccactgca ctccagccgg ggcaacagag |
| 3181 | caagactcca tctcaaaaat aaataaataa ataaataaat aaataaataa ataaataaat |
| 3241 | attacacaaa tgctaaaatg tttaaatggt aaatgcttca atgctaacca aatattaatt |
| 3301 | aatggcaaat tatttaacat tatctgataa taatctgcag aaggtttaat tttcctcctc |
| 3361 | aatttgaagt tcaagatgtt tttctcttcc agggagattt tttcgactga catctttaac |
| 3421 | ttaccttcca atcatattac taacgtagcc ttcttcctag attttttaat tgtttgatca |
| 3481 | tgagcgaaca cttctactct ctgtgataga tttgcaaaca gaggaaataa cgcatcctcg |
| 3541 | tgtccctctt cttggtgttc cacaggccat gtgtgcccta gccctcgttc atgcaaggtc |
| 3601 | tgtgtaggga aggtggactt cagctcagca acagcatccc ttcccacagg gatcaggtgg |
| 3661 | gtggcttgag ataccccttc catggggcac cacccattca gtgagacggg gaagccctgg |
| 3721 | gtgggaggga gaacacctcc acatgtcttc tactctctcc ataggatgga atgagtgtcc |
| 3781 | cagtcccagg agtatccatt tcccactgtg tagcccagta ctctggtctc actgtctctg |
| 3841 | ctgaatcctg tctcactgtg catattattg tggtttatat cagtcagtaa accaatgtga |
| 3901 | gtcttcatct cttgcattct taggttcata gttttgtgtg tctcctgtaa tgactcttct |
| 3961 | ctttcccttt ccaactcctg aaagattgcc actatttcct ctggaacttt gtttcgttac |
| 4021 | cagcaaaatc ctcgacatcc atacccgttt cctggctttc cctctccttt cctctgaatg |
| 4081 | gtagtctttt atattcagct gtccacttga catcaaaata gacattttga actcaatttg |
| 4141 | cctaaaactt acccacaaat ttctccccaa gtctctccct aactgcaaca acaaaaacca |
| 4201 | caggcttctc cctgtcactg gatggcaact ccattctttt gattgcttaa gccaggcatc |
| 4261 | cgattgagta ctttcttgat ttctccagcc cacatccagt ccatcggcaa gccctgttgg |
| 4321 | tcctaccttc agaatatgtc cggggttcag ttgtcctggc caccctgctg ctgtaaccat |
| 4381 | ggtcagaact ccatcctgcc cctctggatt atgactttcg tttcctcaca gtggtcctgc |
| 4441 | ttgggctcta ggcccttcca ctcccattct ctctacagca gctgggctga ttcctttagc |
| 4501 | acccaaggat atgttggcat cacagtgact tagataccat cacaaagacc tcccattcaa |
| 4561 | cttagagtga aagtcagaat cctcacagtg aatccccagg ccctagagga tgtgaacccc |
| 4621 | caggccctag aggatctgaa cccccatccc tcctctgatt atctctccca cccccacttc |
| 4681 | cctttgcatt ctgctccagc tgccctggcc tcatggctgg gtttccacca aagcaggcac |
| 4741 | ttcccatcac agggccattt ccccgcctgt ggcttctgct tgacattccc ttttccctga |
| 4801 | tatccccttg actcattatt ccctttcttc cttaactctt ctgagatcca gcttctcagt |
| 4861 | gataccacac agccctactc cccccagagc ccatctagag ctcacctttc cagtcgccct |
| 4921 | tgccaggctc agtggaggct ctttgttccc catacagtac gtgtcgtcgt actatattgt |
| 4981 | taggcttatt taatttatgt atgttttgcc tttttgtgct aaatgtaaac accacaaggg |
| 5041 | gaggtatctt tgtctgttga caatgataca ttcaatgttt ctcaagcacc cccaatgctg |
| 5101 | gtttgtatgt ggttatcatt caatctgtat ttgttgaatg aataaatgat tgactatgtg |
| 5161 | gagagcaaaa |

SEQ ID NO: 5 is an amino acid sequence of TRIM58.

```
    1 mawappgerl redarcpvcl dflqepvsvd cghsfclrci sefceksdga qggvyacpqc
  61 rgpfrpsgfr pnrqlaglve svrrlglgag pgarrcarhg edlsrfceed eaalcwvcda
121 gpehrthrta plqeaagsyq vklqmalelm rkeledaltq eanvgkktvi wkekvemqrq
181 rfrlefekhr gflaqeeqrq lrrleaeera tlqrlresks rlvqqskalk eladelqerc
241 qrpalglleg vrgvlsrska vtrleaenip melktaccip grrellrkfq vdvkldpata
301 hpsllltadl rsvqdgepwr dvpnnperfd twpcilglqs fssgrhywev lvgegaewgl
361 gvcqdtlprk gettpspeng vwalwllkgn eymvlaspsv pllqlesprc igifldyeag
421 eisfynvtdg syiytfnqlf sgllrpyffi cdatplilpp ttiagsgnwa srdhldpasd
481 vrddhl
```

As noted above, the erythroid-enriched nature of TRIM10 and TRIM58 establishes them as candidates for the targeting of a protein of interest for degradation in erythroid cells. In one embodiment, a binding agent that specifically binds TRIM10 or TRIM58 can be joined to a binding agent that specifically binds a protein of interest. Upon introduction to an erythroid cell, whether by expression therein or by more direct introduction, such a bispecific construct can bring the protein of interest into close proximity with the TRIM E3 ubiquitin ligase, resulting in ubiquitination of the protein of interest, thereby promoting the targeted degradation of the protein of interest in an erythroid-enriched manner. In such an embodiment, the binding agent that binds either or both of the TRIM polypeptide and the protein of interest can be, for example, antibodies or antigen-binding fragments thereof. The binding agents can be joined, for example, by a chemical or peptide linker, e.g., a flexible peptide linker, including, but not limited to a serine/glycine-rich peptide linker.

In another embodiment, a TRIM10 or TRIM58 polypeptide can be directly fused, e.g., as a fusion polypeptide, to a binding agent that specifically binds a protein of interest. In such an embodiment, upon introduction to a cell expressing the polypeptide of interest, the polypeptide of interest is brought into close proximity with the TRIM E3 ubiquitin ligase, resulting in ubiquitination of the protein of interest, thereby promoting the targeted degradation of the protein of interest. In such an embodiment, the binding agent that binds the protein of interest can be, for example, an antibody or antigen-binding fragment thereof. The binding agents can be joined, for example, by a chemical or peptide linker, e.g., a flexible peptide linker, including, but not limited to a serine/glycine-rich peptide linker.

### Erythroid Targets for Degradation

Any polypeptide expressed in an erythroid cell, whether erythroid specific, erythroid enriched, or simply expressed in an erythroid lineage cell, can be target for proteasome-mediated degradation using constructs as described herein. Non-limiting examples of erythroid-enriched factors include LRF, ZNF410, and BLC11A.

### LRF

Leukemia/Lymphoma-Related Factor (LRF), also known as Zinc Finger and BTB Domain Containing 7A (ZBTB7A), Factor binding IST protein 1 (FBI-1), or Pokemon is a member of the POK family of transcription factors that possesses a POZ-domain at the N-terminus and Krüppel-like zinc finger at the c-terminus. See, e.g., Gupta et al., Cancer Lett. 483, 22-34 (2020), which is incorporated herein by reference in its entirety. LRF is involved in several fundamental biological processes including cell proliferation, differentiation, and development. It also participates in hematopoiesis and erythrocyte maturation, adipogenesis, chondrogenesis, cellular metabolism and alternative splicing of BCLXL, DNA repair, development of oligodendrocytes, osteoclast, and the unfolded protein response. LRF also negatively regulates SMAD4-DNA complex. Sequences of LRF are known for a number of species, e.g., human LRF/ZBTB7A (NCBI GeneID: 51341), mRNA (e.g., LRF variant 2 NCBI Ref Seq NM_001317990.2, SEQ ID NO: 6) and polypeptide (e.g., LRF: NCBI Ref Seq: NP_001304919.1, SEQ ID NO: 7). Antibodies for specific detection or binding to LRF/ZBTB7A are available, for example, from R&D Systems, see Human/Mouse ZBTB7A/Pokemon Antibody (clone 466407) MAB3496; from Origen, see Pokemon (ZBTB7A) Mouse Monoclonal Antibody (clone BC17-2.9F), AM32711PU—N; from Novusbio, see ZBTB7A/Pokemon Antibody (clone 3T2J1) NBP3-15914; from Abcam, see Anti-ZBTB7A antibody (EPR13178B) ab175918; and from Invitrogen, see Pokemon (LRF) monoclonal antibody (13E9) 14-3309-82. LRF can refer to human LRF, including naturally occurring variants and alleles thereof. For example, SEQ ID NO: 6 is a transcript variant 2, mRNA of human LRF. Note: U's replaced with T's in this representation.

| | |
|---|---|
| 1 | actgccgcct cccggcccct cggagggagc cagcccagcc gcagccgccg ccaccgccgc |
| 61 | cgccggggcc gggccccctc gccgctgccc cgggaaggag gtctcggcgc ggaagatggc |
| 121 | cggcggcgtg gacggcccca tcgggatccc gttccccgac cacagcagcg acatcctgag |
| 181 | tgggctgaac gagcagcgga cgcagggcct gctgtgcgac gtggtgatcc tggtggaggg |
| 241 | ccgcgagttc cccacgcacc gctcggtgct ggccgcctgc agccagtact tcaagaagct |
| 301 | gttcacgtcg ggcgccgtgg tggaccagca gaacgtgtac gagatcgact tcgtcagcgc |
| 361 | cgaggcgctc accgcgctca tggacttcgc ctacacggcc acgctcaccg tcagcacagc |
| 421 | caacgtgggt gacatcctca gcgccgcccg cctgctggag atccccgccg tgagccacgt |
| 481 | gtgcgccgac ctcctggacc ggcagatcct gcgggccgac gcgggcgccg acgccgggca |
| 541 | gctggacctt gtagatcaaa ttgatcagcg caacctcctc cgcgccaagg agtacctcga |
| 601 | gttcttccag agcaacccca tgaacagcct gccccccgcg gccgccgccg ccgctgccag |
| 661 | cttcccgtgg tccgcctttg gggcgtccga tgatgacctg gatgccacca aggaggccgt |
| 721 | ggccgccgct gtggccgccg tggccgcggg cgactgcaac ggcttagact tctatgggcc |
| 781 | gggccccccg gccgageggc ccccgacggg ggacggggac gagggcgaca gcaacccggg |
| 841 | tctgtggcca gagcgggatg aggacgcccc caccgggggt ctctttccgc cgccggtggc |
| 901 | cccgccggcc gccacgcaga acggccacta cggccgcggc ggagaggagg aggccgcctc |
| 961 | gctgtcggag gcggcccccg agccgggcga ctctccgggc ttcctgtcgg gagcggccga |
| 1021 | gggcgaggac ggggacgggc ccgacgtgga cgggctggcg gccagcacgc tgctgcagca |
| 1081 | gatgatgtca tcggtgggcc gggcgggggc cgcggcgggg gacagcgacg aggagtcgcg |
| 1141 | ggccgacgac aagggcgtca tggactacta cctgaagtac ttcagcggcg cccacgacgg |
| 1201 | cgacgtctac ccggcctggt cgcagaaggt ggagaagaag atccgagcca aggccttcca |
| 1261 | gaagtgcccc atctgcgaga aggtcatcca gggcgccggc aagctgccgc gacacatccg |
| 1321 | cacccacacg ggcgagaagc cctacgagtg caacatctgc aaggtccgct tcaccaggca |
| 1381 | ggacaagctg aaggtgcaca tgcggaagca cacgggcgag aagccgtacc tgtgccagca |
| 1441 | gtgcggcgcc gcctttgccc acaactacga cctgaagaac cacatgcgcg tgcacacggg |
| 1501 | cctgcgcccc taccagtgcg acagctgctg caagaccttc gtccgctccg accacctgca |
| 1561 | cagacacctc aagaaagacg gctgcaacgg cgtcccctcg cgcegcggcc gcaagccccg |
| 1621 | cgtccggggc ggggcgcccg accccagccc gggggccacc gcgacccccg gcgcccccgc |
| 1681 | ccagcccagc tcccccgacg cccggcgcaa cggccaggag aagcacttta aggacgagga |
| 1741 | cgaggacgag gacgtggcca gccccgacgg cttgggccgg ttgaatgtag cgggcgccgg |
| 1801 | tggaggaggt gacagcggag gtggccccgg ggccgccacc gacggtaact tcacagccgg |
| 1861 | actcgcctaa aaaccaaaaa gagaaaacag aaacccgaga aagagagaga cagagagaga |
| 1921 | gaaaaaaaat cacccaccac ccccccaaaa acacaaaaaa agaaaatcta tctatataca |
| 1981 | gatatctata tctatatata tatatacaga tatatatata tgacgcgtca cagaatctag |
| 2041 | ggtagcgctt tctcagattt ccctcctttc tgacgttttt ctccctccgc aggggccccg |
| 2101 | gccctccctg gctccccttc cccccaccac cccatcgctg ggtttcgggg cttggtttgg |
| 2161 | ggttttttgt aggacacaag gaatccgaga ccccgcacag ccccctgggc acccggcatg |
| 2221 | gggcctgggg cccgatccga ggccctgggc tggggggagg gtagacgtgg gggcgctggg |
| 2281 | gggggactgg ggtgggcttt taatttcctc ccctcgctgg tttctatgag tctttcagac |
| 2341 | aagaccttaa atgatttctg tctgctctga gcggacgtta aaatgggccc ccgtcccccg |
| 2401 | acccgcaccc tccttcctca gggcacttac taagggaggg gtctccctct ccatctcccc |
| 2461 | agtggcctcc ccgcctccaa ccctgcctgc ggcctccccc cgtcgcccac cccacgtctc |
| 2521 | ctggccactg agacacaaac ctatttattt ctaggcctgg agaaaggaga tcggactggg |
| 2581 | gttcccggtg gggcgccagg atggctcctg ggggtgctcc tgccgccttc cttcacggca |
| 2641 | cttacaaccg gcgggacccc cagggaccac ccctcagggc gcccccccac ccccgcccgg |
| 2701 | tccacctaga cccccacgtt tggagattca aaacttctgt cttcgtcctc tcccccgagc |
| 2761 | cccctctccc aaatttttaa agcacttttt agattttttt ttctctttcc tccttaaaaa |
| 2821 | caaaatttat atatagatat atatatatat ataaataata tacttttcct cagaggagca |
| 2881 | ggcaacagtg tgggataaac agagtcacga tcagaggaac cccagggtct ggtgatggca |
| 2941 | gggatggggg gagagagaga aaatccacaa attccaatgt cacaaaagca ataaaacaaa |
| 3001 | ctagaaaaaa aaaaggtttt acaaaatgaa aggaaggaaa aaaaaaaagg caaccaacca |
| 3061 | cattagaagt cttggcactt tgtaacggaa cgggtactac actttatctt aattcttaat |
| 3121 | ttaaaaacat gtttacaagt tacaaccaac ttctatgaaa agttgaaaag acaaaaaaaa |
| 3181 | aaaaaaaaag cgagcgagag agagagcgag agagagagcg agagcagaag aaattcctaa |
| 3241 | aagtcgattt atttttgtac aaaataataa aaaaaaaaac ccaccacaaa cgtagaatcc |
| 3301 | acttctgttc cccaaaaagc gagaaggggg gttcaggagg aagccatcgc aggggacctg |
| 3361 | ggagacgccc cgaggtgttt gtgcttcacc cccagacgtc agcctcgaag gcaggactgt |
| 3421 | ggggtgttcg tgctgtgttc cccccgctcc ccctttctgt cccctttttt ggttctgacg |
| 3481 | tgaagaggtc ttagcgcccg cttctgtcca cggggtctct ccttcctcct ccctagctca |
| 3541 | gggatgggcc ttccagccgg agcaccccga tccccatccg gcacccccca atcccccaac |
| 3601 | acgcctgtcc ctcccgcatg gccaccaagg agctggacct tggatgcgcc taccctgctg |
| 3661 | aggtgggtga caggggcccc ccacctccag ggccttagaa ccaccgcccc tctccccacc |
| 3721 | ccaggcaccc ctctttttac tcaaaggcac tgactgtaat ccagggggac tgggacctgc |
| 3781 | ctccccccaa cctctggctc ccacaaggcc cggtgttgac cgagccacag gccacggaca |
| 3841 | ggggccgggg ttggggagac tatgtcgcca gatgccagga cgccctcacc ccgtttgcat |
| 3901 | atgcaatgct agcatgggac cccgaaaata gacgctctgc tgcactgaga cttcttgtca |
| 3961 | atgcccaacc ggcggggggg tgtctccctg cccccgaccc ccccataccc ccttctctgt |
| 4021 | gacacacaca tcttctcgtc tctttttctt tcattgttaa agggaagctt tttaagaagg |
| 4081 | caattttcat attgtttcta caggatggtt ttggttccct tcccttccca ccccccctta |
| 4141 | agcctgtcag ccccctccaa atgtctcagg atcccccctc tcccctgggg ctgggtgaca |
| 4201 | gcaccccggc tgcgttcaca ccccagtgtc acagggcgag ctgttctgga gagaaaacca |
| 4261 | tctgtcgtgg ctgagcgggg agcttgaaca cccaggccag ggacacccct ccccagctcc |
| 4321 | cagagaggcc ccctgagggg tgagccctct ttccaccttc ccctatccat gcaccccctc |
| 4381 | gcaataaaac caactctaaa atcacagctg tcgtcctagc cagtgggggc gaccggactt |
| 4441 | ggggggtgga gccctctggg acttccgtag gaacaagggc tgcggcccac cgcgacactt |
| 4501 | acacagacct cggggattgc actaaaccct cgttcctagc tccgcactca gcttcgcctg |
| 4561 | tcctgcccgc ccactttgcc ttaactaccc gcccgtcctg ggggccacag cctctgcatg |
| 4621 | ggcccagagc cgggaccccc ccagcccage cccgccctcc ccagactccg cgcaatcaca |
| 4681 | tactgtatat agacgtgaat cgattttatt tttattcttt aaattaaggt cgtgataaag |
| 4741 | tgttgccaaa gatacctgct gaattctcgc gtttcaggaa acaaacaaac aaaaaaaaat |
| 4801 | gatatttgag gagggtcgtg ttgactccat atgaaaggac acagctcaaa gcttttttgt |
| 4861 | ttggttgttt ggggtttttt gtgttttctt tttttggggt gttttttttt taactgcctg |
| 4921 | gtacaaaaaa aaaaagagaa aaaaaaaaaa gaaaaacaat gcgaaattgt tatttccatt |
| 4981 | ctcatggtga agttgcgtgg acgcgtgtgt gcgtgtgtgc aagagagcgg gagtgaggtc |
| 5041 | caggctgggg ttggggggct tcaggcgggg gcgcccgggg gccggggagg tggccgggcc |
| 5101 | ggagcccccg tctgcagtgc cccccagcct gccgggccca ggagagagag agaagcatct |
| 5161 | ttgctactag ctgttgctgc tacctgcctc tgccccccga cgccccccgc cttttgagat |
| 5221 | taaggaaaaa aaaaaaaagt caaaaaagtt tttaaaaatg aaaaaaaaaa attataaacc |
| 5281 | agtgaatgta aaatgccgga gcaggcccgg cctggcatgg gtgtggacct gcagccaggc |
| 5341 | aggctcgagc gggcgatacc aaagtctgcc cccccaccat tgtggccatg cagtcctgtc |
| 5401 | actgtctttt tgcttccttc cgaggggggt cccccagcct cttccagggt cttcccctgg |
| 5461 | aagtgggcgg ctgcagggaa ggtgggggac aggggtcttt gcacgattca gaccccgggg |
| 5521 | ccgtggcagg agcggtcacc tcacaggtgg tgacactgag gcaggggcct cggggtgccc |
| 5581 | cctcccgccc ggcaaccaga atggttggag gcaagacaga gagaatgaaa ggaaaaacag |
| 5641 | aagaaaaaaa aatattaaaa accaacaaaa aaagcaaaaa tcctattttt tgagaaagaa |
| 5701 | agatatttat atttgcagtt ttattttaaa aagttattta agttgaagca gccttcctgg |
| 5761 | aggtgggggg gggggggtgg tgggtggctg gcgcaggacg ggtcaggggc ctggaggctg |
| 5821 | ggggtgcccc aggagctaca acctcagagt taagactagc tcgcattaaa tacatagatt |
| 5881 | tacggggggg gggggggggg gccgggccca gggggtggag ggggccaggg agacccccat |
| 5941 | ccctcgccgg ggctgcctgg aggctgtgga ccaggatccg atgcccaggt cccgcccccc |
| 6001 | accccacccc aggcccagaa tcgaggtgcc ttggactttg gaggggccag gcctggtgaa |
| 6061 | tggggggcgg ggcggcgccc tcagggtaca gagcacagac agatagacat tccagagact |
| 6121 | gtattgagag tctttataaa gtgtgggaga tttaaaaaaa aaaaaaactg ataaaaatgc |
| 6181 | actttttggg agtggggagg gagaagcttt aaaagtaata aaaaacaaac aaaaacacaa |
| 6241 | aagatgaaaa aacaaaaaaa ttcatttttc ttgtacataa aaaaaaaaaa agaaccacta |
| 6301 | aacgcagcct gttacgacc |

SEQ ID NO: 7 is an amino acid sequence of human LRF.

| | |
|---|---|
| 1 | maggvdgpig ipfpdhssdi lsglneqrtq gllcdvvilv egrefpthrs vlaacsqyfk |
| 61 | klftsgavvd qqnvyeidfv saealtalmd faytatltvs tanvgdilsa arlleipavs |
| 121 | hvcadlldrq ilaadagada goldlvdqid qrallrakey leffqsnpmn slppaaaaaa |
| 181 | asfpwsafga sdddldatke avaaavaava agdengldfy gpgppaerpp tgdgdegdsn |
| 241 | pglwperded aptgglfppp vappaatqng hygrggeeea aslseaapep gdspgflsga |
| 301 | aegedgdgpd vdglaastll qqmmssvgra gaaagdsdee sraddkgvmd yylkyfsgah |
| 361 | dgdvypawsq kvekkiraka fqkcpicekv iqgagklprh irthtgekpy ecnickvrft |
| 421 | rqdklkvhmr khtgekpylc qqcgaafahn ydlknhmrvh tglrpyqcds ccktfvrsdh |
| 481 | lhrhlkkdgc ngvpsrrgrk prvrggapdp spgatatpga paqpsspdar rngqekhfkd |
| 541 | edededvasp dglgrinvag aggggdsggg pgaatdgnft agla |

In some embodiments, LRF can be targeted for degradation in erythroid cells via TRIM10 or TRIM58 in a manner directly analogous to that described herein for targeting of BCL11A in erythroid cells. Briefly, in one embodiment, a binding agent, e.g., an antibody or antigen-binding fragment thereof specific for TRIM10 or TRIM58 and a binding agent, e.g., an antibody or antigen-binding fragment thereof specific for LRF can be fused, optionally via a peptide linker. In another embodiment, TRIM10 or TRIM58 polypeptide itself can be fused to a binding agent, e.g., an antibody or antigen-binding fragment thereof specific for LRF. Introduction of either or both of these constructs to a cell, e.g., an erythroid cell, for example as discussed herein for BCL11A-targeting constructs, can target LRF for degradation in those cells.

### ZNF410

Zinc Finger Protein 410 (ZNF410), also known as APA-1 is a transcription factor that contains five tandem canonical C2H2 zinc fingers. See, e.g., Lan et al., Mol Cell. 21:239-254, (2021), which is incorporated herein by reference in its entirety. ZNF410 binds to the sequence motif 5′-CATCCCATAATA-3′ (SEQ ID NO: 66) in regulatory elements and modulates transcription of target genes. In erythroid cells, ZNF410 directly binds and transcriptionally activates CHD4, a component of the NuRD complex, which leads to repression of fetal globin genes HBG1 and HBG2. Sequences of ZNF410 are known for a number of species, e.g., human ZNF410 (NCBI GeneID: 57862), mRNA (e.g., ZNF410 Isoform a: NCBI Ref Seq NM_001242924.2, SEQ ID NO: 8) and polypeptide (e.g., ZNF410 Isoform a: NCBI Ref Seq: NP_001229853.1, SEQ ID NO: 9). Antibodies for specific detection of or binding to ZNF410 are available, for example, from Creative Biolabs, see Mouse Anti-ZNF410 Recombinant Antibody (clone 1C7) CBMAB-0827-LY; Mouse Anti-ZNF410 Recombinant Antibody (clone 1A12) CBMAB-0826-LY; and Anti-ZNF410 monoclonal antibody (clone DCABH-17601. ZNF410 can refer to human ZNF410, including naturally occurring variants and alleles thereof. For example, SEQ ID NO: 8 is an mRNA sequence of human ZNF410. Note: U's replaced with T's in this representation.

| | |
|---|---|
| 1 | gtgtgtggac ggaattcggg accgactgac ggccggccgg cttcccggaa ctggaaggtt |
| 61 | acattgatta cccacctagt acaacatctt acgggaagag catagtattt cctagaggaa |
| 121 | tatgaacata acaggaaggt atcattggct ctgaattaaa tttgaacttg tcccctgaat |
| 181 | agctacaggt tttggaagct gaatcaatgt tatcagatga gttagaatcc aaaccagagc |
| 241 | tcctggtaca gtttgttcag aatacgtcca tcccattggg acaggggctt gtagaatcag |
| 301 | aagctaaaga tattacttgc ttgtccctcc ttcccgtgac tgaagcctca gaatgcagtc |
| 361 | ggctaatgtt accagaacct gttccatgga gagaagagga tggaaagtca ggctgcagtg |
| 421 | acttgaatga tactacaaat cattctaact cctccaagga ggtcccttcc tcagctgttt |
| 481 | tgagaagcct tcgggtgaat gtgggtccag acggagagga gacgagagct cagactgtac |
| 541 | agaaatcccc ggagtttttg tccacttcag agtcttctag cttgttgcaa gatctacagc |
| 601 | caagtgatag cacttctttt attcttctta acctaacaag agcaggtctg ggctcttcag |
| 661 | ctgagcactt agtgtttgta caggatgagg cagaagattc agggaatgat ttcctctcca |
| 721 | gtgagagcac agacagtagc attccatggt tcctccgggt tcaggagttg gcccatgaca |
| 781 | gtttgattgc tgctactcgt gcacaactgg caaagaatgc aaaaaccagc agcaatggag |
| 841 | aaaatgtcca ccttggttct ggtgatgggc agtcaaaaga ttctgggccc cttcctcaag |
| 901 | tggaaaagaa gctcaagtgt acagttgaag gttgtgaccg gacatttgta tggccagctc |
| 961 | actttaaata ccacctcaag actcatcgaa atgaccgctc cttcatctgt cctgcagaag |
| 1021 | gttgtgggaa aagcttctat gtgctgcaga ggctgaaggt gcacatgagg acccacaatg |
| 1081 | gagagaagcc ctttatgtgc catgagtctg gctgtggtaa gcagtttact acagctggaa |
| 1141 | acctgaagaa ccaccggcgc atccacacag gagagaaacc tttcctttgt gaagcccaag |
| 1201 | gatgtggccg ttcctttgct gagtattcta gcctccgaaa acatctggtg gttcactcag |
| 1261 | gagagaagcc tcatcagtgc caagtctgtg ggaagacctt ctctcagagt ggaagcagga |
| 1321 | atgtgcatat gagaaagcat cacctgcagc tgggagcagc tgggagtcaa gagcaggagc |
| 1381 | aaactgctga gccactaatg ggcagtagtt tgcttgaaga ggcttcagta cccagtaaaa |
| 1441 | acctggtgtc tatgaattcc cagcccagcc ttggtggaga gtccttgaac ctaccaaata |
| 1501 | ccaattctat cctgggagtt gatgatgagg tgcttgctga aggatcccca cgttccctgt |
| 1561 | cttcagtgcc tgatgtgaca catcacctgg tgaccatgca gtcagggagg caatcatatg |
| 1621 | aagtttctgt cttaactgca gtaaatccac aagagagtct cgctccgttg cccaggctgg |
| 1681 | agtgcagtgg tgcgttctcg gctcactgca acctctgcct cccaggttca agcgattctc |
| 1741 | ctgcctcagc ctcctgagta gctgctacta cagttactaa accaaggaga tttaactgaa |
| 1801 | agacggacat gagcgtgggt gctgactcct ggaagagcaa ctctatctga tctcaaaatg |
| 1861 | cgtatactgg gaacaggatg ccttagccca caacagaacc agaatgaatc tttgaaggca |
| 1921 | caagactctg cttttgccac tcttcctctt tcctggtata gaagatggat gtaggagagc |
| 1981 | ttcttttcta actaccatct gatcagacaa ggaatgaagc aatgactgtg ggctgggaaa |
| 2041 | ctgtacctac ctctcttccc actgcaaatt tctgggatag accaaaagtg aatttgatta |
| 2101 | tgtgttggct gaagttcttc attctgactg ttgaggggag gttttccttt gaagagtttt |
| 2161 | catcccagac tcagctgtct tttcacatgg atgaaataat tcctgctacc aacaacagag |
| 2221 | cttcaccagg aagttgagtt ttcaagatgc cttgttgctt tgaagaaggg agtgatgtca |
| 2281 | attctcttgt tacattctcc ctttagcaac ctgagtaaga gactctctgc cactgggctg |
| 2341 | caaaaaaata aattacttga atctcccctt ggcccaggct gaggtactat cttgtcctat |
| 2401 | acacttctac ttggtcactt tgctttcttc gttaatggaa catacagtag catgttaaga |
| 2461 | gggatttcat gtttttgttt ttttaaagtt aaaaattaca tgatgcagag attcaggttt |
| 2521 | tcctttaaat aaacaaaaca gcccagtcag ttctttggct cttgttttat acaaaatttg |
| 2581 | ttttcttaga gattaagaat ttaatctttc cctttaaaat agtgtattga tttaccctta |
| 2641 | ggattccata ccagtaaaac tgaaccgagg agtcttagga aacaacaaga acatcttcat |
| 2701 | ttcttaagcc caggtgatag ttactctgtc accaccaaaa aagacgcatc tgagaaaatg |
| 2761 | gcaataaaaa cagatacttc tgaatttttc ca |

SEQ ID NO: 9 is an amino acid sequence of human ZNF410.

```
    1 mlsdeleskp ellvqfvqnt siplgqglve seakditcls llpvteasec srlmlpepvp
  61 wreedgksgc sdlndttnhs nsskevpssa vlrslrvnvg pdgeetraqt vqkspeflst
121 sesssllqdl qpsdstsfil Inltraglgs saehlvfvqd eaedsgndfl ssestdssip
181 wflrvqelah dsliaatraq laknaktssn genvhlgsgd gqskdsgplp qvekklkctv
241 egcdrtfvwp ahfkyhlkth rndrsficpa egcgksfyvl qrlkvhmrth ngekpfmche
301 sgcgkqftta gnlknhrrih tgekpflcea qgcgrsfaey sslrkhlvvh sgekphqcqv
361 cgktfsqsgs rnvhmrkhhl qlgaagsqeq eqtaeplmgs slleeasvps knlvsmnsqp
421 slggeslnlp ntnsilgvdd evlaegsprs lssvpdvthh lvtmqsgrqs yevsvltavn
481 pqeslaplpr lecsgafsah cnlclpgssd spasas
```

In some embodiments, ZNF410 can be targeted for degradation in erythroid cells via TRIM10 or TRIM58 in a manner directly analogous to that described herein for targeting of BCL11A in erythroid cells. Briefly, in one embodiment, a binding agent, e.g., an antibody or antigen-binding fragment thereof specific for TRIM10 or TRIM58 and a binding agent, e.g., an antibody or antigen-binding fragment thereof specific for ZNF410 can be fused, optionally via a peptide linker. In another embodiment, TRIM10 or TRIM58 polypeptide itself can be fused to a binding agent, e.g., an antibody or antigen-binding fragment thereof specific for ZNF410. Introduction of either or both of these constructs to a cell, e.g., an erythroid cell, for example as discussed herein for BCL11A-targeting constructs, can target ZNF410 for degradation in those cells.

### BCL11A

BCL11A (B-cell lymphoma/leukemia 11A), also known as Evi9, CTIP1, or ZNF856 is a Kruppel-like sequence specific C2H2 type zinc-finger transcription factor located on chromosome 2. See, e.g., Liu et al., Cell. 173, 430-442 (2018) and Satterwhite et al., Blood. 98, 3413-3420 (2001), which are incorporated herein by reference in their entireties. BCL11A functions mainly as a transcriptional repressor that is involved in brain and hematopoietic system development, as well as fetal-to-adult hemoglobin switching. Sequences of BCL11A are known for a number of species, e.g., human BCL11A (NCBI GeneID: 53335), mRNA (e.g., BCL11A isoform 1 NCBI Ref Seq NM_00022893.4, SEQ ID NO: 10) and polypeptide (e.g., BCL11A isoform 1: NCBI Ref Seq: NP_075044.2, SEQ ID NO: 11). BCL11A can refer to human BCL11A, including naturally occurring variants and alleles thereof. For example, SEQ ID NO: 10 is a mRNA sequence for Isoform 1 of human BCL11A. Note: U's replaced with T's in this representation.

| | |
|---|---|
| 1 | gtctctgtcc atccagactc ctgacgttca agttcgcagg gacgtcacgt ccgcacttga |
| 61 | acttgcagct caggggggct tttgccattt ttttcatctc tctctctctc tctccctcta |
| 121 | tctctcttct ctctctctcc ctcttttttt tttttttttt tttttttttt ttgcttaaaa |
| 181 | aaaagccatg acggctctcc cacaattcat cttccctgcg ccatctttgt attatttcta |
| 241 | atttattttg gatgtcaaaa ggcactgatg aagatatttt ctctggagtc tccttctttc |
| 301 | taacccggct ctcccgatgt gaaccgagcc gtcgtccgcc cgccgccgcc gccgccgccg |
| 361 | ccgccgcccg ccccgcagcc caccatgtct cgccgcaagc aaggcaaacc ccagcactta |
| 421 | agcaaacggg aattctcgcc cgagcctctt gaagccattc ttacagatga tgaaccagac |
| 481 | cacggcccgt tgggagctcc agaaggggat catgacctcc tcacctgtgg gcagtgccag |
| 541 | atgaacttcc cattggggga cattcttatt tttatcgagc acaaacggaa acaatgcaat |
| 601 | ggcagcctct gcttagaaaa agctgtggat aagccacctt ccccttcacc aatcgagatg |
| 661 | aaaaaagcat ccaatcccgt ggaggttggc atccaggtca cgccagagga tgacgattgt |
| 721 | ttatcaacgt catctagagg aatttgcccc aaacaggaac acatagcaga taaacttctg |
| 781 | cactggaggg gcctctcctc ccctcgttct gcacatggag ctctaatccc cacgcctggg |
| 841 | atgagtgcag aatatgcccc gcagggtatt tgtaaagatg agcccagcag ctacacatgt |
| 901 | acaacttgca aacagccatt caccagtgca tggtttctct tgcaacacgc acagaacact |
| 961 | catggattaa gaatctactt agaaagcgaa cacggaagtc ccctgacccc gcgggttggt |
| 1021 | atcccttcag gactaggtgc agaatgtcct toccagccac ctctccatgg gattcatatt |
| 1081 | gcagacaata acccctttaa cctgctaaga ataccaggat cagtatcgag agaggcttcc |
| 1141 | ggcctggcag aagggcgctt tccacccact ccccccctgt ttagtccacc accgagacat |
| 1201 | cacttggacc cccaccgcat agagcgcctg ggggcggaag agatggccct ggccacccat |
| 1261 | cacccgagtg cctttgacag ggtgctgcgg ttgaatccaa tggctatgga gcctcccgcc |
| 1321 | atggatttct ctaggagact tagagagctg gcagggaaca cgtctagccc accgctgtcc |
| 1381 | ccaggccggc ccagccctat gcaaaggtta ctgcaaccat tccagccagg tagcaagccg |
| 1441 | cccttcctgg cgacgccccc cctccctcct ctgcaatccg cccctcctcc ctcccagccc |
| 1501 | ccggtcaagt ccaagtcatg cgagttctgc ggcaagacgt tcaaatttca gagcaacctg |
| 1561 | gtggtgcacc ggcgcagcca cacgggcgag aagccctaca agtgcaacct gtgcgaccac |
| 1621 | gcgtgcaccc aggccagcaa gctgaagcgc cacatgaaga cgcacatgca caaatcgtcc |
| 1681 | cccatgacgg tcaagtccga cgacggtctc tccaccgcca gctccccgga acccggcacc |
| 1741 | agcgacttgg tgggcagcgc cagcagcgcg ctcaagtccg tggtggccaa gttcaagagc |
| 1801 | gagaacgacc ccaacctgat cccggagaac ggggacgagg aggaagagga ggacgacgag |
| 1861 | gaagaggaag aagaggagga agaggaggag gaggagctga cggagagcga gagggtggac |
| 1921 | tacggcttcg ggctgagcct ggaggcggcg cgccaccacg agaacagctc gcggggcgcg |
| 1981 | gtcgtgggcg tgggcgacga gagccgcgcc ctgcccgacg tcatgcaggg catggtgctc |
| 2041 | agctccatgc agcacttcag cgaggccttc caccaggtcc tgggcgagaa gcataagcgc |
| 2101 | ggccacctgg ccgaggccga gggccacagg gacacttgcg acgaagactc ggtggccggc |
| 2161 | gagtcggacc gcatagacga tggcactgtt aatggccgcg gctgctcccc gggcgagtcg |
| 2221 | gcctcggggg gcctgtccaa aaagctgctg ctgggcagcc ccagctcgct gagccccttc |
| 2281 | tctaagcgca tcaagctcga gaaggagttc gacctgcccc cggccgcgat gcccaacacg |
| 2341 | gagaacgtgt actcgcagtg gctcgccggc tacgcggcct ccaggcagct caaagatccc |
| 2401 | ttccttagct tcggagactc cagacaatcg ccttttgcct cctcgtcgga gcactcctcg |
| 2461 | gagaacggga gtttgcgctt ctccacaccg cccggggagc tggacggagg gatctcgggg |
| 2521 | cgcagcggca cgggaagtgg agggagcacg ccccatatta gtggtccggg cccgggcagg |
| 2581 | cccagctcaa aagagggcag acgcagcgac acttgtgagt actgtgggaa agtcttcaag |
| 2641 | aactgtagca atctcactgt ccacaggaga agccacacgg gcgaaaggcc ttataaatgc |
| 2701 | gagctgtgca actatgcctg tgcccagagt agcaagctca ccaggcacat gaaaacgcat |
| 2761 | ggccaggtgg ggaaggacgt ttacaaatgt gaaatttgta agatgccttt tagcgtgtac |
| 2821 | agtaccctgg agaaacacat gaaaaaatgg cacagtgatc gagtgttgaa taatgatata |
| 2881 | aaaactgaat agaggtatat taatacccct ccctcactcc cacctgacac cccctttttc |
| 2941 | accactcccc ttccccatcg ccctccagcc ccactccctg taggattttt ttctagtccc |
| 3001 | atgtgattta aacaaacaaa caaacaaaca gaagtaacga agctaagaat atgagagtgc |
| 3061 | ttgtcaccag cacacctgtt ttttttcttt ttctttttct tttttctttt tccttttttt |
| 3121 | tttttttcct ttatgttctc accgtttgaa tgcatgatct gtatggggca atactattgc |
| 3181 | attttacgca aactttgagc ctttctcttg tgcaataatt tacatgttgt gtatgttttt |
| 3241 | ttttaaactt agacagcatg tatggtatgt tatggctatt ttaaattgtc cctaattcgt |
| 3301 | tgctgagcaa acatgttgct gtttccagtt ccgttctgag agaaaaagag agagagagag |
| 3361 | aaaaagacca tgctgcatac attctgtaat acatatcatg tacagtttta ttttataacg |
| 3421 | tgaggaggaa aaacagtctt tggattaacc ctctatagac agaatagata gcactgaaaa |
| 3481 | aaaatctcta tgagctaaat gtctgtctct aaagggttaa atgtatcaat tggaaaggaa |
| 3541 | gaaaaaaggc cttgaattga caaattaaca gaaaaacaga acaagtttat tctatcattt |
| 3601 | ggttttaaaa tatgagtgcc ttggatctat taaaaccaca tcgatggttc tttctacttg |
| 3661 | ttataaactt gtagcttaat tcagcattgg gtgaggtaat aaaccttagg aactagcata |
| 3721 | taattctata ttgtatttct cacaacaatg gctacctaaa aagatgaccc attatgtcct |
| 3781 | agttaatcat catttttcct ttagtttaat tttataaaca aaactgatta taccagtata |
| 3841 | aaagctactt tgctcctggt gagagcttaa aagaaatggg ctgttttgcc caaagtttta |
| 3901 | ttttttttaa acaatgatta aattgaatgt gtaatgtgca aaagccctgg aacgcaatta |
| 3961 | aatacactag taaggagttc attttatgaa gatatttgct ttaataatgt ctttttaaaa |
| 4021 | atactggcac caaaagaaat agatccagat ctacttggtt gtcaagtgga caatcaaatg |
| 4081 | ataaacttta agaccttgta taccatattg aaaggaagag gctgacaata aggtttgaca |
| 4141 | gaggggaaca gaagaaaata atatgattta ttagcacaac gtggtactat ttgccattta |
| 4201 | aaactagaac aggtatataa gctaatattg atacaatgat gattaactat gaattcttaa |
| 4261 | gacttgcatt taaatgtgac attcttaaaa aaagaagaga aagaatttta agagtagcag |
| 4321 | tatatatgtc tgtgctccct aaaagttgta cttcatttct tttccataca ctgtgtgcta |
| 4381 | tttgtgttaa catggaagag gattcattgt ttttattttt atttttttaa ttttttcttt |
| 4441 | tttattaagc tagcatctgc cccagttggt gttcaaatag cacttgactc tgcctgtgat |
| 4501 | atctgtatct tttctctaat cagagataca gaggttgagt ataaaataaa cctgctcaga |
| 4561 | taggacaatt aagtgcactg tacaattttc ccagtttaca ggtctatact taagggaaaa |
| 4621 | gttgcaagaa tgctgaaaaa aaattgaaca caatctcatt gaggagcatt ttttaaaaac |
| 4681 | taaaaaaaaa aaaactttgc cagccattta cttgactatt gagcttactt acttggacgc |
| 4741 | aacattgcaa gcgctgtgaa tggaaacaga atacacttaa catagaaatg aatgattgct |
| 4801 | ttcgcttcta cagtgcaagg atttttttgt acaaaacttt tttaaatata aatgttaaga |
| 4861 | aaaatttttt ttaaaaaaca cttcattatg tttagggggg aactgcattt tagggttcca |
| 4921 | ttgtcttggt ggtgttacaa gacttgttat ccatttaaaa atggtagtgg aaattctatg |
| 4981 | ccttggatac acaccgctct tcaggttgta aaaaaaaaaa acatacattg gggaaaggtt |
| 5041 | taagattata tagtacttaa atataggaaa atgcacactc atgttgattc ctatgctaaa |
| 5101 | atacatttat ggtctttttt ctgtatttct agaatggtat ttgaattaaa tgttcatcta |
| 5161 | gtgttaggca ctatagtatt tatattgaag cttgtatttt taactgttgc ttgttctctt |
| 5221 | aaaaggtatc aatgtacctt ttttggtagt ggaaaaaaaa aagacaggct gccacagtat |
| 5281 | atttttttaa tttggcagga taatatagtg caaattattt gtatgcttca aaaaaaaaaa |
| 5341 | aaagagagaa acaaaaaagt gtgacattac agatgagaag ccatataatg gcggtttggg |
| 5401 | ggagcctgct agaatgtcac atggatggct gtcatagggg ttgtacatat ccttttttgt |
| 5461 | tcctttttcc tgctgccata ctgtatgcag tactgcaagc taataacgtt ggtttgttat |
| 5521 | gtagtgtgct ttttgtccct ttccttctat caccctacat tccagcatct taccttcata |
| 5581 | tgcagtaaaa gaaagaaaga aaaaaaaagg aaaaaaaaaa aaaaaccaat gttttgcagt |
| 5641 | ttttttcatt gccaaaaact aaatggtgct ttatatttag attggaaaga atttcatatg |
| 5701 | caaagcatat taaagagaaa gcccgcttta gtcaatactt ttttgtaaat ggcaatgcag |
| 5761 | aatattttgt tattggcctt ttctattcct gtaatgaaag ctgtttgtcg taacttgaaa |
| 5821 | ttttatcttt tactatggga gtcactattt attattgctt atgtgccctg ttcaaaacag |
| 5881 | aggcacttaa tttgatcttt tatttttctt tgtttttatt ttttttttta tttagatgac |
| 5941 | caaaggtcat tacaacctgg ctttttattg tatttgtttc tggtctttgt taagttctat |
| 6001 | tggaaaaacc actgtctgtg tttttttggc agttgtctgc attaacctgt tcatacaccc |
| 6061 | attttgtccc tttattgaaa aaataaaaaa aattaaagta ca |

SEQ ID NO: 11 is an amino acid sequence of Isoform 1 of human BCL11A.

| | |
|---|---|
| 1 | msrrkqgkpq hlskrefspe pleailtdde pdhgplgape gdhdlltcgq cqmnfplgdi |
| 61 | lifiehkrkq cngslcleka vdkppspspi emkkasnpve vgiqvtpedd dclstssrgi |
| 121 | cpkgehiadk llhwrglssp rsahgalipt pgmsaeyapq gickdepssy tcttckqpft |
| 181 | sawfllqhaq nthglriyle sehgspltpr vgipsglgae cpsqpplhgi hiadnnpfnl |
| 241 | lripgsvsre asglaegrfp ptpplfsppp rhhldphrie rlgaeemala thhpsafdrv |
| 301 | lrlnpmamep pamdfsrrlr elagntsspp lspgrpspmq rllqpfqpgs kppflatppl |
| 361 | pplqsappps qppvksksce fcgktfkfqs nlvvhrrsht gekpykcnlc dhactqaskl |
| 421 | krhmkthmhk sspmtvksdd glstasspep gtsdlvgsas salksvvakf ksendpnlip |
| 481 | engdeeeeed deeeeeeeee eeeelteser vdygfglsle aarhhenssr gavvgvgdes |
| 541 | ralpdvmqgm vlssmqhfse afhqvlgekh krghlaeaeg hrdtcdedsv agesdriddg |
| 601 | tvngrgcspg esasgglskk lllgspssls pfskriklek efdlppaamp ntenvysqwl |
| 661 | agyaasrqlk dpflsfgdsr qspfassseh ssengslrfs tppgeldggi sgrsgtgsgg |
| 721 | stphisgpgp grpsskegrr sdtceycgkv fkncsnltvh rrshtgerpy kcelcnyaca |
| 781 | qsskltrhmk thgqvgkdvy kceickmpfs vystlekhmk kwhsdrvlnn dikte |

Alternative splicing of BCL 11a leads to four isoforms containing 1, 3, or 6 C2H2 zinc-finger domains required for DNA-binding. Isoform 1, also known as BCL11A-XL, contains 6 C2H2 zinc-finger domains, and is the most abundant isoform in erythroid cells. Zinc finger domains are located at the following amino acid sequences:

Zinc finger domain 1 amino acids: 170-193
(SEQ ID NO: 67: ytcttckqpftsawfllqhaqnth);
Zinc finger domain 2 amino acids: 377-399
(SEQ ID NO: 68: kscefcgktfkfqsnlvvhrrsh);
Zinc finger domain 3 amino acids: 405-429
(SEQ ID NO: 69: ykcnlcdhactqasklkrhmkthmh);
Zinc finger domain 4 amino acids: 742-764
(SEQ ID NO: 70: dtceycgkvfkncsnltvhrrsh);
Zinc finger domain 5 amino acids: 770-792
(SEQ ID NO: 71: ykcelcnyacaqsskltrhmkth);
Zinc finger domain 6 amino acids: 800-823
(SEQ ID NO: 72: ykceickmpfsvystlekhmkkwh).

BCL11A can be targeted for degradation in erythroid cells, for example, through introduction of a bispecific construct as described herein that includes an antibody or antigen-binding fragment thereof that specifically binds BCL11A and a binding element that specifically binds TRIM10 or TRIM58. In one embodiment, the binding element that specifically binds TRIM10 or TRIM58 can be an antibody or antigen-binding fragment thereof. Any of a number of ways to introduce the construct to erythroid cells can be used, including, but not limited to introduction of the construct or a nucleic acid encoding it via lipid nanoparticles, introduction (e.g., as a fusion) via cell-penetrating peptides, or introduction of a vector, e.g., a viral vector, among others, encoding the construct.

BCL11A can also be targeted for degradation, for example, through introduction of a fusion construct as described herein that includes an antibody or antigen-binding fragment thereof that specifically binds BCL11A fused to a TRIM10 or TRIM58 polypeptide. The fusion construct can be introduced to erythroid cells in any of a number of ways, including, but not limited to introduction of the construct or a nucleic acid encoding it via lipid nanoparticles, introduction (e.g., as a fusion) via cell-penetrating peptides, or introduction of a vector, e.g., a viral vector, among others, encoding the construct.

### Antibodies

As discussed above, in various embodiments, described herein are antibodies or antigen-binding fragments thereof that specifically bind to BCL11A or to a TRIM10 or TRIM58 polypeptide. There are a wide variety of antibodies and constructs based upon them, but each generally includes an antigen-binding structure that includes so-called complementarity determining regions (CDRs) of an immunoglobulin polypeptide separated by so-called “framework regions” of the immunoglobulin. The CDRs are highly variable between antibodies that bind different antigens, while the framework regions tend to be more conserved. Most naturally-occurring antibodies include six CDRs, with three contributed by a so-called heavy chain variable domain, V_{H}, and three contributed by a so-called light chain variable domain, V_{L}. In these antibodies, the V_{H} and V_{L} domains form a complex in which residues in the CDRs of each chain are configured to make contact with an epitope on a given antigen, thereby conferring binding specificity for that epitope of that antigen. While the majority of naturally-occurring antibodies include the V_{H}/V_{L}, six CDR configuration, several classes of animals, including camelids and cartilaginous fishes, produce antibodies that include only a V_{H} domain, with three CDRs. As discussed further below, the discovery of such antibodies, and the recognition that they can bind to target antigens with specificity and avidity closely comparable to that of antibodies with six CDRs has spawned a movement to isolate so-called single domain antibodies that are highly specific for target antigens. The movement has developed approaches for the generation or selection of single domain antibodies based on immunization of camelid species, selection from libraries generated from pre-immune and immune camelid genes, humanized camelid-based antibodies, as well as approaches for the selection of human single domain antibodies from libraries of pre-immune or immune V region human genes. Among the benefits of single domain antibodies are their smaller size, which makes it easier to introduce them to cells, better suited to the packaging constraints of, e.g., viral vectors and better suited as fusion partners in bi- or multifunctional constructs. The following provides additional detail in regard to antibodies generally and single domain antibodies more specifically.

As used herein, the term “antibody” refers to IgG, IgM, IgA, IgD or IgE molecules or antigen-binding fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule. An epitope is that portion of an antigen molecule with which an antibody makes direct physical contact via its antigen-binding site when the antibody specifically binds the antigen.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions, as occurs, for example, in an IgG immunoglobulin. The term “antibody reagent” encompasses antigen-binding fragments of antibodies (e.g., single chain antibodies, Fab and sFab fragments, F(ab′)2, Fd fragments, Fv fragments, scFv, and single domain antibodies (sdAb) fragments (see, e.g. de Wildt et al., Eur J. Immunol. 1996; 26 (3): 629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

As noted above, the V_{H} and V_{L} regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). The term “complementarity determining region” or “CDR” refers to variable regions in antibody polypeptides and contains the amino acid sequences that mediate specific binding to antigenic targets. These CDR regions account for the basic specificity of the antibody or antigen-binding fragment thereof for a particular antigenic determinant structure. Such regions are also referred to as “hypervariable regions.” Each V_{H} and V_{L} is typically composed of three CDRs and four FRs, arranged from amino-terminus to carboxy-terminus in the following order: FR1, CDR1, FR2, CDR2, FR3, CDR3, FR4. Single domain antibodies have three CDR regions, each non-contiguous with the others (termed CDR1, CDR2, CDR3) and separated by framework regions.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the V_{L}, V_{H}, CL and CHI domains; (ii) a F(ab′)2 fragment, a bivalent fragment including two Fab fragments linked by a disulfide bridge at the hinge region; (iii) an Fd fragment consisting of the V_{H} and CHI domains; (iv) an Fv fragment consisting of the V_{L} and V_{H} domains of a single arm of an antibody, (v) a dAb fragment (Ward et al., (1989) Nature 341:544-546; which is incorporated by reference herein in its entirety), which consists of a V_{H} or V_{L} domain; and (vi) an isolated complementarity determining region (CDR) that retains specific antigen-binding functionality.

In some embodiments, a target ligand-binding recognition domain is a single-domain antibody. By the term “single-domain antibody” or “sdAb”, it is meant an antibody fragment comprising a single protein domain that specifically binds a target antigen. A single domain antibody requires only three CDRs to specifically bind its target antigen. Single domain antibodies can comprise any variable fragment, including V_{L}, V_{H}, V_{HH} (camelid), and V_{NAR} (shark), and can be naturally-occurring or produced by recombinant technologies. For example, V_{H}, V_{L}, V_{HH}, and V_{NAR} domains can be generated by techniques well known in the art (Holt, et al., 2003; Jespers, et al., 2004a; Jespers, et al., 2004b; Tanha, et al., 2001; Tanha, et al., 2002; Tanha, et al., 2006; Revets, et al., 2005; Holliger, et al., 2005; Harmsen, et al., 2007; Liu, et al., 2007; Dooley, et al., 2003; Nuttall, et al., 2001; Nuttall, et al., 2000; Hoogenboom, 2005; Arbabi-Ghahroudi et al., 2008). In the recombinant DNA technology approach, libraries of sdAbs can be constructed in a variety of ways, “displayed” in a variety of formats such as phage display, yeast display, ribosome display, and subjected to selection to isolate binders to the targets of interest (panning). Examples of libraries include immune libraries derived from llama, shark or human immunized with the target antigen; non-immune/naïve libraries derived from non-immunized llama, camel, shark or human; or synthetic or semi-synthetic libraries such as V_{H}, V_{L}, V_{HH} Or V_{NAR} libraries. In one embodiment, the sdAb can be a heavy variable domain (V_{H}). The term includes single domain antibodies as initially identified by selection/isolation of clones, as well as affinity-matured versions prepared by mutagenesis of isolated candidates.

In some embodiments, the target ligand-binding recognition domain is a nanobody. A “nanobody” (Nb) is a single variable domain (V_{H}H) single domain antibody generally derived, whether via immunization or via recombinant techniques, from for example, camelids, alpacas, llamas, and sharks. Nanobodies generally comprise a single amino acid chain that can be considered to comprise four framework regions and three complementarity determining regions. The term “camelids” refers to old world camelids (Camelus bactrianus and Camelus dromedarius) and new world camelids (for example, Lama paccos, Lama glama, Lama guanicoe and Lama vicugna). The small size and unique biophysical properties of Nbs exceed conventional antibody fragments for the recognition of uncommon or hidden epitopes and for binding into cavities or active sites of protein targets. Further, Nbs can be designed as multi-specific and multivalent antibodies or attached to reporter molecules. Certain Nbs and Nb variants can survive the gastro-intestinal system and Nbs can easily be manufactured. Therefore, Nbs can be used in many applications including drug discovery and therapy, but also as a versatile and valuable tool for purification, functional study and crystallization of proteins.

As used herein, the term “specific binding” refers to a physical interaction between two molecules, compounds, cells and/or particles wherein the first entity binds to the second, target entity with greater specificity and affinity than it binds to a third entity which is a non-target. A reagent specific for a given target is one that exhibits specific binding for that target under the conditions of the assay being utilized. The specificity of an antibody or antibody fragment thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as an antibody or antigen-binding fragment thereof: the less the value of the K_{D}, the stronger the binding strength between an antigenic determinant and the antigen-binding molecule. Alternatively, the affinity can also be expressed as the affinity constant (KA), which is 1/K_{D}). Accordingly, an antibody or antigen-binding fragment thereof as described herein is said to be “specific for” or to “specifically bind” or “selectively bind” a first target or antigen compared to a second target or antigen when it binds to the first antigen with an affinity (as described above, and suitably expressed, for example as a K_{D} value) that is at least 1000 times, 10000 times or more better than the affinity with which said amino acid sequence or polypeptide binds to another given polypeptide. Generally, a molecule that “specifically binds,” “selectively binds” or “is specific for” a given target will bind with a K_{D} of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which polypeptide agents as described herein selectively bind the target using any suitable methods, such as titration of a polypeptide agent in a suitable cell binding assay.

It should be understood in this context that the specific binding is mediated by the CDRs of the antibody polypeptide, as opposed to any other portion of the antibody polypeptide.

Antibody dissociation constants and affinities can be determined, for example, by a surface plasmon resonance based assay (such as the BIACORE assay described in PCT Application Publication No. WO2005/012359); Forte Bio Octet™ analysis, enzyme-linked immunosorbent assay (ELISA); and competition assays (e.g., RIA's), for example.

As used herein, “avidity” is a measure of the strength of binding between an antigen-binding molecule (such as an antibody or antibody fragment thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D}) of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (KA) of 105 to 1012 liter/moles or more, such as 107 to 1012 liter/moles or 108 to 1012 liter/moles). Any K_{D} value greater than 10⁻⁴ mol/liter (or any KA value lower than 104 M-1) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner, including, for example, Scatchard analysis and/or competitive binding assays, such as radioimmunoassays (RIA), enzyme immunoassays (EIA) and sandwich competition assays, and the different variants thereof known in the art; as well as other techniques as known in the art and/or mentioned herein. The person of ordinary skill in the art can determine appropriate conditions under which polypeptide agents as described herein selectively bind the targets using any suitable methods, such as titration of a polypeptide agent in a suitable binding assay.

As used herein, the term “selectively inhibits” means that an agent inhibits, as that term is used herein, the function or activity of a given target but does not substantially inhibit the function or activity of a relevant non-target. Thus, for example, an antibody polypeptide that selectively inhibits the binding or function of BCL11A will not substantially inhibit the binding or function of the structurally-related BCL11B polypeptide.

As used herein, the term “target” refers to a biological molecule (e.g., peptide, polypeptide, protein, nucleic acid, lipid, carbohydrate, etc.) to which a polypeptide domain which has a binding site can selectively bind. The target can be, for example, an intracellular target (e.g., an intracellular protein target) or a cell surface target (e.g., a membrane protein, a receptor protein). Exemplary “target” biological molecules for the purposes of the methods and compositions described herein include BCL11A. In some embodiments, a “target” can be one which it is wished to specifically degrade or inhibit, which can be the case when BCL11A is a target. In other instances, a “target” can be one which it is wished to bind and tether to another moiety, which can be the case when the target is, e.g., a TRIM10 or TRIM58 polypeptide.

As used herein, an antibody reagent (e.g., an antibody or antigen-binding domain thereof) that specifically binds to BCL11A, binds BCL11A with a dissociation constant (K_{D}) of 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less and binds to that target at least 100×, 1000×, or 10,000× more strongly than it binds to an off-target protein or distinct cell-surface or intracellular marker. An antibody reagent that specifically binds BCL11A will bind BCL11B, if at all, with a K_{D} at least 100×, or at least 1000× greater than the K_{D} with which it binds to BCL11A.

Similarly, an antibody reagent that specifically binds to TRIM10 or to TRIM58 binds to TRIM10 or TRIM58 with a dissociation constant (K_{D}) of 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less and binds to that target at least 100×, 1000×, or 10,000× more strongly than it binds to an off-target protein or distinct cell-surface or intracellular marker.

Additionally, and as described herein, a recombinant antibody or antigen-binding domain thereof can be further optimized to decrease potential immunogenicity, while maintaining functional activity, e.g., for therapy in humans. In this regard, functional activity means a polypeptide capable of displaying one or more known functional activities associated with an antibody or antibody reagent thereof as described herein. Such functional activities include, e.g. the ability to specifically bind to a target. One approach for decreasing potential immunogenicity of an antibody or antigen-binding fragment thereof is referred to as “humanizing” the antibody or antigen-binding fragment thereof. The term “humanized antibody” refers to forms of antibodies (or an antigen-binding fragment thereof) that contain sequences from non-human (e.g., murine) antibodies as well as human antibodies. Such antibodies are chimeric antibodies which contain minimal sequence derived from non-human immunoglobulin. In general, a humanized antibody or antigen-binding fragment will ideally comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). The constant region, can if desired, include one or more modifications that modify or disrupt interaction of the human or humanized antibody with an Fc receptor. Humanization can essentially be performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent, camelid or shark framework sequences with the corresponding sequences of a human antibody.

As discussed above, antibody polypeptides are provided herein that specifically bind to BCL11A and do not substantially cross-react with BCL11B. Non-limiting examples include the following nanobodies that target different zinc finger domains of the BCL11A polypeptide.

Nanobodies that Target BCL11A ZNF6

The following provides amino acid sequence for nanobodies that specifically bind human BCL11A.

NB14
SEQ ID NO: 12
QVQLVESGGG LVQAGGSLRL SCAASGSIFS FYAMGWYRQA PGKEREFVAS ITWRDDSTYY (60)
ADSVKGRFTI SRDNAKNTVY LQMNSLKPED TAVYYCAADT DDYTVEMDYW GQGTQVTVSS (120)
NB15
SEQ ID NO: 13
QVQLVESGGG LVQAGGSLRL SCAASGSTFS DYAMGWYRQA PGKERELVAV ITASDDITYY (60)
ADSVKGRFTI SRDNAKNTVY LQMNSLKPED TAVYYCAAAV TPG-QDLDYW GQGTQVTVSS (119)
NB53
SEQ ID NO: 14
QVQLVESGGG LVQAGGSLRL SCAASGSTFS GYAMGWYRQA PGKERELVAA ITSSGASTYY (60)
ADSVKGRFTI SRDNAKNTVY LQMNSLKPED TAVYYCAALD EGY-LDYDSW GQGTQVTVSS (119)
NB61
SEQ ID NO: 15
QVQLVESGGG LVQAGGSLRL SCAASGFIFD SYAMGWYRQA PGKERELVAA ITSSGSSTYY (60)
ADSVKGRFTI SRDNAKNTVY LQMNSLKPED TAVYYCAALD --Y-VIDGYW GQGTQVTVSS (117)
NB19 (Nb6101-M45D-19)
SEQ ID NO: 16
RVQLVESGGG LVQAGGSLRL SCAADGFDFK SYAMGWYRQA PGREDELVAA ITASGDYTYY (60)
ADSVKGRFTI SRDNAKNTVY LQMNSLKPDD TAVYYCAALD --Y-VAEGYW GQGTQVTVSS (117)
NB5344-N74D
SEQ ID NO: 17
QVQLVKSGGG LVQAGDSLRL SCAASGSTFS GYAMGWYRQA PGKERELVAA ITSSGASTYY (60)
ADSVRGRFTI SRDNAKNTVY LQMNSLKPED TAVYYCAALD EGY-LDYDSW GQGTQVTVSS (119)
NB6101
SEQ ID NO: 18
RVQLVESGGG LVQAGGSLRL SCAASGFIFD SYAMGWYRQA PGKEMELVAA ITSSGSSTYY (60)
ADSVKGRFTI SRDNAKNTVY LQMNSLKPED TAVYYCAALD --Y-VIDGYW GQGTQVTVSS (117)
NB6101-M45D
SEQ ID NO: 19
RVQLVESGGG LVQAGGSLRL SCAASGFIFD SYAMGWYRQA PGKEDELVAA ITSSGSSTYY (60)
ADSVKGRFTI SRDNAKNTVY LQMNSLKPED TAVYYCAALD --Y-VIDGYW GQGTQVTVSS (117)
NB1453
SEQ ID NO: 20
QVQLVESGGG LVQAGGSLHL SCAVSGSIFS FYAMGWYRQA PGKEREFVAS ITRRDDSTYY (60)
ADSVEGRFAI SRDNAKNTVY LQMNSLKPED TAVYYCAADT DDYTVEMDYW GQGTQVTVSS (120)
NB1459
SEQ ID NO: 21
QVQLVESGGG LVQAEGSLRL SCAASGSIFS FYAMGWYRQA PGKEREFVAS ITWRDDSTYY (60)
ADSVTGRFTI SRDNAKNTVY LQMNSLKPED TAVYYCAADT DDYSVEMDYW GQGTQVTVSS (120)
NB1431
SEQ ID NO: 22
QVQLVESGGG LVQAGGCLRL SCAASGSIFS FYAMGWYRQA PGNEREFVAS ITWRDDSTYY (60)
ADSVKGRFTI SRDNAKNTVY LQMNSLKPED TAVYYCAADT DDYTVEMDYW GQGTHVTVSS (120)
NB1407
SEQ ID NO: 23
QVQLVESGGD LVQAGASLRL SCAASGSIFS FYAMGWYRQA PGKEREFVAS ITWRDDSTYY (60)
ADSVKGRFTI SRDNARNTVY LQMNNLKPED TAVYYCAADT DDYTVEMDYW GQGTQVTVSS (120)
NB1481
SEQ ID NO: 24
QVQLVESGGG LVQAGGSLRL SCAASGSIFS FYAMGWYRQA PGKEREFVAF ITWRDDSTYY (60)
ADSVKGRFRI SRDNAKNTVY LQMNSLKPED TAVYYCAADT DDYTVEMDYW GQGTQVTVSS (120)
NB1419
SEQ ID NO: 25
QVQLVESGGD LVQAGGSLRL SCAASGSIFS FYAMGWYRQA PGKEREFVAS ITWRDDSTYY (60)
ADSVKGRFTI SRDNAKNTVY LOMNRLKPED TAVYYCAADT DDYTVEMDYW GQGTQVTVSS (120)
NB1487
SEQ ID NO: 26
QVQLVESGGG LVQAGGSLRL SCAASGSIFS FYAMGWYRQA PGKEREFVAS ITWRDDSTYY (60)
ADSVMGRFTI SRDNAKNTVY LQMNSLKPED TAVYYCAADT DDYTVEMDYW GQGTQVTVSS (120)
NB6101-M45D-14
SEQ ID NO: 27
QVQLVESGGG LVQAGDSLRL SCAASGSTFS GYAMGWYRQA PGKERELVAA ITSSGASTYY (60)
ADSVRGRFTI SRDNAKNTVY LQMNSLKPED TAVYYCAALD EGY-LDYDSW GQGTQVTVSS (119)
NB6130
SEQ ID NO: 28
QVQFVESGGG FVQAGGSLRL SCAASGFIFD SYAMGWYSQA PGKERELVAA ITSSGSSTYY (60)
ADSVKGRFTI SRDNAKNTVY LQMNSLKPED TAVYYCAALD --Y-VIDGYW GQGTQVTVSS (117)
NB6127
SEQ ID NO: 29
QVQLVESGGG LVQAGGSLRL SCAASGFIFD SYSMGWYRQA PGKEGELVAA ITSSGSSTYY (60)
ADSVNGRFTI SRDNAKNTVY LQMNSLKPED TAVYYCAALD --Y-VIDGYW GQGTQVTVSS (117)
NB6160
SEQ ID NO: 30
QVQLVESGGG LVQAGGSLRL SCAASGFIFD SYAMGWYRQA PGKEGELVAA ITSSGSSTYY (60)
ADSVKGRFTI SRDNAKNTVY LQMNSLKPED TAVYYCAALD --Y-VIDGYW GQGTQVSVSS (117)
NB6102
SEQ ID NO: 31
QVQLVESGGG LVQAGGSLRL SCAASGFIFD SYAMGWYRQA PGKEGELVAA ITSSGSSTYY (60)
ADSVKGRFTI SRDNAKNTVY LQMNSLKPED TAVYYCAALD --Y-VIDGYW GQGTQVTVSS (117)
NB6101-M45D-26
SEQ ID NO: 32
RVQLVESGGG LVQAGGSLRL SCAADGFDFS SYAMGWYRQA PGTQDELVAA ITASGSYTYY (60)
SDSVKGRFTI SRDNAKNTVY LQMNSLKPDD TAVYYCAALS --Y-VAEGYW GQGTQVTVSS (117)
NB6101-M45D-20
SEQ ID NO: 33
RVQLVESGGG LVQAGGSLRL SCAADGFDFK SYAMGWYRQA PGYEDELVAA ITASGSYTYY (60)
SDSVKGRFTI SRDNAKNTVY LQMNSLKPED TAVYYCAALS --Y-VAEGYW GQGTQVTVSS (117)

TABLE 1
CDR's for Nanobodies directed to ZNF6

| Nanobody | CDR1 | CDR2 | CDR3 |
|---|---|---|---|
| Nb14 | GSIFS FYAM \|\| SEQ ID NO: 43 | EFVAS ITWRDDSTYY \|\| SEQ ID NO: | ADT DDYTVEMDYW \|\| |
| | | 51 | SEQ ID NO: 60 |
| Nb15 | GSTFS DYAM \|\| SEQ ID NO: | ELVAV ITASDDITYY \|\| SEQ ID NO: 52 | AAV TPG-QDLDYW \|\| |
| | 44 | | SEQ ID NO: 61 |
| Nb53 | GSTFS GYAM \|\| SEQ ID NO: | ELVAA ITSSGASTYY \|\| SEQ ID NO: 53 | ALD EGY-LDYDSW \|\| |
| | 45 | | SEQ ID NO: 62 |
| Nb61 | GFIFD SYAM \|\| SEQ ID NO: 46 | ELVAA ITSSGSSTYY \|\| SEQ ID NO: 54 | ALD --Y-VIDGYW \|\| |
| | | | SEQ ID NO: 63 |
| Nb19 | GFDFK SYAM \|\| SEQ ID NO: | ELVAA ITASGDYTYY \|\| SEQ ID NO: | ALD --Y-VAEGYW \|\| |
| (Nb6101- | 47 | 55 | SEQ ID NO: 64 |
| M45D-19) | | | |
| Nb5344- | GSTFS GYAM \|\| SEQ ID NO: | ELVAA ITSSGASTYY \|\| SEQ ID NO: 53 | ALD EGY-LDYDSW \|\| |
| N74D | 45 | | SEQ ID NO: 62 |
| Nb6101 | GFIFD SYAM \|\| SEQ ID NO: 46 | ELVAA ITSSGSSTYY \|\| SEQ ID NO: 54 | ALD --Y-VIDGYW \|\| |
| | | | SEQ ID NO: 63 |
| Nb6101- | GFIFD SYAM \|\| SEQ ID NO: 46 | ELVAA ITSSGSSTYY \|\| SEQ ID NO: 54 | ALD --Y-VIDGYW \|\| |
| M45D | | | SEQ ID NO: 63 |
| Nb1453 | GSIFS FYAM \|\| SEQ ID NO: 43 | EFVAS ITRRDDSTYY \|\| SEQ ID NO: | ADT DDYTVEMDYW \|\| |
| | | 56 | SEQ ID NO: 60 |
| Nb1459 | GSIFS FYAM \|\| SEQ ID NO: 43 | EFVAS ITWRDDSTYY \|\| SEQ ID NO: | ADT DDYSVEMDYW \|\| |
| | | 57 | SEQ ID NO: 65 |
| Nb1431 | GSIFS FYAM \|\| SEQ ID NO: 43 | EFVAS ITWRDDSTYY \|\| SEQ ID NO: | ADT DDYTVEMDYW \|\| |
| | | 57 | SEQ ID NO: 60 |
| Nb1407 | GSIFS FYAM \|\| SEQ ID NO: 43 | EFVAS ITWRDDSTYY \|\| SEQ ID NO: | ADT DDYTVEMDYW \|\| |
| | | 57 | SEQ ID NO: 60 |
| Nb1481 | GSIFS FYAM \|\| SEQ ID NO: 43 | EFVAF ITWRDDSTYY \|\| SEQ ID NO: | ADT DDYTVEMDYW \|\| |
| | | 58 | SEQ ID NO: 60 |
| Nb1419 | GSIFS FYAM \|\| SEQ ID NO: 43 | EFVAS ITWRDDSTYY \|\| SEQ ID NO: | ADT DDYTVEMDYW \|\| |
| | | 57 | SEQ ID NO: 60 |
| Nb1487 | GSIFS FYAM \|\| SEQ ID NO: 43 | EFVAS ITWRDDSTYY \|\| SEQ ID NO: | ADT DDYTVEMDYW \|\| |
| | | 57 | SEQ ID NO: 60 |
| Nb6101- | GSTFS GYAM \|\| SEQ ID NO: 45 | ELVAA ITSSGASTYY \|\| SEQ ID NO: 53 | ALD EGY-LDYDSW \|\| |
| M45D-14 | | | SEQ ID NO: 62 |
| Nb6130 | GFIFD SYAM \|\| SEQ ID NO: 46 | ELVAA ITSSGSSTYY \|\| SEQ ID NO: 54 | ALD --Y-VIDGYW \|\| |
| | | | SEQ ID NO: 63 |
| Nb6127 | GFIFD SYSM \|\| SEQ ID NO: 48 | ELVAA ITSSGSSTYY \|\| SEQ ID NO: 54 | ALD --Y-VIDGYW \|\| |
| | | | SEQ ID NO: 63 |
| Nb6160 | GFIFD SYAM \|\| SEQ ID NO: 46 | ELVAA ITSSGSSTYY \|\| SEQ ID NO: 54 | ALD --Y-VIDGYW \|\| |
| | | | SEQ ID NO: 63 |
| Nb6102 | GFIFD SYAM \|\| SEQ ID NO: 46 | ELVAA ITSSGSSTYY \|\| SEQ ID NO: 54 | ALD --Y-VIDGYW \|\| |
| | | | SEQ ID NO: 63 |
| Nb6101- | GFDFS SYAM \|\| SEQ ID NO: | ELVAA ITASGSYTYY \|\| SEQ ID NO: 59 | ALS --Y-VAEGYW \|\| |
| M45D-26 | 49 | | SEQ ID NO: 64 |
| NB6101- | GFDFK SYAM \|\| SEQ ID NO: | ELVAA ITASGSYTYY \|\| SEQ ID NO: 59 | ALS --Y-VAEGYW \|\| |
| M45D-20 | 50 | | SEQ ID NO: 64 |

### Nanobodies Directed to ZNF23 “Extended Region”

2D9/Consensus
SEQ ID NO: 34
QVQLVESGGG LVQAGGSLRL SCAASGSIFV NNAMGWYRQA PGKERELVAA ISASGGSTYY (60)
ADSVKGRFTI SRDNAKNTVY LQMNSLKPED TAVYYCAADQ DVYPYEYWGQ GTQVTVSS (118)
V102G
SEQ ID NO: 35
QVQLVESGGG LVQAGGSLRL SCAASGSIFV NNAMGWYRQA PGKERELVAA ISASGGSTYY (60)
ADSVKGRFTI SRDNAKNTVY LQMNSLKPED TAVYYCAADQ DGYPYEYWGQ GTQVTVSS (118)
W108L
SEQ ID NO: 36
QVQLVESGGG LVQAGGSLRL SCAASGSIFV NNAMGWYRQA PGKERELVAA ISASGGSTYY (60)
ADSVKGRFTI SRDNAKNTVY LQMNSLKPED TAVYYCAADQ DVYPYEYLGQ GTQVTVSS (118)

### CDR's for Nanobodies Directed to ZNF23 Extended Region

2D9/V102G/W108L CDR 1
SEQ ID NO: 37
SIFVNNAM
2D9/V102G/W108L CDR 2
SEQ ID NO: 38
ELVAAISASGGSTYY
2D9 CDR 3
SEQ ID NO: 39
ADQDVYPYEYW
2D9 CDR 3
SEQ ID NO: 40
ADQDGYPYEYW
2D9 CDR 3
SEQ ID NO: 41
ADQDVYPYEYL

### Nanobodies Directed to BCL11A ZNF4

Nb12
SEQ ID NO: 42
QVQLVESGGG SVQPGGSLTL SCTASGFPLS MWGMTWLRQA PGKGLERVSG IASDSTNTYY (60)
ADSVKGRFAI SRDNAKNKLF LQMNSLKPED TAVYFCARKW TNWDLKGQGT QVTVSS (116)

As a means of describing structure involved in or necessary for binding to BCL11A, the nanobody NB14 of SEQ ID NO: 12 is used as a reference herein. It should be understood that other nanobodies described herein can be used as a reference in a similar manner. The following Table 2 shows the amino acid sequence of NB14, with variations at each site that can permit specific binding of the nanobody to the same epitope of BCL11A. In some embodiments, variation relative to SEQ ID NO: 12 occurs only at the sites listed in Table 2.

TABLE 2
BCL11A ZNF6 nanobody amino acid variations.
Compared to Nb14 (SEQ ID NO: 12).

| Amino | Nb14 | Potential |
|---|---|---|
| Acid | amino | site specific |
| # | acid | mutations |
| 1 | Q | R; |
| 2 | V | M |
| 4 | L | F |
| 6 | E | K |
| 10 | G | D |
| 11 | L | F |
| 15 | G | E; D |
| 16 | G | A; D |
| 17 | S | C |
| 19 | R | H |
| 24 | A | V |
| 25 | S | D |
| 27 | S | F; Y |
| 28 | I | T; D |
| 30 | S | D; K |
| 31 | F | D; G; S |
| 33 | A | S |
| 38 | R | S |
| 43 | K | N; T; Y; R |
| 44 | E | Q |
| 45 | R | G; M; D; |
| 46 | E | Q |
| 47 | F | L |
| 50 | S | F; V; A |
| 53 | W | R; A; S |
| 54 | R | S |
| 55 | D | G |
| 56 | D | A; S |
| 57 | S | I; Y; E |
| 61 | A | S; H |
| 62 | D | P |
| 65 | K | E; T; M; R; N |
| 69 | T | A; R |
| 76 | K | R |
| 85 | S | N; R |
| 89 | E | D |
| 99 | D | A; L |
| 100 | T | V; D; S |
| 101 | D | E; S; T; V; — |
| 102 | D | V; P; G; — |
| 103 | Y | A; S; K; G |
| 104 | T | E; Q; S; — |
| 105 | V | G; D; Q; L |
| 106 | E | Y; D; I; A |
| 107 | M | W; L; Y; D; E |
| 108 | D | S; G |
| 109 | Y | Q; W; S |
| 111 | G | T |
| 112 | Q | G |
| 113 | G | T; V |
| 114 | T | Q |
| 115 | Q | H; V |
| 116 | V | S; T |
| 117 | T | S; V |

### Hemoglobinopathies

Fetal hemoglobin (HbF) is a tetramer of two adult α-globin polypeptides and two fetal β-like γ-globin polypeptides. During gestation, the duplicated γ-globin genes constitute the predominant genes transcribed from the β-globin locus. Following birth, γ-globin becomes progressively replaced by adult β-globin, a process referred to as the “fetal switch.” In humans, the developmental switch from production of predominantly fetal hemoglobin or HbF (α2γ2) to production of adult hemoglobin or HbA (α2β2) begins at about 28 to 34 weeks of gestation and continues shortly after birth at which point HbA becomes predominant. This switch results primarily from decreased transcription of the gamma-globin genes and increased transcription of beta-globin genes. On average, the blood of a normal adult contains only about 2% HbF, though residual HbF levels have a variance of over 20 fold in healthy adults (Atweh, Semin. Hematol. 38 (4): 367-73 (2001)).

Hemoglobinopathies encompass a number of anemias of genetic origin in which there are insufficient amounts of hemoglobin capable of carrying oxygen in red blood cells (RBCs). These disorders include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. Those disorders specifically associated with the β-globin protein are referred to generally as β-hemoglobinopathies. For example, β-thalassemias result from a partial or complete defect in the expression of the β-globin gene, leading to deficient or absent HbA. Sickle cell anemia or sickle cell disease (SCD) results from a point mutation in the β-globin structural gene, leading to the production of an abnormal hemoglobin (HbS) that results in deformed (sickled) RBCs. HbS RBCs are more fragile than normal RBCs and undergo hemolysis more readily, leading eventually to anemia (Atweh, Semin. Hematol. 38 (4): 367-73 (2001)).

The search for treatment aimed at reduction of globin chain imbalance in patients with β-hemoglobinopathies has focused on the manipulation of fetal hemoglobin (α2γ2; HbF). The important therapeutic potential of such approaches is indicated by observations of the mild phenotype of individuals with co-inheritance of both homozygous β-thalassemia and hereditary persistence of fetal hemoglobin (HPFH), as well as by those patients with homozygous β-thalassemia who synthesize no adult hemoglobin, but in whom a reduced requirement for transfusions is observed in the presence of increased concentrations of fetal hemoglobin. Furthermore, it has been observed that certain populations of adult patients with β chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease (Pembrey, et al., Br. J. Haematol. 40:415-429 (1978)). Thus, it is now accepted that β-hemoglobinopathies, such as sickle cell anemia and the β-thalassemias, can be ameliorated by increased HbF production. (Reviewed in Jane and Cunningham Br. J. Haematol. 102:415-422 (1998) and Bunn, N. Engl. J. Med. 328:129-131 (1993)).

As used herein, treating or reducing a risk of developing a hemoglobinopathy in a subject means to ameliorate at least one symptom of hemoglobinopathy. In one aspect, the methods described herein feature methods of treating, e.g., reducing severity or progression of, a hemoglobinopathy in a subject. In another aspect, the methods can also be used to reduce a risk of developing a hemoglobinopathy in a subject, delaying the onset of symptoms of a hemoglobinopathy in a subject, or increasing the longevity of a subject having a hemoglobinopathy. In one aspect, the methods can include selecting a subject on the basis that they have, or are at risk of developing a hemoglobinopathy, but do not yet have symptoms of a hemoglobinopathy. Selection of a subject can include detecting symptoms of a hemoglobinopathy, a blood test, genetic testing, or clinical recordings. If the results of the test(s) indicate that the subject has a hemoglobinopathy, the methods can also include administering a composition as described herein, thereby treating, or reducing the risk of developing, a hemoglobinopathy in the subject. As non-limiting examples, a subject with a diagnosis of SCD with genotype HbSS, HbS/BO thalassemia, HbSD, or HbSO, and/or HbF<10% by electrophoresis is indicated for treatment using compositions and methods as described herein. By the phrase “risk of developing disease” is meant the relative probability that a subject will develop a hemoglobinopathy in the future as compared to a control subject or population (e.g., a healthy subject or population). For example, an individual carrying the genetic mutation associated with SCD, an A to T mutation of the β-globin gene, and whether the individual in heterozygous or homozygous for that mutation increases that individual's risk. Methods that promote the de-repression or re-expression of fetal hemoglobin in an individual diagnosed with or suffering from a hemoglobinopathy can be effective for treatment of the disease or disorder.

As used herein, the term “hemoglobinopathy” refers to a condition involving the presence of an abnormal hemoglobin molecule or insufficient levels of hemoglobin capable of carrying oxygen in the blood and releasing the oxygen in tissues of the body. The term refers to a condition involving any defect in the structure, function or amount of any hemoglobin of an individual, and includes defects in the primary, secondary, tertiary or quaternary structure of hemoglobin caused by any mutation, such as deletion mutations or substitution mutations in the coding regions of a globin gene, or mutations in, or deletions of, the promoters or enhancers of such genes that cause a reduction in the amount of hemoglobin produced as compared to a normal or standard condition. The term further includes any decrease in the amount or effectiveness of hemoglobin, whether normal or abnormal, caused by external factors such as disease, chemotherapy, toxins, poisons, or the like.

The term “sickle cell disease” or “SCD” is defined herein to include any symptomatic anemic condition which results from sickling of red blood cells. Manifestations of SCD include: anemia; pain; and/or organ dysfunction, such as renal failure, retinopathy, acute-chest syndrome, ischemia, priapism, and stroke. The term refers to a variety of clinical problems attendant upon SCD, especially in those subjects who are homozygotes for the sickle cell substitution in HbS. Among the constitutional manifestations referred to herein by use of the term of SCD are delay of growth and development, an increased tendency to develop serious infections, particularly due to pneumococcus, marked impairment of splenic function, preventing effective clearance of circulating bacteria, with recurrent infarcts and eventual destruction of splenic tissue. Also involved in SCD are acute episodes of musculoskeletal pain, which affect primarily the lumbar spine, abdomen, and femoral shaft, and which are similar in mechanism and in severity. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia, and dehydration, all of which potentiate intracellular polymerization of HbS (J. H. Jandl, Blood: Textbook of Hematology, 2nd Ed., Little, Brown and Company, Boston, 1996, pages 544-545).

As used herein, “THAL” or “thalassemia” refers to a hereditary disorder characterized by defective production of hemoglobin. In one embodiment, the term encompasses hereditary anemias that occur due to mutations affecting the synthesis of hemoglobins. In other embodiments, the term includes any symptomatic anemia resulting from thalassemic conditions such as severe or β-thalassemia, thalassemia major, thalassemia intermedia, α-thalassemias such as hemoglobin H disease. β-thalassemias are caused by a mutation in the β-globin chain, and can occur in a major or minor form. In the major form of β-thalassemia, children are normal at birth, but develop anemia during the first year of life. The mild form of β-thalassemia produces small red blood cells. Alpha-thalassemias are caused by deletion of a gene or genes from the globin chain.

Where HbF is functional for carrying and delivering oxygen to tissues, the re-induction or de-repression of HbF expression provides an avenue for treating β-hemoglobinopathies. One approach as described herein targets BCL11A, which is responsible for repression of HbF expression. In various embodiments, an antibody or nucleic acid or vector encoding an antibody that specifically binds BCL11A and does not substantially cross-react with BCL11B, can be used to de-repress expression of the HbF subunit genes.

### Inhibition of BCL11A

In various embodiments, the activity of BCL11A is inhibited or decreased, e.g., by targeted protein degradation. By “decreases BCL11A activity” or “inhibits BCL11A activity” is meant that the amount of functional activity of BCL11A is at least 5% lower in a cell or cell population treated with the compositions and methods described herein, than a comparable, control cell or population, wherein BCL11A is not targeted for degradation. BCL11A activity in a treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or less relative to a control treated population in which BCL11A is not targeted for degradation. At a minimum, BCL11A activity can be assayed by determining the amount of BCL11A at the protein level, using techniques standard in the art. Alternatively, or in addition, BCL11A activity can be determined using a reporter construct, wherein the reporter construct is sensitive to BCL11A activity.

Alternatively, or in addition, BCL11A activity can be assayed by measuring fetal hemoglobin expression at the mRNA or protein level following treatment targeting BCL11A for degradation. Increased expression of endogenous HbF in adult cells treated or contacted with a construct targeting BCL11A for degradation is indicative of de-repression, and therefore reduced activity of BCL11A.

### Treatment of Hemoglobinopathies by Targeting BCL11A

BCL11A activity and/or expression has been shown to repress expression of fetal hemoglobin isoforms. Thus, as noted above, inhibition or degradation of BCL11A removes or reduces this repression and permits fetal hemoglobin isoforms to be re-induced, for example, in an adult. Increasing expression of the γ-globin genes can ameliorate hemoglobinopathies. Thus, in some embodiments, targeted degradation of BCL11A via introduction of a targeted protein degradation construct as described herein can treat a hemoglobinopathy.

In connection with contacting a cell with a targeted protein degradation construct aimed at BCL11A, “increasing the fetal hemoglobin levels” in a cell indicates that HbF is at least 5% higher in populations treated with the construct than in a comparable, control population, wherein no BCL11A targeting construct is present. It is preferred that the percentage of HbF expression in a BCL11A targeting construct-treated population is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a control treated population of comparable size and culture conditions. The term “control treated population” is used herein to describe a population of cells that has been treated with identical media, viral induction, nucleic acid sequences, temperature, confluency, flask size, pH, etc., without the BCL11A targeting construct. In one embodiment, any method known in the art can be used to measure an increase in fetal hemoglobin expression, e. g. Western Blot analysis of fetal γ-globin protein and PCR quantification of mRNA encoding fetal hemoglobin (e.g., HBG1 or HBG2 mRNAs).

It should be understood that complete inhibition of BCL11A activity is not required to derepress HbF expression enough for therapeutic benefit. As discussed above, HbF makes up about 2% of hemoglobin in adult humans, although the percentage varies. In one embodiment, derepression of HbF expression such that the amount of HbF expressed in adult erythroid cells is increased to at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of the total hemoglobin can provide therapeutic benefit. In another embodiment, an increase in HbF expression by at least 5-fold, at least 10-fold, at least 15-fold, at least 20-fold, at least 25-fold, at least 30-fold, at least 35-fold, at least 40-fold, at least 45-fold, at least 50 fold or more relative to baseline without a BCL11A inhibitor can provide therapeutic benefit.

### Fusion Proteins

In some embodiments, an antibody or antigen-binding fragment thereof, e.g., a single domain antibody or nanobody, is fused to another polypeptide sequence to provide additional functionality to the antibody or antigen-binding fragment thereof. As discussed herein above, fusion of an antibody or antigen-binding fragment thereof that binds TRIM10 or TRIM58 to an antibody or antigen-binding fragment thereof that binds an erythroid-specific or erythroid-enriched polypeptide can provide an avenue for targeted protein degradation of the erythroid-specific or erythroid enriched polypeptide in erythroid cells. Alternatively, a TRIM10 or TRIM58 polypeptide can be fused to an antibody or antigen-binding fragment thereof that specifically binds an erythroid-specific or erythroid-enriched polypeptide to provide a targeted protein degradation construct for that erythroid-specific or erythroid-enriched polypeptide.

One of ordinary skill in the art, given sequences encoding TRIM10, TRIM58, or encoding an antibody or antigen-binding fragment thereof that specifically binds TRIM10 or TRIM58, can generate a fusion construct that fuses the TRIM10 or TRIM58 polypeptide or TRIM10 or TRIM58-specific antibody sequence to a given fusion partner, e.g., antibody that binds a target for targeted protein degradation where the sequence for the fusion partner is known. Fusion can optionally include a peptide linker between the fusion partners. In one embodiment, the linker can be a serine/glycine rich linker. By way of example, sequences for TRIM10, TRIM58, and nanobodies that specifically bind BCL11A are provided herein.

### Expression and Delivery of Targeted Protein Degradation Constructs

In various embodiments, fusion proteins as described herein that target a protein of interest, e.g., an erythroid protein of interest, for degradation can be expressed from a vector as a recombinant polypeptide.

Sequences encoding a fusion protein as described herein can be contained in or expressed by a desired vector. Many such vectors useful for transferring exogenous genes into target mammalian cells are available. As used herein, a vector can be viral or non-viral. The term “vector” encompasses any genetic element that is capable of replication when associated with the proper control elements and that can transfer gene sequences to cells. A vector can include, but is not limited to, an expression vector, a plasmid, phage, transposon, cosmid, artificial chromosome, virus, virion, etc.

An expression vector can direct expression of a polypeptide (e.g., a fusion protein for targeted degradation of an erythroid-enriched or erythroid specific protein) from nucleic acid sequences contained therein linked to transcriptional regulatory sequences on the vector. The sequences expressed will often, but not necessarily, be heterologous to the cell. An expression vector can comprise additional elements, for example, the expression vector may have two replication systems, thus allowing it to be maintained in two organisms, for example in human or mammalian cells for expression and in a prokaryotic host for cloning and amplification. Expression refers to the cellular processes involved in producing RNA and/or proteins and as appropriate, secreting proteins, including where applicable, but not limited to, for example, transcription, transcript processing, translation and protein folding, modification and processing. “Expression products” include RNA transcribed from a gene and polypeptides obtained by translation of mRNA transcribed from a gene or gene construct.

Thus, in some embodiments, provided herein is a vector comprising a nucleic acid encoding a fusion protein for targeted degradation of an erythroid-enriched or erythroid specific protein as described herein. Typically, where introduction of the sequence encoding the fusion protein is desired, the vector is a viral vector which is an adeno-associated virus (AAV), a retrovirus, bovine papilloma virus, an adenovirus vector, a lentiviral vector, a vaccinia virus or a polyoma virus, among others.

In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences.

Retroviruses can be chosen as gene delivery vectors due to their ability to integrate their genes into the host genome, transferring a large amount of foreign genetic material, infecting a broad spectrum of species and cell types and for being packaged in special cell lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture medium. The medium containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of viral vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector as described herein includes “expression control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. A promoter sequence is a nucleotide region comprising a DNA regulatory sequence, wherein the regulatory sequence is typically derived from a gene which is capable of binding RNA polymerase and initiating transcription of a downstream (3′-direction) coding sequence. Transcription promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters. In some embodiments, the promoter can be a promoter for an erythroid-enriched or erythroid-specific gene, e.g., a hemoglobin gene.

In some embodiments, provided herein is a host cell transformed with a nucleic acid molecule encoding a fusion protein for targeted degradation of an erythroid-enriched or erythroid specific protein as described herein. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.” Where the sequence was introduced via a viral vector, the host cell can also be said to have been “transduced” with the sequence.

In instances, e.g., where the polypeptide will be used in vivo, e.g., therapeutically (or where mammalian post-translational modifications are or may be beneficial) it can be advantageous to produce the fusion polypeptide in mammalian cell culture, in order to, for example, produce a protein with mammalian-type post translational modifications, such as glycosylation patterns. In such embodiments, the host cells can be isolated from a mammalian subject selected from a group consisting of: a human, a horse, a dog, a cat, a mouse, a rat, a cow, a pig and a sheep. In some embodiments, the host cell is a human cell. In some embodiments, the host cell is a cell in culture. The cells can be obtained directly from a mammal (preferably human), or from a commercial source, or from tissue, or in the form for instance of cultured cells, prepared on site or purchased from a commercial cell source and the like. The cells can come from any organ including but not limited to the blood or lymphatic system, e.g., an hematopoietic cell, an erythroid cell or erythroid precursor, from muscles, any organ, gland, the skin, brain, lung, liver, kidney, etc., In some embodiments, the cells are selected from the group consisting of epithelial cells, neural cells, epidermal cells, keratinocytes, hematopoietic cells, melanocytes, chondrocytes, hepatocytes, B-cells, T-cells, erythrocytes, macrophages, monocytes, fibroblasts, muscle cells, vascular smooth muscle cells, splenocytes, pancreatic β cells, among others.

While introduction of nucleic acid encoding the fusion proteins as described herein to prokaryotic or mammalian cells can be used to prepare and isolate the polypeptides or fusion polypeptides for various uses, introduction to mammalian cells, e.g., via viral vectors can also be used therapeutically. In therapeutic embodiments, expression from a vector, e.g., a viral vector, e.g., an AAV vector as discussed above, can be used to introduce sequence encoding the fusion protein into a cell expressing the target protein (including, but not limited to BCL11A, ZNF410 LRF) to thereby promote degradation of the target protein. Such cells include, for example, erythroid cells or erythroid progenitor cells in which inhibition of BCL11A can induce expression of HbF.

In some embodiments, the host cell is a stem cell. As used herein, the term “stem cell” refers to an undifferentiated cell that can be induced to proliferate. The stem cell is capable of self-maintenance or self-renewal, meaning that with each cell division, one daughter cell will also be a stem cell. Stem cells can be obtained from embryonic, post-natal, juvenile, or adult tissue. Stem cells can be pluripotent or multipotent. The term “progenitor cell,” as used herein, refers to an undifferentiated cell derived from a stem cell, and is not itself a stem cell. Some progenitor cells can produce progeny that are capable of differentiating into more than one cell type. Stem cells include pluripotent stem cells, which can form cells of any of the body's tissue lineages: mesoderm, endoderm and ectoderm. Therefore, for example, stem cells can be selected from a human embryonic stem (ES) cell; a human inner cell mass (ICM)/epiblast cell; a human primitive ectoderm cell, a human primitive endoderm cell; a human primitive mesoderm cell; and a human primordial germ (EG) cell. Stem cells also include multipotent stem cells, which can form multiple cell lineages that constitute an entire tissue or tissues, such as but not limited to hematopoietic stem cells or neural precursor cells. Stem cells also include totipotent stem cells, which can form an entire organism. In some embodiment, the stem cell is a mesenchymal stem cell. The term “mesenchymal stem cell” or “MSC” is used interchangeably for adult cells which are not terminally differentiated, which can divide to yield cells that are either stem cells, or which, irreversibly differentiate to give rise to cells of a mesenchymal cell lineage, e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. In some embodiments, the stem cell is a partially differentiated or differentiating cell. In some embodiments, the stem cell is an induced pluripotent stem cell (iPSC), which has been reprogrammed or de-differentiated. Stem cells can be obtained from embryonic, fetal or adult tissues.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a hematopoietic progenitor cell), which in turn can differentiate into other types of precursor cells further down the pathway (such as an erythrocyte precursor), and then to an end-stage differentiated cell, such as an erythrocyte, which plays a characteristic role in a certain tissue type, and may or may not retain the capacity to proliferate further.

If so desired, viral vectors can also be targeted, e.g. to an erythroid cell or erythroid progenitor cell by manipulating the viral capsid to comprise or display a ligand for a myeloid cell-specific cell-surface molecule as known in the art.

“Hematopoietic stem or progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted with a vector is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiments, the hematopoietic stem or progenitor cell has at least one of the cell surface markers characteristic of hematopoietic progenitor cells: CD34+, CD59+, Thyl/CD90+, CD38lo/−, and C-kit/CD117+. Preferably, the hematopoietic progenitor cells have several of these markers.

In some embodiments, the hematopoietic progenitor cells of the erythroid lineage have the cell surface marker characteristic of the erythroid lineage: CD71 and Ter119.

It is contemplated that a construct or vector encoding a fusion protein as described herein can be introduced to target cells ex vivo, which are then re-introduced to a subject, either with or without expansion or selection for transformed cells prior to re-introduction. In some embodiments, the cells can be autologous to the subject. In others, the cells can be allogeneic to the subject. In such embodiments, it is optional to enrich a population of target cells, whether from peripheral blood or, for example, from bone marrow, for target hematopoietic cells or target erythroid progenitor or erythroid cells. Such enrichment can be performed on the basis of cell surface expression of hematopoietic progenitor cell markers or erythroid progenitor cell or erythroid cell markers, e.g., as noted above. In one embodiment of this aspect, and all other aspects, the hematopoietic progenitor is a cell of the erythroid lineage. Methods of isolating hematopoietic progenitor cells are well known in the art, e.g., by flow cytometric purification of CD34+ or CD133+ cells, microbeads conjugated with antibodies against CD34 or CD133, markers of hematopoietic progenitor cells. Commercial kits are also available, e.g., MACS® Technology CD34 MicroBead Kit, human, and CD34 MultiSort Kit, human, and STEMCELL™ Technology EasySep™ Mouse Hematopoietic Progenitor Cell Enrichment Kit. Similar approaches can be applied to isolate other sub-populations based on the specific markers they express. Cells transduced or transformed with vectors as described herein can be cultured for expansion and/or subjected to selection for expression of the exogenous sequence prior to (re) introduction to a subject. In other embodiments, such expansion or selection is not performed, and cells contacted with a vector are (re) introduced to a subject after contacting in vitro.

In other embodiments, the construct or vector, e.g., a viral vector (e.g., an AAV vector, among others) can be introduced to a subject to thereby deliver an expression construct to cells in vivo. Such delivery can inhibit the function of the protein targeted for degradation in the target hematopoietic progenitor, erythroid progenitor or erythroid cells. When the fusion protein introduced targets BCL11A, such expression can thereby increase HbF expression in the cells and/or their progeny.

In other embodiments, the degradation-targeting fusion protein can be produced, e.g., as a further fusion with a cell-penetrating peptide to thereby facilitate introduction of the degradation-targeting fusion protein to a target cell.

In some embodiments, the hematopoietic stem or progenitor cells described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein. Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of October 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

Somatic cells, as that term is used herein, refer to any cells forming the body of an organism, excluding germline cells. Every cell type in the mammalian body-apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, an hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

In some embodiments, cell-penetration peptides or cell-penetrating peptides (CPPs) can be used as a transmembrane drug delivery agent for delivery of degradation-targeting fusion polypeptide as described herein. CPPs are a class of small cationic peptides of at least 10, or at least 11, or at least 12, or at least 13, or at least 14, or at least 15, or at least 15, or at least 20, or at least 25, or at least 30 amino acids that can be used as transmembrane drug delivery agents through various forms of endocytosis for compounds including drugs, imaging agents, oligonucleotides, peptides and proteins. CPPs are also known as “protein transduction domains.” CPPs include but are not limited to the peptides Tat (e.g., HIV-derived CPP TAT (48-60)) and penetratin. Addition of a CPP to a fusion protein provides an option for introducing the fusion protein to a target cell.

Delivery of a fusion protein as described herein, or nucleic acids encoding them can include the use of lipid complexes or lipid nanoparticles complexed or loaded with the fusion protein or nucleic acid encoding same. As used herein, the term “nanoparticle” refers to particles that are on the order of about 10⁻⁹ or one to several billionths of a meter. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Non-limiting examples of lipid-based nanoparticles include, but are not limited to: a solid lipid nanoparticle (SLN; e.g., a nanoparticle comprising a single outer phospholipid layer and an inner core comprising a lipophilic substance, such as a therapeutic agent); a nanostructured lipid carrier (NLC; e.g., which comprises a mixture of solid crystalline lipids and liquid lipids); a microemulsion or a nanoemulsion, e.g., comprising a liquid lipid droplet; a cubosome (e.g., a liquid crystalline nano-structure formed from the cubic phase of lipids, such as monooleate, or any other amphiphilic macromolecules with the property to be dispersed into particles; such cubosomes can further comprise a stabilizer); a non-lamellar lipid nanoparticle, referring to a nanoparticle that does not comprise a lipid bilayer, but rather comprises non-lamellar liquid crystalline structures, such as cubic, hexagonal, and sponge phases (such a non-lamellar lipid nanoparticle can be particularly useful for controlled release formulations, e.g., for delivering inhaled drugs); or any combination thereof, or any other known structures in the art such as an ethasome, which is a lipid vesicular carrier comprising a relatively high percentage of ethanol. In some embodiments, the lipid-based nanoparticle comprises at least one phospholipid, at least one charged lipid, cholesterol, at least one membrane protein, and/or at least one nucleic acid or polypeptide construct for delivery to a cell. In some embodiments, the polypeptide or nucleic acid construct is linked to the at least one phospholipid, the at least one charged lipid, the cholesterol, or the at least one membrane protein of the lipid-based nanoparticle. See e.g., Naseri et al., Adv. Pharm. Bull. 2015 Sep. 5 (3): 305-313; Montenegro et al., Journal of Drug Delivery Science and Technology, Volume 32, Part B, April 2016, Pages 100-112; Barriga et al., Angew Chem Int Ed Engl. 2019 Mar. 4, 58 (10): 2958-2978; Chang et al., Advances in Colloid and Interface Science, Volume 222, August 2015, Pages 135-147; Abdulbaqi et al., Int J Nanomedicine. 2016 May 25, 11:2279-304; the contents of which are incorporated herein by reference in their entireties.

### Administration & Efficacy

As used herein, the terms “administering,” “introducing” and “transplanting” are used interchangeably in the context of the placement of an agent, e.g. a targeted protein degradation fusion protein or composition thereof as described herein into a subject, by a method or route which results in at least partial localization of the introduced agent (i.e., a fusion protein as described herein) at a desired site, such as an hematopoietic progenitor cell, erythroid progenitor cell or erythroid cell, among others, such that a desired effect(s) is produced. Where cells are introduced, the cells, e.g. hematopoietic progenitor cells, or their differentiated progeny can be administered by any appropriate route which results in delivery to a desired location in the subject where at least a portion of the implanted cells or components of the cells remain viable.

Modes of administration include injection, infusion and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In one embodiment, the agent (e.g., fusion protein or composition described herein) as described herein is administered systemically. The phrases “systemic administration,” “administered systemically”, “peripheral administration” and “administered peripherally” as used herein refer to the administration of a formulation (whether including cells expressing a fusion protein or composition described herein, or including nucleic acid or a vector encoding a fusion protein or composition described herein, or including an isolated fusion protein or composition described herein) other than directly into a target site, tissue, or organ, such that it enters, instead, the subject's circulatory system and, thus, is subject to metabolism and other like processes.

When provided prophylactically, an agent (e.g., a fusion protein or composition described herein) can be administered to a subject in advance of any symptom of a hemoglobinopathy, e.g., perinatally, prior to the switch or prior to the completion of the switch from fetal γ-globin to predominantly β-globin. Accordingly, the prophylactic administration of, e.g., a modified hematopoietic progenitor cell population can serve to prevent a hemoglobinopathy, as described herein.

When provided therapeutically, the agent is provided at (or after) the onset of a symptom or indication of a hemoglobinopathy, e.g., upon the onset of sickle cell disease or thalassemia.

In one embodiment, the term “effective amount” as used herein refers to the amount of an agent (e.g., a fusion protein or composition as described herein) needed to alleviate at least one or more symptom or marker of a hemoglobinopathy, and relates to a sufficient amount of a composition to provide the desired effect, e.g., treat a subject having a hemoglobinopathy. The term “therapeutically effective amount” therefore refers to an amount of an agent that is sufficient to promote a particular effect when administered to a typical subject, such as one who has or is at risk for a hemoglobinopathy. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

The efficacy of a treatment comprising an agent (e.g., a fusion protein or composition described herein) as described herein for the treatment of a hemoglobinopathy can be determined by the skilled clinician. However, a treatment is considered “effective treatment,” as the term is used herein, if any one or more of the signs or symptoms of a hemoglobinopathy is altered in a beneficial and/or statistically significant manner. For the avoidance of doubt, an improvement of at least 10% or more in a given sign, symptom or marker after treatment is considered effective treatment, and preferably improvement by at least 20%, at least 30%, at least 40%, at least 50%, at least 60%, at least 70%, at least 80%, at least 90%, at least 95% or more. Effective treatment expressly includes an increase in levels of HbF as that term is defined herein. Efficacy can also be measured by failure of an individual to worsen as assessed by hospitalization or need for medical interventions (e.g., progression of the disease is halted or at least slowed). Methods of measuring these indicators are known to those of skill in the art and/or described herein.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, an effective dose can be formulated in an appropriate animal model. The effects of any particular dosage can be monitored by a suitable bioassay, including, but not limited to measurement of HbF expression. The dosage can be determined by a physician and adjusted, as necessary, to suit observed effects of the treatment.

The agents described herein can be formulated, in some embodiments, with one or more additional therapeutic agents currently used to prevent or treat hemoglobinopathy, for example, hydroxyurea. The effective amount of such other agents depend on the amount of the agent/compositions provided herein in the formulation, the type of disorder or treatment, and other factors discussed above. These are generally used in the same dosages and with administration routes as used herein before or about from 1 to 99% of the heretofore employed dosages.

The dosage ranges for the agents or pharmaceutical compositions provided herein depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 μg/kg body weight to 100 μg/kg body weight. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the hemoglobinopathy is treated, as measured by the methods described above or known in the art.

However, other dosage regimens can be useful. The duration of a therapy using the methods described herein will continue for as long as medically indicated or until a desired therapeutic effect (e.g., those described herein) is achieved. In certain embodiments, the administration of the pharmaceutical composition described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

As will be appreciated by one of skill in the art, appropriate dosing regimens for a given composition can comprise a single administration or multiple ones. Subsequent doses may be given repeatedly at time periods, for example, about two weeks or greater up through the entirety of a subject's life, e.g., to provide a sustained therapeutic or preventative effect. The precise dose to be employed in the formulation will also depend on the route of administration and should be decided according to the judgment of the practitioner and each patient's circumstances. Ultimately, the practitioner or physician will decide the amount of the agent or composition thereof to administer to particular subjects.

The treatment as described herein ameliorates one or more symptoms associated with, e.g., a β-globin disorder by increasing the amount of fetal hemoglobin in the individual. Symptoms typically associated with a hemoglobinopathy include, for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

The levels of BCL11A or fetal hemoglobin (HbF) can be determined by methods known in the art. For example, PCR, Western blotting, immunological methods, flow cytometric analyses, ELISA. Accordingly, the activity of BCL11A can be determined by methods known in the art, e.g., a chromatin occupancy assay, binding assays, pull-down assays, RT-PCR of fetal hemoglobin levels, animal models, etc. BCL11A activity can be assayed by measuring fetal hemoglobin expression at the mRNA or protein level following treatment with a polypeptide, nucleic acid molecule, vector, nanoparticle or other agent providing a BCL11A-specific single domain antibody or construct as described herein.

In some embodiments of any of the aspects, the level or activity of BCL11A is decreased by at least 10%, at least 20%, at least 30%, at least 40%, at least 50%, at least 60%, at least 70%, at least 80%, at least 90%, or more as compared to an appropriate control. In some embodiments, a decreased level or activity of BCL11A, in a cell of the subject, increases the level and/or activity of fetal hemoglobin (HbF) by at least 10%, at least 20%, at least 30%, at least 40%, at least 50%, at least 60%, at least 70%, at least 80%, at least 90%, or more as compared to an appropriate control.

The technology provided herein can further be defined by the following numbered paragraphs.

Paragraph 1: A fusion polypeptide comprising a binding domain that specifically binds an erythroid-enriched E3 ubiquitin ligase and a binding domain that specifically binds an erythroid-enriched polypeptide of interest.

Paragraph 2: The fusion polypeptide of paragraph 1 which mediates degradation of the polypeptide of interest in erythroid cells.

Paragraph 3: The fusion polypeptide of any of the preceding paragraphs, wherein the binding domain that specifically binds an erythroid-enriched E3 ubiquitin ligase specifically binds TRIM10 or TRIM58.

Paragraph 4: The fusion polypeptide of any of the preceding paragraphs, wherein the binding domain that specifically binds an erythroid-enriched E3 ubiquitin ligase comprises an antibody or antigen-binding fragment thereof.

Paragraph 5: The fusion polypeptide of any of the preceding paragraphs, wherein the antibody or antigen-binding fragment thereof comprises an scFv, a single domain antibody or a nanobody.

Paragraph 6: The fusion polypeptide of any of the preceding paragraphs, wherein the binding domain that specifically binds an erythroid-enriched polypeptide of interest comprises an antibody or antigen-binding fragment thereof.

Paragraph 7: The fusion polypeptide of any of the preceding paragraphs, wherein the antibody or antigen-binding fragment thereof comprises an scFv, a single domain antibody or a nanobody.

Paragraph 8: The fusion polypeptide of any of the preceding paragraphs, wherein the polypeptide of interest is selected from BCL11A, LRF and ZNF410.

Paragraph 9: The fusion polypeptide of any of the preceding paragraphs, wherein the E3 ubiquitin ligase is TRIM10 or TRIM58.

Paragraph 10: The fusion polypeptide of any of the preceding paragraphs, wherein the polypeptide of interest is BCL11A, and the binding domain that specifically binds the polypeptide of interest binds BCL11A at an epitope comprised by zinc-finger 6 (ZNF6) of the BCL11A polypeptide.

Paragraph 11: The fusion polypeptide of any of the preceding paragraphs, wherein the polypeptide of interest is BCL11A, and the binding domain that specifically binds the polypeptide of interest binds BCL11A at an epitope comprised by zinc finger 23 (ZNF23) of the BCL11A polypeptide.

Paragraph 12: The fusion polypeptide of any of the preceding paragraphs, wherein the polypeptide of interest is BCL11A, and the binding domain that specifically binds the polypeptide of interest has an amino acid sequence at least 90% identical to SEQ ID NO: 12.

Paragraph 13: The fusion polypeptide of any of the preceding paragraphs, wherein amino acid sequence variation relative to SEQ ID NO: 12 occurs at one or more of amino acids according to Table 2.

Paragraph 14: The fusion polypeptide of any of the preceding paragraphs, wherein the polypeptide of interest is BCL11A, and the binding domain that specifically binds BCL11A has an amino acid sequence at least 90% identical to SEQ ID NO: 34.

Paragraph 15: The fusion polypeptide of any of the preceding paragraphs, wherein amino acid sequence variation relative to SEQ ID NO: 34 occurs at one or more of amino acids at amino acid number 102 or 108.

Paragraph 16: The fusion polypeptide of any of the preceding paragraphs, wherein the polypeptide of interest is BCL11A, and the binding domain that specifically binds BCL11A comprises a single-domain antibody in which:
CDR 1 has an amino acid sequence selected from SEQ ID NOs: 43-50,
CDR2 has an amino acid sequence selected from SEQ ID NOs: 51-59; and
CDR3 has an amino acid sequence selected from SEQ ID NOs 60-65.

Paragraph 17: The fusion polypeptide of any of the preceding paragraphs, wherein the polypeptide of interest is BCL11A, and the binding domain that specifically binds BCL11A comprises a single-domain antibody in which:

CDR1 has the amino acid sequence
(SEQ ID NO: 37)
SIFVNNAM;
and
CDR2 has the amino acid sequence
(SEQ ID NO: 38)
ELVAAISASGGSTYY;
CDR3 has a sequence selected from
(SEQ ID NO: 39)
ADQDVYPYEYW,
(SEQ ID NO: 40)
ADQDGYPYEYW,
and
(SEQ ID NO: 41)
ADQDVYPYEYL.

Paragraph 18: The fusion polypeptide of any of the preceding paragraphs which further comprises a cell-penetrating peptide.

Paragraph 19: The fusion polypeptide of any of the preceding paragraphs, further comprising a linker peptide between the binding domain that specifically binds an erythroid-enriched E3 ubiquitin ligase and the binding domain that specifically binds an erythroid-enriched polypeptide of interest.

Paragraph 20: A nucleic acid comprising sequence encoding a fusion polypeptide of any of the preceding paragraphs.

Paragraph 21: A nucleic acid of any of the preceding paragraphs, wherein the sequence encoding the fusion polypeptide is operatively linked to regulatory sequences that permit expression in erythroid cells.

Paragraph 22: A vector comprising a nucleic acid of any of the preceding paragraphs.

Paragraph 23: The vector of any of the preceding paragraphs, which is a viral vector.

Paragraph 24: The viral vector of any of the preceding paragraphs, which is an AAV vector.

Paragraph 25: A method of erythroid-specific, targeted degradation of a protein of interest, the method comprising introducing a fusion polypeptide of any of the preceding paragraphs, a nucleic acid of any of the preceding paragraphs, or a vector of any of the preceding paragraphs to an erythroid cell.

Paragraph 26: A method of targeted degradation of BCL11A, the method comprising introducing a fusion polypeptide of any of the preceding paragraphs, a nucleic acid of any of the preceding paragraphs, or a vector of any of the preceding paragraphs to a cell expressing BCL11A.

Paragraph 27: The method of any of the preceding paragraphs, wherein the targeted degradation of BCL11A is targeted to erythroid cells.

Paragraph 28: A method of promoting fetal hemoglobin (HbF) expression in adult erythroid cells, the method comprising introducing a fusion polypeptide of any of the preceding paragraphs, a nucleic acid of any of the preceding paragraphs, or a vector of any of the preceding paragraphs to a cell, wherein the protein of interest is BCL11A, and wherein the fusion polypeptide promotes the degradation of BCL11A in erythroid cells, thereby promoting HbF expression.

Paragraph 29: A method of treating a hemoglobinopathy disorder, the method comprising introducing a fusion polypeptide of any of the preceding paragraphs, a nucleic acid of any of the preceding paragraphs, or a vector of any of the preceding paragraphs to a subject in need thereof, wherein the protein of interest is BCL11A, and wherein the fusion polypeptide promotes the degradation of BCL11A in erythroid cells, thereby promoting HbF expression to treat the hemoglobinopathy disorder.

Paragraph 30: A fusion polypeptide comprising a TRIM10 or TRIM58 polypeptide fused to a binding domain that specifically binds a target polypeptide.

Paragraph 31: The fusion polypeptide of claim 30, wherein the binding domain that specifically binds a target polypeptide comprises an antigen-binding domain of an antibody.

Paragraph 32: The fusion polypeptide of any of the preceding paragraphs, wherein the binding domain that specifically binds a target polypeptide comprises an scFv, a single domain antibody or a nanobody.

Paragraph 33: The fusion polypeptide of any of the preceding paragraphs, wherein the target polypeptide is expressed in an erythroid cell.

Paragraph 34: The fusion polypeptide of any of the preceding paragraphs, wherein the target polypeptide is selected from BCL11A, LRF and ZNF410.

Paragraph 35: The fusion polypeptide of any of the preceding paragraphs, wherein the polypeptide of interest is BCL11A, and the binding domain that specifically binds the polypeptide of interest binds BCL11A at an epitope comprised by zinc-finger 6 (ZNF6) of the BCL11A polypeptide.

Paragraph 36: The fusion polypeptide of any of the preceding paragraphs, wherein the polypeptide of interest is BCL11A, and the binding domain that specifically binds the polypeptide of interest binds BCL11A at an epitope comprised by zinc finger 23 (ZNF23) of the BCL11A polypeptide.

Paragraph 37: The fusion polypeptide of any of the preceding paragraphs, wherein the polypeptide of interest is BCL11A, and the binding domain that specifically binds the polypeptide of interest has an amino acid sequence at least 90% identical to SEQ ID NO: 12.

Paragraph 38: The fusion polypeptide of claim 37, wherein amino acid sequence variation relative to SEQ ID NO: 12 occurs at one or more of amino acids according to Table 2.

Paragraph 39: The fusion polypeptide of any of the preceding paragraphs, wherein the polypeptide of interest is BCL11A, and the binding domain that specifically binds BCL11A has an amino acid sequence at least 90% identical to SEQ ID NO: 34.

Paragraph 40: The fusion polypeptide of any of the preceding paragraphs, wherein amino acid sequence variation relative to SEQ ID NO: 34 occurs at one or more of amino acids number 102 or 108.

Paragraph 41: The fusion polypeptide of any of the preceding paragraphs, wherein the polypeptide of interest is BCL11A, and the binding domain that specifically binds BCL11A comprises a single-domain antibody in which:
CDR1 has an amino acid sequence selected from SEQ ID NOs: 43-50,
CDR2 has an amino acid sequence selected from SEQ ID NOs: 51-59; and
CDR3 has an amino acid sequence selected from SEQ ID NOs 60-65.

Paragraph 42: The fusion polypeptide of any of the preceding paragraphs, wherein the polypeptide of interest is BCL11A, and the binding domain that specifically binds BCL11A comprises a single-domain antibody in which:

CDR1 has the amino acid sequence
(SEQ ID NO: 37)
SIFVNNAM;
and
CDR2 has the amino acid sequence
(SEQ ID NO: 38)
ELVAAISASGGSTYY;
CDR3 has a sequence selected from
(SEQ ID NO: 39)
ADQDVYPYEYW,
(SEQ ID NO: 40)
ADQDGYPYEYW,
and
(SEQ ID NO: 41)
ADQDVYPYEYL.

Paragraph 43: The fusion polypeptide of any of the preceding paragraphs which further comprises a cell-penetrating peptide.

Paragraph 44: The fusion polypeptide of any of the preceding paragraphs, further comprising a linker peptide between the TRIM10 or TRIM58 polypeptide and the binding domain that specifically binds a target polypeptide.

Paragraph 45: A nucleic acid comprising sequence encoding a fusion polypeptide of any one of any of the preceding paragraphs.

Paragraph 46: The nucleic acid of any of the preceding paragraphs, wherein the sequence encoding the fusion polypeptide is operatively linked to regulatory sequences that permit expression in erythroid cells.

Paragraph 47: A vector comprising a nucleic acid of any of the preceding paragraphs.

Paragraph 48: The vector of any of the preceding paragraphs, which is a viral vector.

Paragraph 49: The viral vector of claim 48, which is an AAV vector.

Paragraph 50: A method of erythroid-specific, targeted degradation of a protein of interest, the method comprising introducing a fusion polypeptide of any of the preceding paragraphs, a nucleic acid of any of the preceding paragraphs, or a vector of any of the preceding paragraphs to an erythroid cell.

Paragraph 51: A method of targeted degradation of BCL11A, the method comprising introducing a fusion polypeptide of any of the preceding paragraphs, a nucleic acid of any of the preceding paragraphs, or a vector of any of the preceding paragraphs to a cell expressing BCL11A.

Paragraph 52: The method of any of the preceding paragraphs, wherein the targeted degradation of BCL11A is targeted to erythroid cells.

Paragraph 53: A method of promoting fetal hemoglobin (HbF) expression in adult erythroid cells, the method comprising introducing a fusion polypeptide of any of the preceding paragraphs, a nucleic acid of any of the preceding paragraphs, or a vector of any of the preceding paragraphs to a cell, wherein the protein of interest is BCL11A, and wherein the fusion polypeptide promotes the degradation of BCL11A in erythroid cells, thereby promoting HbF expression.

Paragraph 54: A method of treating a hemoglobinopathy disorder, the method comprising introducing a fusion polypeptide of any of the preceding paragraphs, a nucleic acid of claim 45 or 46, or a vector of any of the preceding paragraphs to a subject in need thereof, wherein the protein of interest is BCL11A, and wherein the fusion polypeptide promotes the degradation of BCL11A in erythroid cells, thereby promoting HbF expression to treat the hemoglobinopathy disorder.

### EXAMPLES

### Example 1

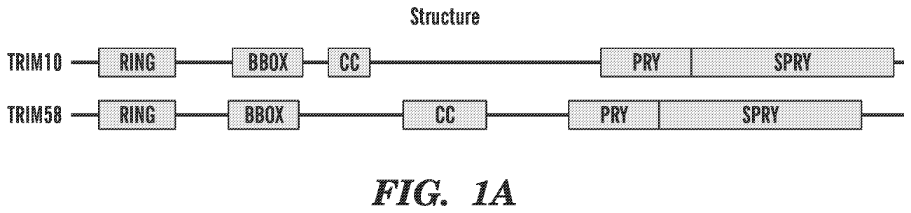

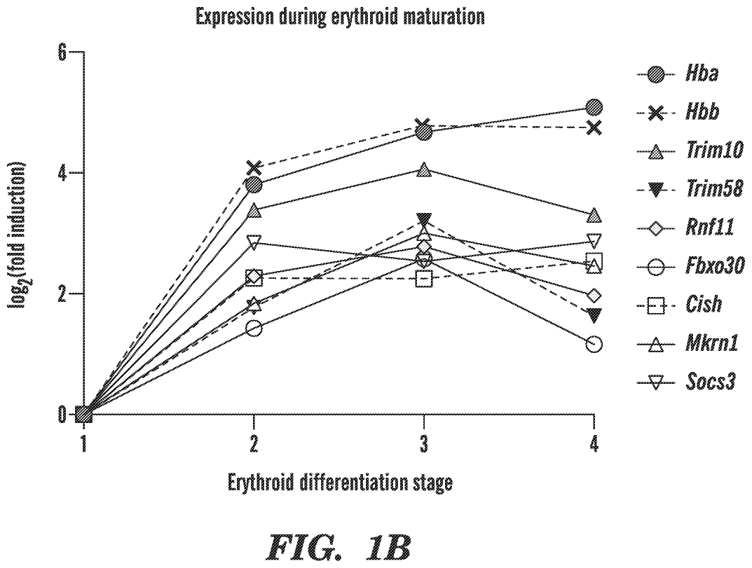

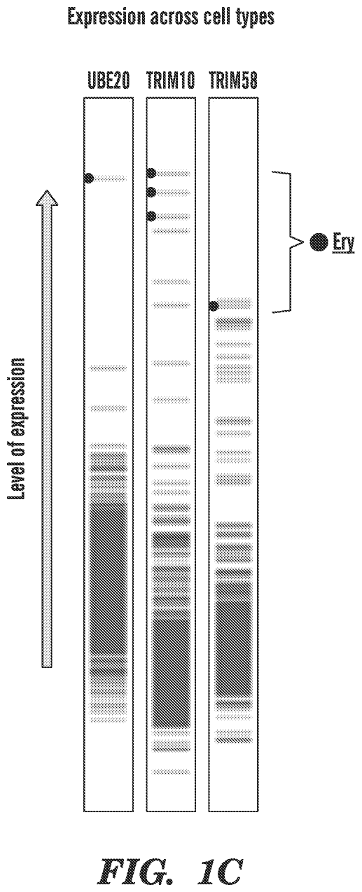

As described herein, the invention comes in part from investigation into erythroid specific E3 ubiquitin ligases. Analysis of RNA expression of E3 ligases during erythroid cell maturations revealed specific expression of TRIM10 and TRIM58 during erythroid cell maturation (FIG. 1B). TRIM10 and TRIM58 are members of the tripartite motif family that includes three zinc-binding domains, a RING, a B-box type 1 and a B-box type 2, and a coiled-coil region (FIG. 1A). Furthermore, TRIM10 and TRIM58 were found to be enriched in erythroid cells as they are highly expressed in that cell type but expressed at much lower levels across other cell types. Known erythroid cell specific E3 ligase UBE20 was used as a positive control (FIG. 1C).

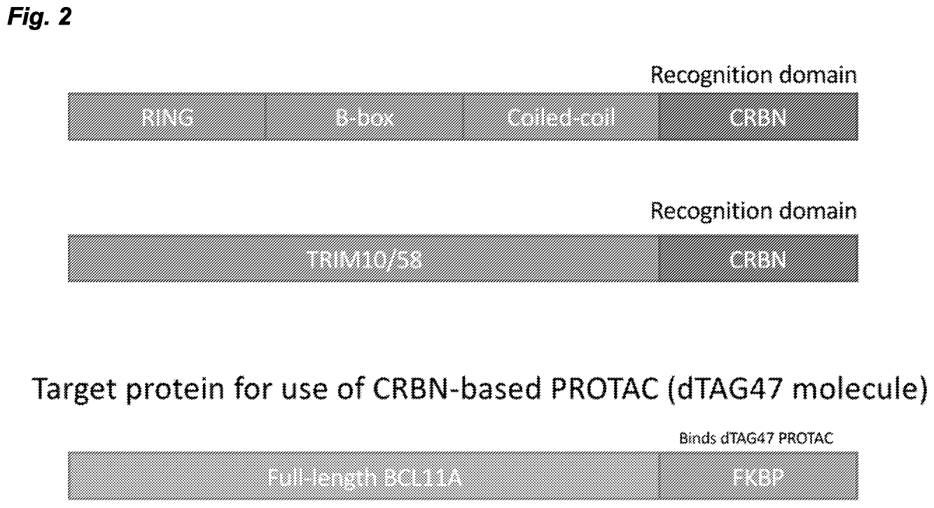

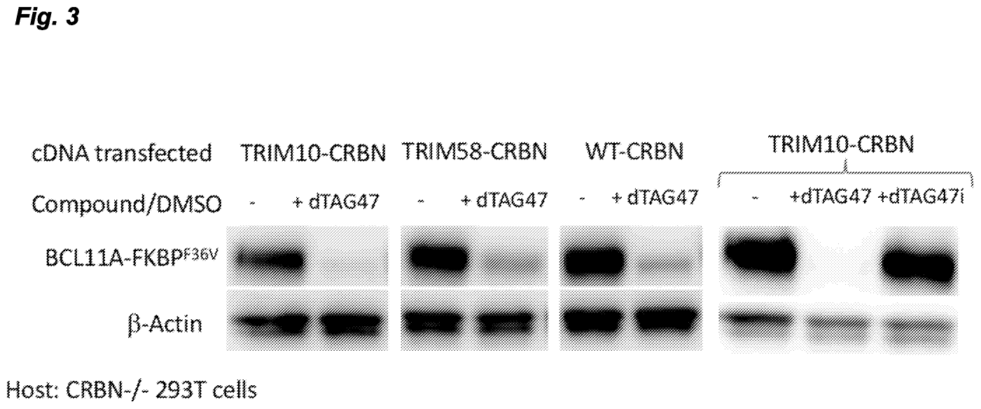

In order to utilize erythroid specific E3 ubiquitin ligases for targeted protein degradation, proteolysis-targeting chimeras were created using TRIM10 or TRIM58. More specifically, TRIM10 or TRIM58 were fused to the recognition domain of cereblon (CRBN), then CRBN^{-/-}HEK293T cells were co-transfected with the E3 ligase construct and BCL11A tagged with FKBP, which binds to dTAG47 PROTAC (a heterobifuncational molecule including a thalidomide derivative and an FKBP12F36V-selective ligand) (FIG. 2) Following transfection, dTAG47 was added to cells for 48 hrs and cells were collected via fluorescent activated cell sorting (FACS). To determine if TRIM-CRBN fusion can degrade BCL11A-FKBP, Western blot analysis with BCL11A antibody was used. Results indicate that fusions of TRIM10/58 with the recognition domain of CRBN are capable of degrading tagged BCL11A upon addition of dTAG47 PROTAC. Wild-type CRBN (WT-CRBN) cDNA transfected into CRBN—-293T cells was used as a positive control. An inactive form of dTAG47 (dTAG47i) is inactive for degradation and showed no effects, indicating that degradation is dependent on recruitment through the CRBN recognition domain (FIG. 3).

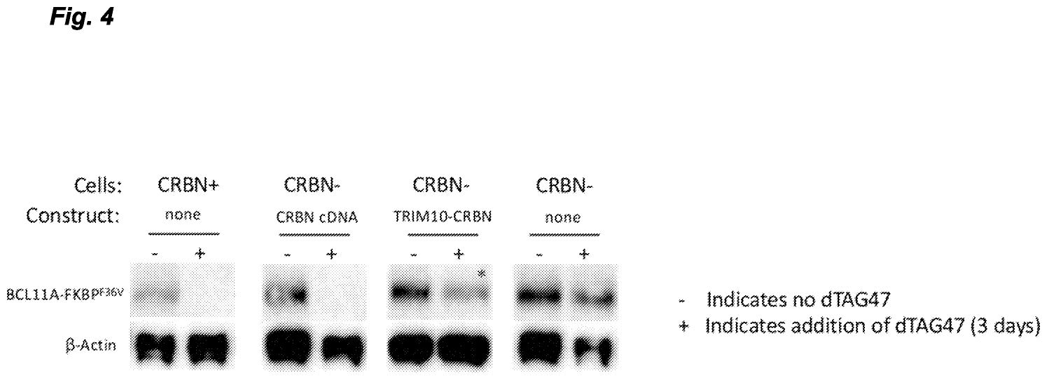

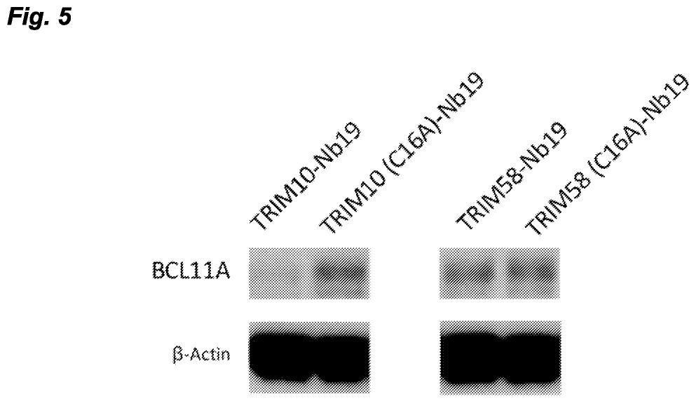

Next, the degradation of BCL11A protein by TRIM10/58 in HUDEP-2 (erythroid cells) were tested. HUDEP-2 cells were tagged with FKBP, which permits the use of dTAG47 PROTAC for recruitment to CRBN for degradation. As a control, HUDEP-2 cells lacking CRBN were generated by CRISPR/Cas9 editing. CRBN+ and CRBN-cells were infected with lentiviruses expressing CRBN cDNA or TRIM10 fused with the recognition domain of CRBN and cells were cultured with and without dTAG47 PROTAC. Degradation of BCL11A was analyzed by Western blot using BCL11A antibody. The fusion of TRIM10 with the recognition domain of CRBN led to a reduction in BCL11A. In CRBN-cells, degradation of BCL11A does not occur (FIG. 4). These results indicate that when recruited to BCL11A by the recognition domain of CRBN, TRIM10 is capable of degrading BCL11A. In other experiments, no degradation is seen with these cells in the presence of dTAG47.

Finally, it was investigated whether BCL11A could be targeted for degradation by TRIM10/58 using BCL11A nanobodies. In these experiments, 293T cells were transfected with constructs expressing either TRIM10 or TRIM58 fused to a BCL11A-specific nanobody. Analysis by Western blot with BCL11A allowed for detection of endogenous BCL11A. As a control, mutant versions of TRIM10 or TRIM58 (C16A) in which a critical, conserved residue of TRIM proteins is mutated were used. Cells were transfected and assayed 2 days later. Results show marked reduction in BCL11A in cells treated with TRIM10-Nb19 (BCL11A) fusion. Thus, TRIM10 and to a lesser extent TRIM58 are able to direct degradation of wild-type, untagged BCL11A protein.

1. A fusion polypeptide comprising a binding domain that specifically binds an erythroid-enriched E3 ubiquitin ligase and a binding domain that specifically binds an erythroid-enriched polypeptide of interest.

2. The fusion polypeptide of claim 1 which mediates degradation of the polypeptide of interest in erythroid cells.

3. The fusion polypeptide of claim 1, wherein the binding domain that specifically binds an erythroid-enriched E3 ubiquitin ligase specifically binds TRIM10 or TRIM58.

4. The fusion polypeptide of claim 1, wherein the binding domain that specifically binds an erythroid-enriched E3 ubiquitin ligase comprises an antibody or antigen-binding fragment thereof.

5. The fusion polypeptide of claim 4, wherein the antibody or antigen-binding fragment thereof comprises an scFv, a single domain antibody or a nanobody.

6. The fusion polypeptide of claim 1, wherein the binding domain that specifically binds an erythroid-enriched polypeptide of interest comprises an antibody or antigen-binding fragment thereof.

7. The fusion polypeptide of claim 6, wherein the antibody or antigen-binding fragment thereof comprises an scFv, a single domain antibody or a nanobody.

8. The fusion polypeptide of claim 1, wherein the polypeptide of interest is selected from BCL11A, LRF and ZNF410.

9. The fusion polypeptide of claim 1, wherein the E3 ubiquitin ligase is TRIM10 or TRIM58.

10. The fusion polypeptide of claim 1, wherein the polypeptide of interest is BCL11A, and the binding domain that specifically binds the polypeptide of interest binds BCL11A at an epitope comprised by zinc-finger 6 (ZNF6) of the BCL11A polypeptide or by zinc finger 23 (ZNF23) of the BCL11A polypeptide.

11. (canceled)

12. The fusion polypeptide of claim 1, wherein the polypeptide of interest is BCL11A, and the binding domain that specifically binds the polypeptide of interest has an amino acid sequence at least 90% identical to SEQ ID NO: 12.

13. The fusion polypeptide of claim 12, wherein amino acid sequence variation relative to SEQ ID NO: 12 occurs at one or more of amino acids according to Table 2.

14. The fusion polypeptide of claim 1, wherein the polypeptide of interest is BCL11A, and the binding domain that specifically binds BCL11A has an amino acid sequence at least 90% identical to SEQ ID NO: 34.

15. The fusion polypeptide of claim 14, wherein amino acid sequence variation relative to SEQ ID NO: 34 occurs at one or more of amino acids at amino acid number 102 or 108.

16. The fusion polypeptide of claim 1, wherein the polypeptide of interest is BCL11A, and the binding domain that specifically binds BCL11A comprises a single-domain antibody in which:
CDR1 has an amino acid sequence selected from SEQ ID NOs: 43-50,
CDR2 has an amino acid sequence selected from SEQ ID NOs: 51-59; and
CDR3 has an amino acid sequence selected from SEQ ID NOs 60-65.

17. The fusion polypeptide of claim 1, wherein the polypeptide of interest is BCL11A, and the binding domain that specifically binds BCL11A comprises a single-domain antibody in which:
CDR1 has the amino acid sequence
(SEQ ID NO: 37)
SIFVNNAM;
and
CDR2 has the amino acid sequence
(SEQ ID NO: 38)
ELVAAISASGGSTYY;
CDR3 has a sequence selected from
(SEQ ID NO: 39)
ADQDVYPYEYW,
(SEQ ID NO: 40)
ADQDGYPYEYW,
and
(SEQ ID NO: 41)
ADQDVYPYEYL.

18. The fusion polypeptide of claim 1, which further comprises a cell-penetrating peptide.

19. The fusion polypeptide of claim 18, further comprising a linker peptide between the binding domain that specifically binds an erythroid-enriched E3 ubiquitin ligase and the binding domain that specifically binds an erythroid-enriched polypeptide of interest.

20. A nucleic acid comprising sequence encoding a fusion polypeptide of claim 1.

21. (canceled)

22. A vector comprising a nucleic acid of claim 20.

23-24. (canceled)

25. A method of erythroid-specific, targeted degradation of a protein of interest, the method comprising introducing a fusion polypeptide of claim 1 to an erythroid cell.

26-29. (canceled)

30. A fusion polypeptide comprising a TRIM10 or TRIM58 polypeptide fused to a binding domain that specifically binds a target polypeptide.

31-54. (canceled)


SEQUENCE LISTING (72 sequences)

SEQ ID NO: 1 - protein, 76 aa, unidentified [sequence omitted]

SEQ ID NO: 2 - RNA, 3825 nt, Homo sapiens
  source 1..3825: mol_type = mRNA
cttctgacagtggacagctgacatatcaccactttccttctactgtgagtgtctctggatgggcagaaaggaatggccagcccctggttatggtcatctaaggtcacctctgaaatgctgtgagcccctcttccttcctctcctctgctatttcccatctctgctgttggcaggagaatagaaccctggctgccagagatgcaagtgtgtgacgatatgggtgctggtgcatatttagtatgtgcctgtgtccagccatgtgcatgtgtgggtgtgtgagtgtgtgacccagcccttcccccgtggccaagcagagagagtggccttgaggaagccatagcagcaggaccagcatggcctctgctgcctctgtgaccagcctggcagatgaagtcaactgccccatctgtcagggtaccctgagggagccggtcactatcgactgcggccacaacttctgccgggcctgccttacccgctactgtgagataccaggcccagacctggaggagtcccctacttgcccactctgcaaagaacccttccgtcctgggagcttccggcccaactggcagctggctaacgtggtggagaacattgagcgcctccagctggtgtccacactgggtttgggagaggaggatgtctgccaagagcacggagagaagatctacttcttctgtgaggatgatgagatgcagttgtgcgtggtgtgccgggaggctggggagcacgctacccacaccatgcgcttcctggaggatgcagcggctccctatagggaacaaatccataagtgtcttaaatgtctaagaaaagagagagaggagattcaagaaatccagtcaagagaaaataaaaggatgcaagtcctcctgactcaggtgtccaccaagagacaacaggtgatttctgagttcgcacacctgaggaagtttctagaggaacagcagagcatcctcttagcacaattggagagccaggatggggacatcttgaggcaacgggatgaatttgatttgctggttgctggggagatctgccggtttagtgctcttattgaagaactggaggagaagaatgagaggccagcaagggagctcctgacggacatcagaagcactctaataagatgtgaaaccagaaagtgccggaaaccggtggctgtgtcgccagagctgggccagaggattcgggactttccccagcaggccctcccgctgcagagggagatgaagatgtttctggaaaaactatgctttgagttggactatgagccagctcacatttctctagaccctcagacttcccaccccaagctcctcttgtccgaggaccaccagcgagctcagttctcctacaaatggcagaactcaccagacaacccccaacgttttgaccgggccacctgtgttctggcccacactggcatcacaggggggagacacacgtgggtggtgagtatagacctggcccatgggggcagctgcaccgtgggcgtggtgagcgaggatgtgcagcggaagggggagcttcggctgcggccagaggagggggtgtgggctgtgaggctggcttggggcttcgtctcggctctgggctccttccccacacggctgaccctgaaggagcagccccggcaggtgagggtgtctcttgactatgaggtgggctgggtgaccttcaccaacgctgtcacccgagagcccatctacaccttcactgcctccttcactaggaaggtcattcccttctttgggctctggggccgagggtccagtttctccctgagctcctgagaaggagcagttacctactctcctctaagtacaggactcatatcaacccaagtaccatgtggacttgatccctggctgaatcacctggatgacttggaatagaaatgactgctttagaagatgggatggggccgggtggtaagggatagaagagaggactctcaatctactgatcaagtcctttccccaatgcccagtggatggccagggtacctggggactcaggctgctgccagttctgctcaccaccatccgtgcttggcacagaagtagctgcatagaaagggcactggatttgaagtcagaagacctgggttcttgaaccagcctgtcaaccagttgtatgactttaaacaaggcatctcacctcttttcatcttgttttcttccaataatgttagagttcatgtaatcacattctctagaaccatttagtttgtgttaactatgaaccaagcagtgtggtggccactggtgacttgaaaatatagagaaaaaaaaaacctgctctatatctgaaagagctcttgggaagacagagaaacataaagaggaaattacagcacagtgtggtgggtgttacgggaagtccaaccccagcattatgggagttcaggggaaggggcatagcccagcctggaaggagagggtgagtgggggatggctttctgaaaagggtggtctaaaggatgcctatggtcaatagggaaaagaggggaagaagcatcttaagaagaggaaacagcagagaactggctggatgacctgtgactctggagcactgggttgtctccattgtcattatgggcagatgtgtgccatccccagccgacgccactcactgcctccttcctcctggtgttgccacttctgggtgagattaaggtgcagggcctgggggcaggaggacataaggtatagccataaatcataacccagggaccacactcaaccctagggaaattgtcttcctgatcagttgattaccatctgaggtcaagaaatgagatagtgggagcaaatgggtcacaaatagctcagctgtgggctcagaaactgctaggtaaaagaattccagaaggaggccagggcataagttggatgacctatgaactttagtctaaagaattgagactaccgtaattgagactactgtagtgacatctgagaaatgggatggaagagtgaccatgttttattttcttgttcttgtcactattgtattttattttgcataatcatgcccttcactgacagtctccttaacatcatctgtttactctgctcagtgtaaactacaatgctctgtcatctccctactgggtctcctgggaggaggggagccatccagggtgcaaactcaaaggcagagggcacagcgtgcttaggcccaagcttagaattcaattgagaagttctgttgttcatccttacctcagcaggtagaaaagaggtggagatcagaagccagggattagagattgaattgctttccctgggagtgtgcagtatctcattaaaatgttgtatattcaaaaaaatacagacacacacaagtgcctatataatgataaaacatatccaaagcacccaaacctgtgattccagaaaagtagatcctatttttgtttatttttattggctttagctgtgtcttttgaaaggactattatcctctaaaatgtatgtgtatgaagatcctggcttgcagctcgggagctttacacaccttgtgtcactttatcttggtaacgagccaagatcatgcaaccagttcatccatgtctgacccaagagctcttaactgctatgctgcactgcctcattcagagatagatgcctgcatgggtcctggcgattattttaatgctggctacacccccacaggtgacttggattcagaaatacaatttatatttcttctttttaaattgttttattttatttttcttttaatagttatatgtcagtgagaacaatttatatgtcttacactgagaaataaaactgctcataagtgaaaaca

SEQ ID NO: 3 - protein, 481 aa, Homo sapiens
MASAASVTSLADEVNCPICQGTLREPVTIDCGHNFCRACLTRYCEIPGPDLEESPTCPLCKEPFRPGSFRPNWQLANVVENIERLQLVSTLGLGEEDVCQEHGEKIYFFCEDDEMQLCVVCREAGEHATHTMRFLEDAAAPYREQIHKCLKCLRKEREEIQEIQSRENKRMQVLLTQVSTKRQQVISEFAHLRKFLEEQQSILLAQLESQDGDILRQRDEFDLLVAGEICRFSALIEELEEKNERPARELLTDIRSTLIRCETRKCRKPVAVSPELGQRIRDFPQQALPLQREMKMFLEKLCFELDYEPAHISLDPQTSHPKLLLSEDHQRAQFSYKWQNSPDNPQRFDRATCVLAHTGITGGRHTWVVSIDLAHGGSCTVGVVSEDVQRKGELRLRPEEGVWAVRLAWGFVSALGSFPTRLTLKEQPRQVRVSLDYEVGWVTFTNAVTREPIYTFTASFTRKVIPFFGLWGRGSSFSLSS

SEQ ID NO: 4 - RNA, 5170 nt, Homo sapiens
  source 1..5170: mol_type = mRNA
gcagaccgcgaggggagacggtgcgggcggccgggagcgcagccctccgggaggcgggtcatggcctgggcgccgcccggggagcggctgcgcgaggatgcgcggtgcccggtgtgcctggatttcctgcaggagccggtcagcgtggactgcggccacagcttctgcctcaggtgcatctccgagttctgcgagaagtcggacggcgcgcagggcggcgtctacgcctgtccgcagtgccggggccccttccggccctcgggctttcgccccaaccggcagctggcgggcctggtggagagcgtgcggcggctggggttgggcgcggggcccggggcgcggcgatgcgcgcggcacggcgaggacctgagccgcttctgcgaggaggacgaggcggcgctgtgctgggtgtgcgacgccggccccgagcacaggacgcaccgcacggcgccgctgcaggaggccgccggcagctaccaggtaaagctccagatggctctggaacttatgaggaaagagttggaggacgccttgactcaggaggccaacgtggggaaaaagactgtcatttggaaggagaaagtggaaatgcagaggcagcgcttcagattggagtttgagaagcatcgtggctttctggcccaggaggagcaacggcagctgaggcggctggaggcggaggagcgagcgacgctgcagagactgcgggagagcaagagccggctggtccagcagagcaaggccctgaaggagctggcggatgagctgcaggagaggtgccagcgcccggccctgggtctgctggagggtgtgagaggagtcctgagcagaagtaaggctgtcacaaggctggaagcagagaacatccccatggaactgaagacagcatgctgcatccctgggaggagggagctcttaaggaagttccaagtggatgtaaagctggatcccgccacggcgcacccgagtctgctcttgaccgccgacctgcgcagtgtgcaggatggagaaccatggagggatgtccccaacaaccctgagcgatttgacacatggccctgcatcctgggtttgcagagcttctcatcagggaggcattactgggaggttctggtgggagaaggagcagagtggggtttaggggtctgtcaagacacactgccaagaaagggggaaaccacgccatctcctgagaatggggtctgggccctgtggctgctgaaagggaatgagtacatggtccttgcctccccatcagtgcctcttctccaactggaaagtcctcgctgcattgggattttcttggactatgaagccggtgaaatttcattctacaatgtcacagatggatcttatatctacacattcaaccaactcttctctggtcttcttcggccttactttttcatctgtgatgcaactcctcttatcttgccacccacaacaatagcagggtcaggaaattgggcatccagggatcatttagatcctgcttctgatgtaagagatgatcatctctaaaattctgttcccaagatgcagtcctagcgtagcgaacgttcctggagtggggtgaaggatatcaatatactaagttttaacagataccccatttaggtcagcacttgattcgttgttgctgtgaaatatgtccatgggacaaaagagggaatatgaaatatttgcatatgggaagattatagagcataataattttgtaaatggagcaatctcaacctctatttctagatcacattttcttgatgtcttccttcaaattaatgaccttggattacataaggatttctatgcattcattataatttgttattcctttcaatatccttgtatttcaaatcttccatataagaattagacatggcaattcttaaattgattcagaatggtctgatactattccagtatcacctccttaattctgtttctcctcgttttcctgattttccttctcattctctccttccccgctctgtctctctctccctgtcactctctctctcttgttccttattttttgtttcttacctcttactgtttaacctgttgcttccttctggattaatacatttagagccattcctttatatggtcacatttcctatgactttactcaattacttttaaaatcctttctattctgagactaatttttaagaattacaaagctcattcttctgaatctaatatcactaactcctagactttttccgttttctttggatacactttaagtaggaatttatcagaattttcattcaactcgttctttaatgcagatatttactagttataagaccttaaggctgggtgcagtggctcacgcctgtaatcccagcactttgggaggctgaggcgggtggatcacaagctcaggagttcaagaccagcctggccaacatggtgaaaccctgtctctactaaaaaaaaaaaaaaaatagaaaaattagctgggcatggtggcaggagcctgtaatcccagctattctggaggtggagacaggagaattgcttgaaccctggaggcggaggttgcagtgagccaatatctcaccactgtactccagcccagtgcgagactccatctcaaaaaagaaaaaagacctcaaacaacacttctctctctcttttagctgcttgttatggttcctatacatggaacaattatactggcctcactgtgttatggtaaatatttaaggtcatatttgatattgctggtttgaattcagcttttccatttaaatacattataatgatgatgatgaaatcatgataatatttaacttatttttaaagtatattctgtacctttccaacaaaaaggttaaaagtcattgaaggctaaccttactgccttctttgtatcactgtcttctaaataattattatgtctgggtacagtggctcacgcctgtaatcccagcactttgggaggccgaggtgggcagatcacgaggtcaggagattgagaccatcctggctaacacagtgaaaccccgtctctactaaaaatacaaaaagaaattagctgggcgtggtggtgggtgcctgttgtcccagctacttgggaggctgaggcaggagaatggcatgaacccaggaggcagagcttgtagtgagccgagatcgcgccactgcactccagccggggcaacagagcaagactccatctcaaaaataaataaataaataaataaataaataaataaataaataaatattacacaaatgctaaaatgtttaaatggtaaatgcttcaatgctaaccaaatattaattaatggcaaattatttaacattatctgataataatctgcagaaggtttaattttcctcctcaatttgaagttcaagatgtttttctcttccagggagattttttcgactgacatctttaacttaccttccaatcatattactaacgtagccttcttcctagattttttaattgtttgatcatgagcgaacacttctactctctgtgatagatttgcaaacagaggaaataacgcatcctcgtgtccctcttcttggtgttccacaggccatgtgtgccctagccctcgttcatgcaaggtctgtgtagggaaggtggacttcagctcagcaacagcatcccttcccacagggatcaggtgggtggcttgagataccccttccatggggcaccacccattcagtgagacggggaagccctgggtgggagggagaacacctccacatgtcttctactctctccataggatggaatgagtgtcccagtcccaggagtatccatttcccactgtgtagcccagtactctggtctcactgtctctgctgaatcctgtctcactgtgcatattattgtggtttatatcagtcagtaaaccaatgtgagtcttcatctcttgcattcttaggttcatagttttgtgtgtctcctgtaatgactcttctctttccctttccaactcctgaaagattgccactatttcctctggaactttgtttcgttaccagcaaaatcctcgacatccatacccgtttcctggctttccctctcctttcctctgaatggtagtcttttatattcagctgtccacttgacatcaaaatagacattttgaactcaatttgcctaaaacttacccacaaatttctccccaagtctctccctaactgcaacaacaaaaaccacaggcttctccctgtcactggatggcaactccattcttttgattgcttaagccaggcatccgattgagtactttcttgatttctccagcccacatccagtccatcggcaagccctgttggtcctaccttcagaatatgtccggggttcagttgtcctggccaccctgctgctgtaaccatggtcagaactccatcctgcccctctggattatgactttcgtttcctcacagtggtcctgcttgggctctaggcccttccactcccattctctctacagcagctgggctgattcctttagcacccaaggatatgttggcatcacagtgacttagataccatcacaaagacctcccattcaacttagagtgaaagtcagaatcctcacagtgaatccccaggccctagaggatgtgaacccccaggccctagaggatctgaacccccatccctcctctgattatctctcccacccccacttccctttgcattctgctccagctgccctggcctcatggctgggtttccaccaaagcaggcacttcccatcacagggccatttccccgcctgtggcttctgcttgacattcccttttccctgatatccccttgactcattattccctttcttccttaactcttctgagatccagcttctcagtgataccacacagccctactccccccagagcccatctagagctcacctttccagtcgcccttgccaggctcagtggaggctctttgttccccatacagtacgtgtcgtcgtactatattgttaggcttatttaatttatgtatgttttgcctttttgtgctaaatgtaaacaccacaaggggaggtatctttgtctgttgacaatgatacattcaatgtttctcaagcacccccaatgctggtttgtatgtggttatcattcaatctgtatttgttgaatgaataaatgattgactatgtggagagcaaaa

SEQ ID NO: 5 - protein, 486 aa, Homo sapiens
MAWAPPGERLREDARCPVCLDFLQEPVSVDCGHSFCLRCISEFCEKSDGAQGGVYACPQCRGPFRPSGFRPNRQLAGLVESVRRLGLGAGPGARRCARHGEDLSRFCEEDEAALCWVCDAGPEHRTHRTAPLQEAAGSYQVKLQMALELMRKELEDALTQEANVGKKTVIWKEKVEMQRQRFRLEFEKHRGFLAQEEQRQLRRLEAEERATLQRLRESKSRLVQQSKALKELADELQERCQRPALGLLEGVRGVLSRSKAVTRLEAENIPMELKTACCIPGRRELLRKFQVDVKLDPATAHPSLLLTADLRSVQDGEPWRDVPNNPERFDTWPCILGLQSFSSGRHYWEVLVGEGAEWGLGVCQDTLPRKGETTPSPENGVWALWLLKGNEYMVLASPSVPLLQLESPRCIGIFLDYEAGEISFYNVTDGSYIYTFNQLFSGLLRPYFFICDATPLILPPTTIAGSGNWASRDHLDPASDVRDDHL

SEQ ID NO: 6 - RNA, 6319 nt, Homo sapiens
  source 1..6319: mol_type = mRNA
actgccgcctcccggcccctcggagggagccagcccagccgcagccgccgccaccgccgccgccggggccgggccccctcgccgctgccccgggaaggaggtctcggcgcggaagatggccggcggcgtggacggccccatcgggatcccgttccccgaccacagcagcgacatcctgagtgggctgaacgagcagcggacgcagggcctgctgtgcgacgtggtgatcctggtggagggccgcgagttccccacgcaccgctcggtgctggccgcctgcagccagtacttcaagaagctgttcacgtcgggcgccgtggtggaccagcagaacgtgtacgagatcgacttcgtcagcgccgaggcgctcaccgcgctcatggacttcgcctacacggccacgctcaccgtcagcacagccaacgtgggtgacatcctcagcgccgcccgcctgctggagatccccgccgtgagccacgtgtgcgccgacctcctggaccggcagatcctggcggccgacgcgggcgccgacgccgggcagctggaccttgtagatcaaattgatcagcgcaacctcctccgcgccaaggagtacctcgagttcttccagagcaaccccatgaacagcctgccccccgcggccgccgccgccgctgccagcttcccgtggtccgcctttggggcgtccgatgatgacctggatgccaccaaggaggccgtggccgccgctgtggccgccgtggccgcgggcgactgcaacggcttagacttctatgggccgggccccccggccgagcggcccccgacgggggacggggacgagggcgacagcaacccgggtctgtggccagagcgggatgaggacgcccccaccgggggtctctttccgccgccggtggccccgccggccgccacgcagaacggccactacggccgcggcggagaggaggaggccgcctcgctgtcggaggcggcccccgagccgggcgactctccgggcttcctgtcgggagcggccgagggcgaggacggggacgggcccgacgtggacgggctggcggccagcacgctgctgcagcagatgatgtcatcggtgggccgggcgggggccgcggcgggggacagcgacgaggagtcgcgggccgacgacaagggcgtcatggactactacctgaagtacttcagcggcgcccacgacggcgacgtctacccggcctggtcgcagaaggtggagaagaagatccgagccaaggccttccagaagtgccccatctgcgagaaggtcatccagggcgccggcaagctgccgcgacacatccgcacccacacgggcgagaagccctacgagtgcaacatctgcaaggtccgcttcaccaggcaggacaagctgaaggtgcacatgcggaagcacacgggcgagaagccgtacctgtgccagcagtgcggcgccgcctttgcccacaactacgacctgaagaaccacatgcgcgtgcacacgggcctgcgcccctaccagtgcgacagctgctgcaagaccttcgtccgctccgaccacctgcacagacacctcaagaaagacggctgcaacggcgtcccctcgcgccgcggccgcaagccccgcgtccggggcggggcgcccgaccccagcccgggggccaccgcgacccccggcgcccccgcccagcccagctcccccgacgcccggcgcaacggccaggagaagcactttaaggacgaggacgaggacgaggacgtggccagccccgacggcttgggccggttgaatgtagcgggcgccggtggaggaggtgacagcggaggtggccccggggccgccaccgacggtaacttcacagccggactcgcctaaaaaccaaaaagagaaaacagaaacccgagaaagagagagacagagagagagaaaaaaaatcacccaccacccccccaaaaacacaaaaaaagaaaatctatctatatacagatatctatatctatatatatatatacagatatatatatatgacgcgtcacagaatctagggtagcgctttctcagatttccctcctttctgacgtttttctccctccgcaggggccccggccctccctggctccccttccccccaccaccccatcgctgggtttcggggcttggtttggggttttttgtaggacacaaggaatccgagaccccgcacagccccctgggcacccggcatggggcctggggcccgatccgaggccctgggctggggggagggtagacgtgggggcgctggggggggactggggtgggcttttaatttcctcccctcgctggtttctatgagtctttcagacaagaccttaaatgatttctgtctgctctgagcggacgttaaaatgggcccccgtcccccgacccgcaccctccttcctcagggcacttactaagggaggggtctccctctccatctccccagtggcctccccgcctccaaccctgcctgcggcctccccccgtcgcccaccccacgtctcctggccactgagacacaaacctatttatttctaggcctggagaaaggagatcggactggggttcccggtggggcgccaggatggctcctgggggtgctcctgccgccttccttcacggcacttacaaccggcgggacccccagggaccacccctcagggcgcccccccacccccgcccggtccacctagacccccacgtttggagattcaaaacttctgtcttcgtcctctcccccgagccccctctcccaaatttttaaagcactttttagatttttttttctctttcctccttaaaaacaaaatttatatatagatatatatatatatataaataatatacttttcctcagaggagcaggcaacagtgtgggataaacagagtcacgatcagaggaaccccagggtctggtgatggcagggatggggggagagagagaaaatccacaaattccaatgtcacaaaagcaataaaacaaactagaaaaaaaaaaggttttacaaaatgaaaggaaggaaaaaaaaaaaggcaaccaaccacattagaagtcttggcactttgtaacggaacgggtactacactttatcttaattcttaatttaaaaacatgtttacaagttacaaccaacttctatgaaaagttgaaaagacaaaaaaaaaaaaaaaaagcgagcgagagagagagcgagagagagagcgagagcagaagaaattcctaaaagtcgatttatttttgtacaaaataataaaaaaaaaaacccaccacaaacgtagaatccacttctgttccccaaaaagcgagaaggggggttcaggaggaagccatcgcaggggacctgggagacgccccgaggtgtttgtgcttcacccccagacgtcagcctcgaaggcaggactgtggggtgttcgtgctgtgttccccccgctccccctttctgtccccttttttggttctgacgtgaagaggtcttagcgcccgcttctgtccacggggtctctccttcctcctccctagctcagggatgggccttccagccggagcaccccgatccccatccggcaccccccaatcccccaacacgcctgtccctcccgcatggccaccaaggagctggaccttggatgcgcctaccctgctgaggtgggtgacaggggccccccacctccagggccttagaaccaccgcccctctccccaccccaggcacccctctttttactcaaaggcactgactgtaatccagggggactgggacctgcctccccccaacctctggctcccacaaggcccggtgttgaccgagccacaggccacggacaggggccggggttggggagactatgtcgccagatgccaggacgccctcaccccgtttgcatatgcaatgctagcatgggaccccgaaaatagacgctctgctgcactgagacttcttgtcaatgcccaaccggcgggggggtgtctccctgcccccgacccccccatacccccttctctgtgacacacacatcttctcgtctctttttctttcattgttaaagggaagctttttaagaaggcaattttcatattgtttctacaggatggttttggttcccttcccttcccacccccccttaagcctgtcagccccctccaaatgtctcaggatcccccctctcccctggggctgggtgacagcaccccggctgcgttcacaccccagtgtcacagggcgagctgttctggagagaaaaccatctgtcgtggctgagcggggagcttgaacacccaggccagggacacccctccccagctcccagagaggccccctgaggggtgagccctctttccaccttcccctatccatgcaccccctcgcaataaaaccaactctaaaatcacagctgtcgtcctagccagtgggggcgaccggacttggggggtggagccctctgggacttccgtaggaacaagggctgcggcccaccgcgacacttacacagacctcggggattgcactaaaccctcgttcctagctccgcactcagcttcgcctgtcctgcccgcccactttgccttaactacccgcccgtcctgggggccacagcctctgcatgggcccagagccgggacccccccagcccagccccgccctccccagactccgcgcaatcacatactgtatatagacgtgaatcgattttatttttattctttaaattaaggtcgtgataaagtgttgccaaagatacctgctgaattctcgcgtttcaggaaacaaacaaacaaaaaaaaatgatatttgaggagggtcgtgttgactccatatgaaaggacacagctcaaagcttttttgtttggttgtttggggttttttgtgttttctttttttggggtgttttttttttaactgcctggtacaaaaaaaaaaagagaaaaaaaaaaaagaaaaacaatgcgaaattgttatttccattctcatggtgaagttgcgtggacgcgtgtgtgcgtgtgtgcaagagagcgggagtgaggtccaggctggggttggggggcttcaggcgggggcgcccgggggccggggaggtggccgggccggagcccccgtctgcagtgccccccagcctgccgggcccaggagagagagagaagcatctttgctactagctgttgctgctacctgcctctgccccccgacgccccccgccttttgagattaaggaaaaaaaaaaaaagtcaaaaaagtttttaaaaatgaaaaaaaaaaattataaaccagtgaatgtaaaatgccggagcaggcccggcctggcatgggtgtggacctgcagccaggcaggctcgagcgggcgataccaaagtctgcccccccaccattgtggccatgcagtcctgtcactgtctttttgcttccttccgaggggggtcccccagcctcttccagggtcttcccctggaagtgggcggctgcagggaaggtgggggacaggggtctttgcacgattcagaccccggggccgtggcaggagcggtcacctcacaggtggtgacactgaggcaggggcctcggggtgccccctcccgcccggcaaccagaatggttggaggcaagacagagagaatgaaaggaaaaacagaagaaaaaaaaatattaaaaaccaacaaaaaaagcaaaaatcctattttttgagaaagaaagatatttatatttgcagttttattttaaaaagttatttaagttgaagcagccttcctggaggtgggggggggggggtggtgggtggctggcgcaggacgggtcaggggcctggaggctgggggtgccccaggagctacaacctcagagttaagactagctcgcattaaatacatagatttacggggggggggggggggggccgggcccagggggtggagggggccagggagacccccatccctcgccggggctgcctggaggctgtggaccaggatccgatgcccaggtcccgccccccaccccaccccaggcccagaatcgaggtgccttggactttggaggggccaggcctggtgaatggggggcggggcggcgccctcagggtacagagcacagacagatagacattccagagactgtattgagagtctttataaagtgtgggagatttaaaaaaaaaaaaaactgataaaaatgcactttttgggagtggggagggagaagctttaaaagtaataaaaaacaaacaaaaacacaaaagatgaaaaaacaaaaaaattcatttttcttgtacataaaaaaaaaaaaagaaccactaaacgcagcctgttacgacc

SEQ ID NO: 7 - protein, 584 aa, Homo sapiens
MAGGVDGPIGIPFPDHSSDILSGLNEQRTQGLLCDVVILVEGREFPTHRSVLAACSQYFKKLFTSGAVVDQQNVYEIDFVSAEALTALMDFAYTATLTVSTANVGDILSAARLLEIPAVSHVCADLLDRQILAADAGADAGQLDLVDQIDQRNLLRAKEYLEFFQSNPMNSLPPAAAAAAASFPWSAFGASDDDLDATKEAVAAAVAAVAAGDCNGLDFYGPGPPAERPPTGDGDEGDSNPGLWPERDEDAPTGGLFPPPVAPPAATQNGHYGRGGEEEAASLSEAAPEPGDSPGFLSGAAEGEDGDGPDVDGLAASTLLQQMMSSVGRAGAAAGDSDEESRADDKGVMDYYLKYFSGAHDGDVYPAWSQKVEKKIRAKAFQKCPICEKVIQGAGKLPRHIRTHTGEKPYECNICKVRFTRQDKLKVHMRKHTGEKPYLCQQCGAAFAHNYDLKNHMRVHTGLRPYQCDSCCKTFVRSDHLHRHLKKDGCNGVPSRRGRKPRVRGGAPDPSPGATATPGAPAQPSSPDARRNGQEKHFKDEDEDEDVASPDGLGRLNVAGAGGGGDSGGGPGAATDGNFTAGLA

SEQ ID NO: 8 - RNA, 2792 nt, Homo sapiens
  source 1..2792: mol_type = mRNA
gtgtgtggacggaattcgggaccgactgacggccggccggcttcccggaactggaaggttacattgattacccacctagtacaacatcttacgggaagagcatagtatttcctagaggaatatgaacataacaggaaggtatcattggctctgaattaaatttgaacttgtcccctgaatagctacaggttttggaagctgaatcaatgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccagaacctgttccatggagagaagaggatggaaagtcaggctgcagtgacttgaatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgctgagccactaatgggcagtagtttgcttgaagaggcttcagtacccagtaaaaacctggtgtctatgaattcccagcccagccttggtggagagtccttgaacctaccaaataccaattctatcctgggagttgatgatgaggtgcttgctgaaggatccccacgttccctgtcttcagtgcctgatgtgacacatcacctggtgaccatgcagtcagggaggcaatcatatgaagtttctgtcttaactgcagtaaatccacaagagagtctcgctccgttgcccaggctggagtgcagtggtgcgttctcggctcactgcaacctctgcctcccaggttcaagcgattctcctgcctcagcctcctgagtagctgctactacagttactaaaccaaggagatttaactgaaagacggacatgagcgtgggtgctgactcctggaagagcaactctatctgatctcaaaatgcgtatactgggaacaggatgccttagcccacaacagaaccagaatgaatctttgaaggcacaagactctgcttttgccactcttcctctttcctggtatagaagatggatgtaggagagcttcttttctaactaccatctgatcagacaaggaatgaagcaatgactgtgggctgggaaactgtacctacctctcttcccactgcaaatttctgggatagaccaaaagtgaatttgattatgtgttggctgaagttcttcattctgactgttgaggggaggttttcctttgaagagttttcatcccagactcagctgtcttttcacatggatgaaataattcctgctaccaacaacagagcttcaccaggaagttgagttttcaagatgccttgttgctttgaagaagggagtgatgtcaattctcttgttacattctccctttagcaacctgagtaagagactctctgccactgggctgcaaaaaaataaattacttgaatctccccttggcccaggctgaggtactatcttgtcctatacacttctacttggtcactttgctttcttcgttaatggaacatacagtagcatgttaagagggatttcatgtttttgtttttttaaagttaaaaattacatgatgcagagattcaggttttcctttaaataaacaaaacagcccagtcagttctttggctcttgttttatacaaaatttgttttcttagagattaagaatttaatctttccctttaaaatagtgtattgatttacccttaggattccataccagtaaaactgaaccgaggagtcttaggaaacaacaagaacatcttcatttcttaagcccaggtgatagttactctgtcaccaccaaaaaagacgcatctgagaaaatggcaataaaaacagatacttctgaatttttcca

SEQ ID NO: 9 - protein, 516 aa, Homo sapiens
MLSDELESKPELLVQFVQNTSIPLGQGLVESEAKDITCLSLLPVTEASECSRLMLPEPVPWREEDGKSGCSDLNDTTNHSNSSKEVPSSAVLRSLRVNVGPDGEETRAQTVQKSPEFLSTSESSSLLQDLQPSDSTSFILLNLTRAGLGSSAEHLVFVQDEAEDSGNDFLSSESTDSSIPWFLRVQELAHDSLIAATRAQLAKNAKTSSNGENVHLGSGDGQSKDSGPLPQVEKKLKCTVEGCDRTFVWPAHFKYHLKTHRNDRSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGAAGSQEQEQTAEPLMGSSLLEEASVPSKNLVSMNSQPSLGGESLNLPNTNSILGVDDEVLAEGSPRSLSSVPDVTHHLVTMQSGRQSYEVSVLTAVNPQESLAPLPRLECSGAFSAHCNLCLPGSSDSPASAS

SEQ ID NO: 10 - RNA, 6102 nt, Homo sapiens
  source 1..6102: mol_type = mRNA
gtctctgtccatccagactcctgacgttcaagttcgcagggacgtcacgtccgcacttgaacttgcagctcaggggggcttttgccatttttttcatctctctctctctctctccctctatctctcttctctctctctccctctttttttttttttttttttttttttttttgcttaaaaaaaagccatgacggctctcccacaattcatcttccctgcgccatctttgtattatttctaatttattttggatgtcaaaaggcactgatgaagatattttctctggagtctccttctttctaacccggctctcccgatgtgaaccgagccgtcgtccgcccgccgccgccgccgccgccgccgccgcccgccccgcagcccaccatgtctcgccgcaagcaaggcaaaccccagcacttaagcaaacgggaattctcgcccgagcctcttgaagccattcttacagatgatgaaccagaccacggcccgttgggagctccagaaggggatcatgacctcctcacctgtgggcagtgccagatgaacttcccattgggggacattcttatttttatcgagcacaaacggaaacaatgcaatggcagcctctgcttagaaaaagctgtggataagccaccttccccttcaccaatcgagatgaaaaaagcatccaatcccgtggaggttggcatccaggtcacgccagaggatgacgattgtttatcaacgtcatctagaggaatttgccccaaacaggaacacatagcagataaacttctgcactggaggggcctctcctcccctcgttctgcacatggagctctaatccccacgcctgggatgagtgcagaatatgccccgcagggtatttgtaaagatgagcccagcagctacacatgtacaacttgcaaacagccattcaccagtgcatggtttctcttgcaacacgcacagaacactcatggattaagaatctacttagaaagcgaacacggaagtcccctgaccccgcgggttggtatcccttcaggactaggtgcagaatgtccttcccagccacctctccatgggattcatattgcagacaataacccctttaacctgctaagaataccaggatcagtatcgagagaggcttccggcctggcagaagggcgctttccacccactccccccctgtttagtccaccaccgagacatcacttggacccccaccgcatagagcgcctgggggcggaagagatggccctggccacccatcacccgagtgcctttgacagggtgctgcggttgaatccaatggctatggagcctcccgccatggatttctctaggagacttagagagctggcagggaacacgtctagcccaccgctgtccccaggccggcccagccctatgcaaaggttactgcaaccattccagccaggtagcaagccgcccttcctggcgacgccccccctccctcctctgcaatccgcccctcctccctcccagcccccggtcaagtccaagtcatgcgagttctgcggcaagacgttcaaatttcagagcaacctggtggtgcaccggcgcagccacacgggcgagaagccctacaagtgcaacctgtgcgaccacgcgtgcacccaggccagcaagctgaagcgccacatgaagacgcacatgcacaaatcgtcccccatgacggtcaagtccgacgacggtctctccaccgccagctccccggaacccggcaccagcgacttggtgggcagcgccagcagcgcgctcaagtccgtggtggccaagttcaagagcgagaacgaccccaacctgatcccggagaacggggacgaggaggaagaggaggacgacgaggaagaggaagaagaggaggaagaggaggaggaggagctgacggagagcgagagggtggactacggcttcgggctgagcctggaggcggcgcgccaccacgagaacagctcgcggggcgcggtcgtgggcgtgggcgacgagagccgcgccctgcccgacgtcatgcagggcatggtgctcagctccatgcagcacttcagcgaggccttccaccaggtcctgggcgagaagcataagcgcggccacctggccgaggccgagggccacagggacacttgcgacgaagactcggtggccggcgagtcggaccgcatagacgatggcactgttaatggccgcggctgctccccgggcgagtcggcctcggggggcctgtccaaaaagctgctgctgggcagccccagctcgctgagccccttctctaagcgcatcaagctcgagaaggagttcgacctgcccccggccgcgatgcccaacacggagaacgtgtactcgcagtggctcgccggctacgcggcctccaggcagctcaaagatcccttccttagcttcggagactccagacaatcgccttttgcctcctcgtcggagcactcctcggagaacgggagtttgcgcttctccacaccgcccggggagctggacggagggatctcggggcgcagcggcacgggaagtggagggagcacgccccatattagtggtccgggcccgggcaggcccagctcaaaagagggcagacgcagcgacacttgtgagtactgtgggaaagtcttcaagaactgtagcaatctcactgtccacaggagaagccacacgggcgaaaggccttataaatgcgagctgtgcaactatgcctgtgcccagagtagcaagctcaccaggcacatgaaaacgcatggccaggtggggaaggacgtttacaaatgtgaaatttgtaagatgccttttagcgtgtacagtaccctggagaaacacatgaaaaaatggcacagtgatcgagtgttgaataatgatataaaaactgaatagaggtatattaatacccctccctcactcccacctgacaccccctttttcaccactccccttccccatcgccctccagccccactccctgtaggatttttttctagtcccatgtgatttaaacaaacaaacaaacaaacagaagtaacgaagctaagaatatgagagtgcttgtcaccagcacacctgttttttttctttttctttttcttttttctttttccttttttttttttttcctttatgttctcaccgtttgaatgcatgatctgtatggggcaatactattgcattttacgcaaactttgagcctttctcttgtgcaataatttacatgttgtgtatgtttttttttaaacttagacagcatgtatggtatgttatggctattttaaattgtccctaattcgttgctgagcaaacatgttgctgtttccagttccgttctgagagaaaaagagagagagagagaaaaagaccatgctgcatacattctgtaatacatatcatgtacagttttattttataacgtgaggaggaaaaacagtctttggattaaccctctatagacagaatagatagcactgaaaaaaaatctctatgagctaaatgtctgtctctaaagggttaaatgtatcaattggaaaggaagaaaaaaggccttgaattgacaaattaacagaaaaacagaacaagtttattctatcatttggttttaaaatatgagtgccttggatctattaaaaccacatcgatggttctttctacttgttataaacttgtagcttaattcagcattgggtgaggtaataaaccttaggaactagcatataattctatattgtatttctcacaacaatggctacctaaaaagatgacccattatgtcctagttaatcatcatttttcctttagtttaattttataaacaaaactgattataccagtataaaagctactttgctcctggtgagagcttaaaagaaatgggctgttttgcccaaagttttattttttttaaacaatgattaaattgaatgtgtaatgtgcaaaagccctggaacgcaattaaatacactagtaaggagttcattttatgaagatatttgctttaataatgtctttttaaaaatactggcaccaaaagaaatagatccagatctacttggttgtcaagtggacaatcaaatgataaactttaagaccttgtataccatattgaaaggaagaggctgacaataaggtttgacagaggggaacagaagaaaataatatgatttattagcacaacgtggtactatttgccatttaaaactagaacaggtatataagctaatattgatacaatgatgattaactatgaattcttaagacttgcatttaaatgtgacattcttaaaaaaagaagagaaagaattttaagagtagcagtatatatgtctgtgctccctaaaagttgtacttcatttcttttccatacactgtgtgctatttgtgttaacatggaagaggattcattgtttttatttttatttttttaattttttcttttttattaagctagcatctgccccagttggtgttcaaatagcacttgactctgcctgtgatatctgtatcttttctctaatcagagatacagaggttgagtataaaataaacctgctcagataggacaattaagtgcactgtacaattttcccagtttacaggtctatacttaagggaaaagttgcaagaatgctgaaaaaaaattgaacacaatctcattgaggagcattttttaaaaactaaaaaaaaaaaaactttgccagccatttacttgactattgagcttacttacttggacgcaacattgcaagcgctgtgaatggaaacagaatacacttaacatagaaatgaatgattgctttcgcttctacagtgcaaggatttttttgtacaaaacttttttaaatataaatgttaagaaaaattttttttaaaaaacacttcattatgtttaggggggaactgcattttagggttccattgtcttggtggtgttacaagacttgttatccatttaaaaatggtagtggaaattctatgccttggatacacaccgctcttcaggttgtaaaaaaaaaaaacatacattggggaaaggtttaagattatatagtacttaaatataggaaaatgcacactcatgttgattcctatgctaaaatacatttatggtcttttttctgtatttctagaatggtatttgaattaaatgttcatctagtgttaggcactatagtatttatattgaagcttgtatttttaactgttgcttgttctcttaaaaggtatcaatgtaccttttttggtagtggaaaaaaaaaagacaggctgccacagtatatttttttaatttggcaggataatatagtgcaaattatttgtatgcttcaaaaaaaaaaaaaagagagaaacaaaaaagtgtgacattacagatgagaagccatataatggcggtttgggggagcctgctagaatgtcacatggatggctgtcataggggttgtacatatccttttttgttcctttttcctgctgccatactgtatgcagtactgcaagctaataacgttggtttgttatgtagtgtgctttttgtccctttccttctatcaccctacattccagcatcttaccttcatatgcagtaaaagaaagaaagaaaaaaaaaggaaaaaaaaaaaaaaaccaatgttttgcagtttttttcattgccaaaaactaaatggtgctttatatttagattggaaagaatttcatatgcaaagcatattaaagagaaagcccgctttagtcaatacttttttgtaaatggcaatgcagaatattttgttattggccttttctattcctgtaatgaaagctgtttgtcgtaacttgaaattttatcttttactatgggagtcactatttattattgcttatgtgccctgttcaaaacagaggcacttaatttgatcttttatttttctttgtttttatttttttttttatttagatgaccaaaggtcattacaacctggctttttattgtatttgtttctggtctttgttaagttctattggaaaaaccactgtctgtgtttttttggcagttgtctgcattaacctgttcatacacccattttgtccctttattgaaaaaataaaaaaaattaaagtaca

SEQ ID NO: 11 - protein, 835 aa, Homo sapiens
MSRRKQGKPQHLSKREFSPEPLEAILTDDEPDHGPLGAPEGDHDLLTCGQCQMNFPLGDILIFIEHKRKQCNGSLCLEKAVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDESRALPDVMQGMVLSSMQHFSEAFHQVLGEKHKRGHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLSFGDSRQSPFASSSEHSSENGSLRFSTPPGELDGGISGRSGTGSGGSTPHISGPGPGRPSSKEGRRSDTCEYCGKVFKNCSNLTVHRRSHTGERPYKCELCNYACAQSSKLTRHMKTHGQVGKDVYKCEICKMPFSVYSTLEKHMKKWHSDRVLNNDIKTE

SEQ ID NO: 12 - protein, 120 aa, synthetic construct
QVQLVESGGGLVQAGGSLRLSCAASGSIFSFYAMGWYRQAPGKEREFVASITWRDDSTYYADSVKGRFTISRDNAKNTVYLQMNSLKPEDTAVYYCAADTDDYTVEMDYWGQGTQVTVSS

SEQ ID NO: 13 - protein, 119 aa, synthetic construct
QVQLVESGGGLVQAGGSLRLSCAASGSTFSDYAMGWYRQAPGKERELVAVITASDDITYYADSVKGRFTISRDNAKNTVYLQMNSLKPEDTAVYYCAAAVTPGQDLDYWGQGTQVTVSS

SEQ ID NO: 14 - protein, 119 aa, synthetic construct
QVQLVESGGGLVQAGGSLRLSCAASGSTFSGYAMGWYRQAPGKERELVAAITSSGASTYYADSVKGRFTISRDNAKNTVYLQMNSLKPEDTAVYYCAALDEGYLDYDSWGQGTQVTVSS

SEQ ID NO: 15 - protein, 117 aa, synthetic construct
QVQLVESGGGLVQAGGSLRLSCAASGFIFDSYAMGWYRQAPGKERELVAAITSSGSSTYYADSVKGRFTISRDNAKNTVYLQMNSLKPEDTAVYYCAALDYVIDGYWGQGTQVTVSS

SEQ ID NO: 16 - protein, 117 aa, synthetic construct
RVQLVESGGGLVQAGGSLRLSCAADGFDFKSYAMGWYRQAPGREDELVAAITASGDYTYYADSVKGRFTISRDNAKNTVYLQMNSLKPDDTAVYYCAALDYVAEGYWGQGTQVTVSS

SEQ ID NO: 17 - protein, 119 aa, synthetic construct
QVQLVKSGGGLVQAGDSLRLSCAASGSTFSGYAMGWYRQAPGKERELVAAITSSGASTYYADSVRGRFTISRDNAKNTVYLQMNSLKPEDTAVYYCAALDEGYLDYDSWGQGTQVTVSS

SEQ ID NO: 18 - protein, 117 aa, synthetic construct
RVQLVESGGGLVQAGGSLRLSCAASGFIFDSYAMGWYRQAPGKEMELVAAITSSGSSTYYADSVKGRFTISRDNAKNTVYLQMNSLKPEDTAVYYCAALDYVIDGYWGQGTQVTVSS

SEQ ID NO: 19 - protein, 117 aa, synthetic construct
RVQLVESGGGLVQAGGSLRLSCAASGFIFDSYAMGWYRQAPGKEDELVAAITSSGSSTYYADSVKGRFTISRDNAKNTVYLQMNSLKPEDTAVYYCAALDYVIDGYWGQGTQVTVSS

SEQ ID NO: 20 - protein, 120 aa, synthetic construct
QVQLVESGGGLVQAGGSLHLSCAVSGSIFSFYAMGWYRQAPGKEREFVASITRRDDSTYYADSVEGRFAISRDNAKNTVYLQMNSLKPEDTAVYYCAADTDDYTVEMDYWGQGTQVTVSS

SEQ ID NO: 21 - protein, 120 aa, synthetic construct
QVQLVESGGGLVQAEGSLRLSCAASGSIFSFYAMGWYRQAPGKEREFVASITWRDDSTYYADSVTGRFTISRDNAKNTVYLQMNSLKPEDTAVYYCAADTDDYSVEMDYWGQGTQVTVSS

SEQ ID NO: 22 - protein, 120 aa, synthetic construct
QVQLVESGGGLVQAGGCLRLSCAASGSIFSFYAMGWYRQAPGNEREFVASITWRDDSTYYADSVKGRFTISRDNAKNTVYLQMNSLKPEDTAVYYCAADTDDYTVEMDYWGQGTHVTVSS

SEQ ID NO: 23 - protein, 120 aa, synthetic construct
QVQLVESGGDLVQAGASLRLSCAASGSIFSFYAMGWYRQAPGKEREFVASITWRDDSTYYADSVKGRFTISRDNARNTVYLQMNNLKPEDTAVYYCAADTDDYTVEMDYWGQGTQVTVSS

SEQ ID NO: 24 - protein, 120 aa, synthetic construct
QVQLVESGGGLVQAGGSLRLSCAASGSIFSFYAMGWYRQAPGKEREFVAFITWRDDSTYYADSVKGRFRISRDNAKNTVYLQMNSLKPEDTAVYYCAADTDDYTVEMDYWGQGTQVTVSS

SEQ ID NO: 25 - protein, 120 aa, synthetic construct
QVQLVESGGDLVQAGGSLRLSCAASGSIFSFYAMGWYRQAPGKEREFVASITWRDDSTYYADSVKGRFTISRDNAKNTVYLQMNRLKPEDTAVYYCAADTDDYTVEMDYWGQGTQVTVSS

SEQ ID NO: 26 - protein, 120 aa, synthetic construct
QVQLVESGGGLVQAGGSLRLSCAASGSIFSFYAMGWYRQAPGKEREFVASITWRDDSTYYADSVMGRFTISRDNAKNTVYLQMNSLKPEDTAVYYCAADTDDYTVEMDYWGQGTQVTVSS

SEQ ID NO: 27 - protein, 119 aa, synthetic construct; identical to SEQ ID NO: 17

SEQ ID NO: 28 - protein, 117 aa, synthetic construct
QVQFVESGGGFVQAGGSLRLSCAASGFIFDSYAMGWYSQAPGKERELVAAITSSGSSTYYADSVKGRFTISRDNAKNTVYLQMNSLKPEDTAVYYCAALDYVIDGYWGQGTQVTVSS

SEQ ID NO: 29 - protein, 117 aa, synthetic construct
QVQLVESGGGLVQAGGSLRLSCAASGFIFDSYSMGWYRQAPGKEGELVAAITSSGSSTYYADSVNGRFTISRDNAKNTVYLQMNSLKPEDTAVYYCAALDYVIDGYWGQGTQVTVSS

SEQ ID NO: 30 - protein, 117 aa, synthetic construct
QVQLVESGGGLVQAGGSLRLSCAASGFIFDSYAMGWYRQAPGKEGELVAAITSSGSSTYYADSVKGRFTISRDNAKNTVYLQMNSLKPEDTAVYYCAALDYVIDGYWGQGTQVSVSS

SEQ ID NO: 31 - protein, 117 aa, synthetic construct
QVQLVESGGGLVQAGGSLRLSCAASGFIFDSYAMGWYRQAPGKEGELVAAITSSGSSTYYADSVKGRFTISRDNAKNTVYLQMNSLKPEDTAVYYCAALDYVIDGYWGQGTQVTVSS

SEQ ID NO: 32 - protein, 117 aa, synthetic construct
RVQLVESGGGLVQAGGSLRLSCAADGFDFSSYAMGWYRQAPGTQDELVAAITASGSYTYYSDSVKGRFTISRDNAKNTVYLQMNSLKPDDTAVYYCAALSYVAEGYWGQGTQVTVSS

SEQ ID NO: 33 - protein, 117 aa, synthetic construct
RVQLVESGGGLVQAGGSLRLSCAADGFDFKSYAMGWYRQAPGYEDELVAAITASGSYTYYSDSVKGRFTISRDNAKNTVYLQMNSLKPEDTAVYYCAALSYVAEGYWGQGTQVTVSS

SEQ ID NO: 34 - protein, 118 aa, synthetic construct
QVQLVESGGGLVQAGGSLRLSCAASGSIFVNNAMGWYRQAPGKERELVAAISASGGSTYYADSVKGRFTISRDNAKNTVYLQMNSLKPEDTAVYYCAADQDVYPYEYWGQGTQVTVSS

SEQ ID NO: 35 - protein, 118 aa, synthetic construct
QVQLVESGGGLVQAGGSLRLSCAASGSIFVNNAMGWYRQAPGKERELVAAISASGGSTYYADSVKGRFTISRDNAKNTVYLQMNSLKPEDTAVYYCAADQDGYPYEYWGQGTQVTVSS

SEQ ID NO: 36 - protein, 118 aa, synthetic construct
QVQLVESGGGLVQAGGSLRLSCAASGSIFVNNAMGWYRQAPGKERELVAAISASGGSTYYADSVKGRFTISRDNAKNTVYLQMNSLKPEDTAVYYCAADQDVYPYEYLGQGTQVTVSS

SEQ ID NO: 37 - protein, 8 aa, synthetic construct
SIFVNNAM

SEQ ID NO: 38 - protein, 15 aa, synthetic construct
ELVAAISASGGSTYY

SEQ ID NO: 39 - protein, 11 aa, synthetic construct
ADQDVYPYEYW

SEQ ID NO: 40 - protein, 11 aa, synthetic construct
ADQDGYPYEYW

SEQ ID NO: 41 - protein, 11 aa, synthetic construct
ADQDVYPYEYL

SEQ ID NO: 42 - protein, 116 aa, synthetic construct
QVQLVESGGGSVQPGGSLTLSCTASGFPLSMWGMTWLRQAPGKGLERVSGIASDSTNTYYADSVKGRFAISRDNAKNKLFLQMNSLKPEDTAVYFCARKWTNWDLKGQGTQVTVSS

SEQ ID NO: 43 - protein, 9 aa, synthetic construct
GSIFSFYAM

SEQ ID NO: 44 - protein, 9 aa, synthetic construct
GSTFSDYAM

SEQ ID NO: 45 - protein, 9 aa, synthetic construct
GSTFSGYAM

SEQ ID NO: 46 - protein, 9 aa, synthetic construct
GFIFDSYAM

SEQ ID NO: 47 - protein, 9 aa, synthetic construct
GFDFKSYAM

SEQ ID NO: 48 - protein, 9 aa, synthetic construct
GFIFDSYSM

SEQ ID NO: 49 - protein, 9 aa, synthetic construct
GFDFSSYAM

SEQ ID NO: 50 - protein, 9 aa, synthetic construct
GFDFKSYAM

SEQ ID NO: 51 - protein, 15 aa, synthetic construct
EFVASITWRDDSTYY

SEQ ID NO: 52 - protein, 15 aa, synthetic construct
ELVAVITASDDITYY

SEQ ID NO: 53 - protein, 15 aa, synthetic construct
ELVAAITSSGASTYY

SEQ ID NO: 54 - protein, 15 aa, synthetic construct
ELVAAITSSGSSTYY

SEQ ID NO: 55 - protein, 15 aa, synthetic construct
ELVAAITASGDYTYY

SEQ ID NO: 56 - protein, 15 aa, synthetic construct
EFVASITRRDDSTYY

SEQ ID NO: 57 - protein, 15 aa, synthetic construct
EFVASITWRDDSTYY

SEQ ID NO: 58 - protein, 15 aa, synthetic construct
EFVAFITWRDDSTYY

SEQ ID NO: 59 - protein, 15 aa, synthetic construct
ELVAAITASGSYTYY

SEQ ID NO: 60 - protein, 13 aa, synthetic construct
ADTDDYTVEMDYW

SEQ ID NO: 61 - protein, 12 aa, synthetic construct
AAVTPGQDLDYW

SEQ ID NO: 62 - protein, 12 aa, synthetic construct
ALDEGYLDYDSW

SEQ ID NO: 63 - protein, 10 aa, synthetic construct
ALDYVIDGYW

SEQ ID NO: 64 - protein, 10 aa, synthetic construct
ALDYVAEGYW

SEQ ID NO: 65 - protein, 13 aa, synthetic construct
ADTDDYSVEMDYW

SEQ ID NO: 66 - DNA, 12 bp, unidentified
  source 1..12: mol_type = unassigned DNA
catcccataata

SEQ ID NO: 67 - protein, 24 aa, unidentified
YTCTTCKQPFTSAWFLLQHAQNTH

SEQ ID NO: 68 - protein, 23 aa, unidentified
KSCEFCGKTFKFQSNLVVHRRSH

SEQ ID NO: 69 - protein, 25 aa, unidentified
YKCNLCDHACTQASKLKRHMKTHMH

SEQ ID NO: 70 - protein, 23 aa, unidentified
DTCEYCGKVFKNCSNLTVHRRSH

SEQ ID NO: 71 - protein, 23 aa, unidentified
YKCELCNYACAQSSKLTRHMKTH

SEQ ID NO: 72 - protein, 24 aa, unidentified
YKCEICKMPFSVYSTLEKHMKKWH